SEQUENCE LISTING (84 sequences)

SEQ ID NO: 1 - protein, 30 aa, synthetic construct
  REGION 1..30: note = (A8S, A30E) GLP-1(7-36)
HSEGTFTSDVSSYLEGQAAKEFIEWLVKGR

SEQ ID NO: 2 - protein, 30 aa, synthetic construct
  REGION 1..30: note = (A8G, G22E, R36G) GLP1(7-36)
HGEGTFTSDVSSYLEEQAAKEFIAWLVKGG

SEQ ID NO: 3 - protein, 39 aa, synthetic construct
  REGION 1..39: note = Exendin 4 (1-39)
HGEGTFTSDLSKQMEEEAVRLFIEWLKNGGPSSGAPPPS

SEQ ID NO: 4 - protein, 28 aa, synthetic construct
  REGION 1..28: note = Exendin 4 (1-28)
HGEGTFTSDLSKQMEEEAVRLFIEWLKN

SEQ ID NO: 5 - protein, 10 aa, synthetic construct
  REGION 1..10: note = First linker peptide 1-1
APAPAPAPAP

SEQ ID NO: 6 - protein, 20 aa, synthetic construct
  REGION 1..20: note = First linker peptide 1-2
APAPAPAPAPAPAPAPAPAP

SEQ ID NO: 7 - protein, 30 aa, synthetic construct
  REGION 1..30: note = First linker peptide 1-3
APAPAPAPAPAPAPAPAPAPAPAPAPAPAP

SEQ ID NO: 8 - protein, 40 aa, synthetic construct
  REGION 1..40: note = First linker peptide 1-4
APAPAPAPAPAPAPAPAPAPAPAPAPAPAPAPAPAPAPAP

SEQ ID NO: 9 - protein, 50 aa, synthetic construct
  REGION 1..50: note = First linker peptide 1-5
APAPAPAPAPAPAPAPAPAPAPAPAPAPAPAPAPAPAPAPAPAPAPAPAP

SEQ ID NO: 10 - protein, 24 aa, synthetic construct
  REGION 1..24: note = First linker peptide 1-6
ASAPAPAPAPAPAPAPAPAPAPGS

SEQ ID NO: 11 - protein, 14 aa, synthetic construct
  REGION 1..14: note = First linker peptide 1-7
ASAPAPAPAPAPGS

SEQ ID NO: 12 - protein, 12 aa, synthetic construct
  REGION 1..12: note = First linker peptide 1-8
ASGGGGSGGGGS

SEQ ID NO: 13 - protein, 42 aa, synthetic construct
  REGION 1..42: note = First linker peptide 1-9
ASGGGGSGGGGSGGGGSGGGGSGGGGSGGGGSGGGGSGGGGS

SEQ ID NO: 14 - protein, 12 aa, synthetic construct
  REGION 1..12: note = First linker peptide 1-10
GAGGGGAGGGGA

SEQ ID NO: 15 - protein, 42 aa, synthetic construct
  REGION 1..42: note = First linker peptide 1-11
GAGGGGAGGGGAGGGGAGGGGAGGGGAGGGGAGGGGAGGGGA

SEQ ID NO: 16 - protein, 15 aa, synthetic construct
  REGION 1..15: note = First linker peptide 1-12
GGGGAGGGGAGGGGA

SEQ ID NO: 17 - protein, 20 aa, synthetic construct
  REGION 1..20: note = First linker peptide 1-13
GGGGAGGGGAGGGGAGGGGA

SEQ ID NO: 18 - protein, 25 aa, synthetic construct
  REGION 1..25: note = First linker peptide 1-14
GGGGAGGGGAGGGGAGGGGAGGGGA

SEQ ID NO: 19 - protein, 30 aa, synthetic construct
  REGION 1..30: note = First linker peptide 1-15
GGGGAGGGGAGGGGAGGGGAGGGGAGGGGA

SEQ ID NO: 20 - protein, 40 aa, synthetic construct
  REGION 1..40: note = First linker peptide 1-16
GGGGAGGGGAGGGGAGGGGAGGGGAGGGGAGGGGAGGGGA

SEQ ID NO: 21 - protein, 20 aa, synthetic construct
  REGION 1..20: note = First linker peptide 1-17
GGGGGGGGGGGGGGGGGGGG

SEQ ID NO: 22 - protein, 25 aa, synthetic construct
  REGION 1..25: note = First linker peptide 1-18
GGGGGGGGGGGGGGGGGGGGGGGGG

SEQ ID NO: 23 - protein, 15 aa, synthetic construct
  REGION 1..15: note = First linker peptide 1-19
GGGGSGGGGSGGGGS

SEQ ID NO: 24 - protein, 25 aa, synthetic construct
  REGION 1..25: note = First linker peptide 1-20
GGGGSGGGGSGGGGSGGGGSGGGGS

SEQ ID NO: 25 - protein, 30 aa, synthetic construct
  REGION 1..30: note = First linker peptide 1-21
GGGGSGGGGSGGGGSGGGGSGGGGSGGGGS

SEQ ID NO: 26 - protein, 585 aa, synthetic construct
  REGION 1..585: note = HSA(C34S)
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 27 - protein, 585 aa, synthetic construct
  REGION 1..585: note = GSA(C34S)
DAHKSEVAHRFKDLGEETFKALVLVAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAARYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALAELVKHKPKATKEQLKTVMDDFAAFVEKCCKADDKETCFAEEGKKLVAASQAALGL

SEQ ID NO: 28 - protein, 20 aa, synthetic construct
  REGION 1..20: note = Second linker peptide 2-1
APAPAPAPAPAPAPAPAPAP

SEQ ID NO: 29 - protein, 42 aa, synthetic construct; identical to SEQ ID NO: 15
  REGION 1..42: note = Second linker peptide 2-2

SEQ ID NO: 30 - protein, 42 aa, synthetic construct
  REGION 1..42: note = Second linker peptide 2-3
GSGGGGSGGGGSGGGGSGGGGSGGGGSGGGGSGGGGSGGGGS

SEQ ID NO: 31 - protein, 108 aa, synthetic construct
  REGION 1..108: note = GDF15 variant 1
DHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 32 - protein, 112 aa, synthetic construct
  REGION 1..112: note = GDF15 WT mature
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 33 - protein, 768 aa, synthetic construct
  REGION 1..768: note = GLP1-GDF15 Fusion Protein 1
HGEGTFTSDVSSYLEEQAAKEFIAWLVKGGGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 34 - protein, 758 aa, synthetic construct
  REGION 1..758: note = GLP1-GDF15 Fusion Protein 2
HGEGTFTSDVSSYLEEQAAKEFIAWLVKGGGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 35 - protein, 793 aa, synthetic construct
  REGION 1..793: note = GLP1-GDF15 Fusion Protein 3
HGEGTFTSDVSSYLEEQAAKEFIAWLVKGGAPAPAPAPAPAPAPAPAPAPAPAPAPAPAPAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 36 - protein, 783 aa, synthetic construct
  REGION 1..783: note = GLP1-GDF15 Fusion Protein 4
HGEGTFTSDVSSYLEEQAAKEFIAWLVKGGAPAPAPAPAPAPAPAPAPAPAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 37 - protein, 773 aa, synthetic construct
  REGION 1..773: note = GLP1-GDF15 Fusion Protein 5
HGEGTFTSDVSSYLEEQAAKEFIAWLVKGGAPAPAPAPAPAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 38 - protein, 783 aa, synthetic construct
  REGION 1..783: note = GLP1-GDF15 Fusion Protein 6
HGEGTFTSDVSSYLEEQAAKEFIAWLVKGGGGGGAGGGGAGGGGAGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 39 - protein, 802 aa, synthetic construct
  REGION 1..802: note = GLP1-GDF15 Fusion Protein 7
HGEGTFTSDLSKQMEEEAVRLFIEWLKNGGPSSGAPPPSAPAPAPAPAPAPAPAPAPAPAPAPAPAPAPAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 40 - protein, 782 aa, synthetic construct
  REGION 1..782: note = GLP1-GDF15 Fusion Protein 8
HGEGTFTSDLSKQMEEEAVRLFIEWLKNGGPSSGAPPPSAPAPAPAPAPAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 41 - protein, 772 aa, synthetic construct
  REGION 1..772: note = GLP1-GDF15 Fusion Protein 9
HGEGTFTSDLSKQMEEEAVRLFIEWLKNGGPSSGAPPPSAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 42 - protein, 792 aa, synthetic construct
  REGION 1..792: note = GLP1-GDF15 Fusion Protein 10
HGEGTFTSDLSKQMEEEAVRLFIEWLKNGGPSSGAPPPSGGGGAGGGGAGGGGAGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 43 - protein, 777 aa, synthetic construct
  REGION 1..777: note = GLP1-GDF15 Fusion Protein 11
HGEGTFTSDLSKQMEEEAVRLFIEWLKNGGPSSGAPPPSGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 44 - protein, 762 aa, synthetic construct
  REGION 1..762: note = GLP1-GDF15 Fusion Protein 12
HGEGTFTSDLSKQMEEEAVRLFIEWLKNGGPSSGAPPPSAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 45 - protein, 763 aa, synthetic construct
  REGION 1..763: note = GLP1-GDF15 Fusion Protein 13
HGEGTFTSDVSSYLEEQAAKEFIAWLVKGGAPAPAPAPAPAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 46 - protein, 784 aa, synthetic construct
  REGION 1..784: note = GLP1-GDF15 Fusion Protein 14
HGEGTFTSDLSKQMEEEAVRLFIEWLKNGGPSSGAPPPSAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 47 - protein, 790 aa, synthetic construct
  REGION 1..790: note = GLP1-GDF15 Fusion Protein 15
HGEGTFTSDVSSYLEEQAAKEFIAWLVKGGGGGGGGGGGGGGGGGGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 48 - protein, 785 aa, synthetic construct
  REGION 1..785: note = GLP1-GDF15 Fusion Protein 16
HGEGTFTSDVSSYLEEQAAKEFIAWLVKGGGGGGGGGGGGGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 49 - protein, 785 aa, synthetic construct
  REGION 1..785: note = GLP1-GDF15 Fusion Protein 17
HGEGTFTSDVSSYLEEQAAKEFIAWLVKGGGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 50 - protein, 790 aa, synthetic construct
  REGION 1..790: note = GLP1-GDF15 Fusion Protein 18
HGEGTFTSDVSSYLEEQAAKEFIAWLVKGGGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 51 - protein, 807 aa, synthetic construct
  REGION 1..807: note = GLP1-GDF15 Fusion Protein 19
HGEGTFTSDVSSYLEEQAAKEFIAWLVKGGGAGGGGAGGGGAGGGGAGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 52 - protein, 786 aa, synthetic construct
  REGION 1..786: note = GLP1-GDF15 Fusion Protein 20
HGEGTFTSDLSKQMEEEAVRLFIEWLKNGGPSSGAPPPSGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 53 - protein, 795 aa, synthetic construct
  REGION 1..795: note = GLP1-GDF15 Fusion Protein 21
HGEGTFTSDVSSYLEEQAAKEFIAWLVKGGGGGGAGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 54 - protein, 790 aa, synthetic construct
  REGION 1..790: note = GLP1-GDF15 Fusion Protein 22
HGEGTFTSDVSSYLEEQAAKEFIAWLVKGGGGGGAGGGGAGGGGAGGGGAGGGGADAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 55 - protein, 795 aa, synthetic construct
  REGION 1..795: note = GLP1-GDF15 Fusion Protein 23
HGEGTFTSDVSSYLEEQAAKEFIAWLVKGGGGGGSGGGGSGGGGSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 56 - protein, 790 aa, synthetic construct
  REGION 1..790: note = GLP1-GDF15 Fusion Protein 24
HGEGTFTSDVSSYLEEQAAKEFIAWLVKGGGGGGSGGGGSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 57 - protein, 789 aa, synthetic construct
  REGION 1..789: note = GLP1-GDF15 Fusion Protein 25
HGEGTFTSDVSSYLEEQAAKEFIAWLVKGGASAPAPAPAPAPAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 58 - protein, 807 aa, synthetic construct
  REGION 1..807: note = GLP1-GDF15 Fusion Protein 26
HGEGTFTSDVSSYLEEQAAKEFIAWLVKGGASGGGGSGGGGSGGGGSGGGGSGGGGSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 59 - protein, 816 aa, synthetic construct
  REGION 1..816: note = GLP1-GDF15 Fusion Protein 27
HGEGTFTSDLSKQMEEEAVRLFIEWLKNGGPSSGAPPPSASGGGGSGGGGSGGGGSGGGGSGGGGSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 60 - protein, 798 aa, synthetic construct
  REGION 1..798: note = GLP1-GDF15 Fusion Protein 28
HGEGTFTSDLSKQMEEEAVRLFIEWLKNGGPSSGAPPPSASAPAPAPAPAPAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 61 - protein, 789 aa, synthetic construct
  REGION 1..789: note = GLP1-GDF15 Fusion Protein 29
HSEGTFTSDVSSYLEGQAAKEFIEWLVKGRASAPAPAPAPAPAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 62 - protein, 807 aa, synthetic construct
  REGION 1..807: note = GLP1-GDF15 Fusion Protein 30
HSEGTFTSDVSSYLEGQAAKEFIEWLVKGRASGGGGSGGGGSGGGGSGGGGSGGGGSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 63 - protein, 780 aa, synthetic construct
  REGION 1..780: note = GLP1-GDF15 Fusion Protein 31
HGEGTFTSDVSSYLEEQAAKEFIAWLVKGGGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 64 - protein, 789 aa, synthetic construct
  REGION 1..789: note = GLP1-GDF15 Fusion Protein 32
HGEGTFTSDLSKQMEEEAVRLFIEWLKNGGPSSGAPPPSGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 65 - protein, 777 aa, synthetic construct
  REGION 1..777: note = GLP1-GDF15 Fusion Protein 33
HSEGTFTSDVSSYLEGQAAKEFIEWLVKGRASGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 66 - protein, 779 aa, synthetic construct
  REGION 1..779: note = GLP1-GDF15 Fusion Protein 34
HSEGTFTSDVSSYLEGQAAKEFIEWLVKGRASAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 67 - protein, 786 aa, synthetic construct
  REGION 1..786: note = GLP1-GDF15 Fusion Protein 35
HGEGTFTSDLSKQMEEEAVRLFIEWLKNGGPSSGAPPPSASGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 68 - protein, 788 aa, synthetic construct
  REGION 1..788: note = GLP1-GDF15 Fusion Protein 36
HGEGTFTSDLSKQMEEEAVRLFIEWLKNGGPSSGAPPPSASAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 69 - protein, 777 aa, synthetic construct
  REGION 1..777: note = GLP1-GDF15 Fusion Protein 37
HGEGTFTSDVSSYLEEQAAKEFIAWLVKGGASGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 70 - protein, 779 aa, synthetic construct
  REGION 1..779: note = GLP1-GDF15 Fusion Protein 38
HGEGTFTSDVSSYLEEQAAKEFIAWLVKGGASAPAPAPAPAPGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 71 - protein, 780 aa, synthetic construct
  REGION 1..780: note = GLP1-GDF15 Fusion Protein 39
HSEGTFTSDVSSYLEGQAAKEFIEWLVKGRGGGGSGGGGSGGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 72 - protein, 780 aa, synthetic construct
  REGION 1..780: note = GLP1-GDF15 Fusion Protein 40
HGEGTFTSDVSSYLEEQAAKEFIAWLVKGGGGGGSGGGGSGGGGSDAHKSEVAHRFKDLGEETFKALVLVAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAARYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALAELVKHKPKATKEQLKTVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 73 - protein, 780 aa, synthetic construct
  REGION 1..780: note = GLP1-GDF15 Fusion Protein 41
HGEGTFTSDVSSYLEEQAAKEFIAWLVKGGGGGGSGGGGSGGGGSDAHKSEVAHRFKDLGEETFKALVLVAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAARYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALAELVKHKPKATKEQLKTVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLRQKTDTGVSLQTYDDLLAKDCHCI

SEQ ID NO: 74 - protein, 778 aa, synthetic construct
  REGION 1..778: note = GLP1-GDF15 Fusion Protein 42
EGTFTSDVSSYLEEQAAKEFIAWLVKGGGGGGSGGGGSGGGGSDAHKSEVAHRFKDLGEETFKALVLVAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAARYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALAELVKHKPKATKEQLKTVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 75 - protein, 735 aa, synthetic construct
  REGION 1..735: note = GSA-GDF15 fusion protein
DAHKSEVAHRFKDLGEETFKALVLVAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAARYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALAELVKHKPKATKEQLKTVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 76 - protein, 20 aa, synthetic construct
  REGION 1..20: note = HSE peptide
HSEGTFTSDVSSYLEGQAAK

SEQ ID NO: 77 - protein, 20 aa, synthetic construct
  REGION 1..20: note = HGE-1 peptide
HGEGTFTSDVSSYLEEQAAK

SEQ ID NO: 78 - protein, 12 aa, synthetic construct
  REGION 1..12: note = HGE-2 peptide
HGEGTFTSDLSK

SEQ ID NO: 79 - protein, 21 aa, synthetic construct
  REGION 1..21: note = ALV peptide
ALVLIAFAQYLQQSPFEDHVK

SEQ ID NO: 80 - protein, 16 aa, synthetic construct
  REGION 1..16: note = TDT peptide
TDTGVSLQTYDDLLAK

SEQ ID NO: 81 - protein, 727 aa, synthetic construct
  REGION 1..727: note = HSA-GDF15 fusion protein
EFHHHHHHDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 82 - protein, 16 aa, synthetic construct
  REGION 1..16: note = VVS peptide
VVSVLTVLHQDWLNGK

SEQ ID NO: 83 - protein, 713 aa, synthetic construct
  REGION 1..713: note = HSA-GDF15 fusion peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 84 - protein, 762 aa, synthetic construct
  REGION 1..762: note = GLP1-GDF15 (I89R) fusion peptide
HGEGTFTSDLSKQMEEEAVRLFIEWLKNGGPSSGAPPPSAPAPAPAPAPDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLRQKTDTGVSLQTYDDLLAKDCHCI

(19) United States
(12) Patent Application Publication
(10) Pub. No.: US 2025/0197468 A1
(43) Pub. Date: Jun. 19, 2025

(54) GLUCAGON LIKE PEPTIDE 1 (GLP1)-GROWTH DIFFERENTIATION FACTOR 15 (GDF15) FUSION PROTEINS AND USES THEREOF

(71) Applicant: Janssen Sciences Ireland Unlimited Company, Cork (IE)

(72) Inventors: Shannon MULLICAN, Spring House, PA (US); Matthew M. RANKIN, Spring House, PA (US); Xiefan LIN-SCHMIDT, Spring House, PA (US); Chichi HUANG, Spring House, PA (US); Jennifer FURMAN, San Diego, CA (US); Songmao ZHENG, Spring House, PA (US); Shamina RANGWALA, Spring House, PA (US); Serena NELSON, San Diego, CA (US)

(21) Appl. No.: 19/072,966

(22) Filed: Mar. 6, 2025

Related U.S. Application Data
(63) Continuation of application No. 18/332,690, filed on Jun. 9, 2023, now Pat. No. 12,275,770.
Application No. 18/332,690 is a division of application No. 17/309,114, filed on Apr. 26, 2021, now Pat. No. 11,713,345.
(60) Provisional application No. 62/748,603, filed on Oct. 22, 2018.

(51) Int. Cl. C07K 14/605; A61K 38/00; C07K 14/475; C12N 15/63

(52) U.S. Cl. CPC C07K 14/605; C07K 14/475; C12N 15/63; A61K 38/00

(57) ABSTRACT

Provided herein are GLP1-GDF15 fusion proteins comprising a GLP1 or GLP1 variant peptide, a first linker peptide, a serum albumin protein, a second linker peptide, and a GDF15 protein.

### CROSS REFERENCE TO RELATED APPLICATIONS

This application is a Continuation Application of U.S. patent application Ser. No. 18/332,690, filed Jun. 9, 2023, which is a Divisional Application of U.S. patent application Ser. No. 17/309,114, filed Apr. 26, 2021, now issued U.S. Pat. No. 11,713,345, which is a Section 371 of International Application No. PCT/IB2019/059029, filed on Oct. 22, 2019, which was published in the English language on Apr. 30, 2020, under International Publication No. WO 2020/084496 A1, which claims priority to U.S. Provisional Application No. 62/748,603, filed Oct. 22, 2018. Each disclosure is incorporated herein by reference in its entirety.

### FIELD OF THE INVENTION

The present invention is directed generally to novel glucagon like peptide-1 (GLP1)-growth differentiation factor 15 (GDF15) fusion proteins. The GLP1-GDF15 fusion proteins modulate the GLP1R and/or the GDF15R. The invention also relates to pharmaceutical compositions and methods for use thereof. The novel GLP1-GDF15 fusion proteins are useful for preventing, treating or ameliorating diseases and disorders, such as obesity, type 2 diabetes, the metabolic syndrome, insulin resistance, and dyslipidemia, among others.

### REFERENCE TO AN ELECTRONIC SEQUENCE LISTING

The contents of the electronic sequence listing (200310_405C1_SeqListing.xml; Size: 141 kilobytes; and Date of Creation: Jun. 9, 2023) is herein incorporated by reference in its entirety.

### BACKGROUND OF THE INVENTION

GDF15, a member of the TGFβ family, is a secreted protein that circulates in plasma as a 25 kDa homodimer and elicits its biological function through interaction with the brainstem expressed receptor GFRAL (Mullican et al., Nat Med. 23:1150-7 (2017), Yang et al., Nat Med. 23:1158-66 (2017), Hsu et al., Nature 550:255-9 (2017), Emmerson et al., Nat Med 23:1215-9 (2017)). Plasma levels of GDF15 range between 150 and 1150 pg/ml in most individuals (Tsai et al., J Cachexia Sarcopenia Muscle 3:239-43 (2012)). Elevated plasma levels of GDF15 are associated with weight loss due to anorexia and cachexia in cancer, and in renal and heart failure. Furthermore, GDF15 is increased in patients experiencing weight loss after Roux-en-Y gastric bypass (RYGB) surgery (Vila et al., Clin Chem 57:309-16 (2011)).

The correlation between weight loss and GDF15 is conserved in rodents. Overexpression of GDF15 results in decreased food intake, lower body weight and protects mice from obesity, liver steatosis and glucose intolerance upon high fat diet feeding (Back et al., Gastroenterology 131:1553-60 (2006), Johnen et al., Nat Med 13:1333-40 (2007), Chrysovergis et al., Int J Obesity 38:1555-64 (2014), Macia et al., PloS One 7:e34868 (2012), Jones et al., Cell Reports 22:1522-30 (2018), Xiong et al., Sci Trans Med 9:412 (2017)). Xenografts of prostate tumor cells transfected with GDF15 also decrease food intake and body weight (Johnen et al., Nat Med 13:1333-40 (2007)). Conversely, numerous investigators have reported that mice lacking GDF15 gain more weight and have greater fat mass than wildtype animals (Strelau et al., J Neurosci 29:13640-8 (2009), Casanovas et al., Haematologica 98:444-7 (2013), Bonaterra et al., J Amer Heart Assoc 1:e002550 (2012), Tsai et al., PloS one. 8:e55174 (2013)).

The potential of pharmacologically administered GDF15 to decrease energy intake and thereby elicit weight loss has been demonstrated in mice, rats and monkeys. Lean mice treated with recombinant GDF15 eat less and lose weight (Johnen et al., Nat Med 13:1333-40 (2007), Hsu et al., Nature 550:255-9 (2017), Mullican et al., Nat Med 23:1150-7 (2017), Tsai et al., Int J Obesity 42:561-71 (2018)). Decreased food intake and body weight is also observed in genetic and diet induced rat and mouse models of obesity after administration of GDF15 (Johnen et al., Nat Med 13:1333-40 (2007), Hsu et al., Nature 550:255-9 (2017), Mullican et al., Nat Med 23:1150-7 (2017), Yang et al., Nat Med 23:1158-66 (2017), Tsai et al., Int J Obesity 42:561-71 (2018), Xiong et al., Sci Trans Med 9:412 (2017)). GDF15 treatment mediated weight loss in diet induced obese mice leads to metabolic improvements including enhanced glucose homeostasis and lower plasma triglycerides and cholesterol. These effects translate to higher species as a six-week daily treatment regimen with recombinant human GDF15 in spontaneously obese non-human primates reduced food intake, body weight, and plasma triglyceride concentrations and improved glucose tolerance (Xiong et al., Sci Trans Med 9:412 (2017)). Furthermore, the half-life of recombinant GDF15 was previously demonstrated to be extended by fusion to human serum albumin (HSA) and is predicted to be a therapeutic suitable for once-weekly dosing in human (Mullican et al., Nat Med 23:1150-7 (2017) and U.S. Patent Publication No. 2017/0327560.

GLP1 is a peptide hormone derived from the enteroendocrine cells of the gut that also reduces food intake leading to weight loss. This function is mediated by interaction with the GLP1 receptor (GLP1R) within the central nervous system and this ligand/receptor interaction in peripheral tissues has additional biological effects including enhancement of glucose stimulated insulin secretion, suppression of glucagon release and slowing of gastric emptying (Druker et al. Cell Met 27:740-56 (2018)). Harnessing all of these biological effects, GLP1R agonists improve glucose homeostasis and drive weight loss in humans. In addition, treatment with GLP1R agonists is reported to significantly improve cardiovascular outcomes in diabetic patients although the exact mechanism is yet to be determined (Lim et al. Trends Endocrinol Metab. 29:238-48 (2018)). GLP1R agonist therapeutics are peptide sequences based on either native human GLP1 or exendin-4 (a peptide isolated from the saliva of the Gila monster lizard) with modifications to prevent enzymatic cleavage. The GLP1R agonists can be delivered via platforms that extend half-life such as lipidation, antibody Fc or human serum albumin (HSA) (Cheang and Moyle, Chem Med Chem 13:662-71 (2018)).

Thus, it is desirable to obtain a GLP1 analogue or derivative thereof and/or a GDF15 analogue or derivative thereof with an improved metabolic stability and pharmacokinetic profile relative to GLP1 or GDF15, respectively. Such derivatives would provide GLP1 receptor and GDF15 receptor modulation with greater duration of action, making them suitable as therapeutic agents for subjects in need of such modulation.

The foregoing discussion is presented solely to provide a better understanding of the nature of the problems confronting the art and should not be construed in any way as an admission that such reference constitutes “prior art” to the instant application.

### SUMMARY OF THE INVENTION

In one general aspect, the invention relates to novel glucagon-like peptide-1 (GLP1)-growth differentiation factor 15 (GDF15) fusion proteins. The GLP1-GDF15 fusion proteins modulate the GLP1R and/or the GDF15R (GFRAL).

Provided herein are glucagon-like peptide-1 (GLP1)-growth differentiation factor 15 (GDF15) fusion proteins. The GLP1-GDF15 fusion proteins comprise a GLP1 peptide, a first linker peptide, a serum albumin protein, a second linker peptide, and a GDF15 protein.

In certain embodiments, GLP1 peptide comprises an amino acid sequence selected from the group consisting of SEQ ID NOs:1-4.

In certain embodiments, the first linker peptide comprises an amino acid sequence selected from the group consisting of SEQ ID NOs:5-25.

In certain embodiments, the serum albumin protein comprises an amino acid sequence selected from SEQ ID NO:26 or SEQ ID NO:27.

In certain embodiments, the second linker peptide comprises an amino acid sequence selected from the group consisting of SEQ ID NOs:28-30.

In certain embodiments, the GDF15 protein comprises an amino acid sequence selected from SEQ ID NO:31 or SEQ ID NO:32.

In certain embodiments, the GLP1-GDF15 fusion protein comprises an amino acid sequence selected from the group consisting of SEQ ID NOs:33-74 and 84.

Also provided are isolated nucleic acids encoding the GLP1-GDF15 fusion proteins of the invention. Also provided are vectors comprising the isolated nucleic acids of the invention. Also provided are host cells comprising the isolated nucleic acids of the invention or the vectors of the invention.

Also provided are pharmaceutical compositions comprising the GLP1-GDF15 fusion proteins of the invention and a pharmaceutically acceptable carrier.

Also provided are methods for treating or preventing a disease or disorder in a subject in need thereof, wherein said disease or disorder is selected from the group consisting of obesity, type I or type II diabetes, metabolic syndrome, insulin resistance, impaired glucose tolerance, hyperglycemia, hyperinsulinemia, hypertriglyceridemia, hypoglycemia due to congenital hyperinsulinism (CHI), dyslipidemia, atherosclerosis, diabetic nephropathy, and other cardiovascular risk factors such as hypertension and cardiovascular risk factors related to unmanaged cholesterol and/or lipid levels, osteoporosis, inflammation, non-alcoholic fatty liver disease (NAFLD), non-alcoholic steatohepatitis (NASH), renal disease, and eczema, the method comprising administering to the subject in need thereof an effective amount of the pharmaceutical composition of the invention.

Also provided are methods of reducing food intake in a subject in need thereof, the method comprising administering to the subject in need thereof an effective amount of the pharmaceutical composition of the invention.

Also provided are methods of modulating GLP1 receptor activity in a subject in need thereof, the method comprising administering to the subject in need thereof an effective amount of the pharmaceutical composition of the invention.

Also provided are methods of modulating GDF15 receptor (GFRAL) activity in a subject in need thereof, the method comprising administering to the subject in need thereof an effective amount of the pharmaceutical composition of the invention.

In certain embodiments, the pharmaceutical composition is administered via an injection.

Also provided are kits comprising the GLP1-GDF15 fusion proteins of the invention, the isolated nucleic acids of the invention, and/or the vectors of the invention. The kit can, for example, further comprise a device for injection.

Also provided are methods of producing a pharmaceutical composition comprising the GLP1-GDF15 fusion proteins of the invention. The methods comprise combining the GLP1-GDF15 fusion proteins with a pharmaceutically acceptable carrier to obtain the pharmaceutical composition.

Also provided are methods of producing the GLP1-GDF15 fusion proteins of the invention. The methods comprise culturing a cell comprising a nucleic acid encoding the GLP1-GDF15 fusion protein under conditions to produce the GLP1-GDF15 fusion protein and recovering the GLP1-GDF15 fusion protein from the cell or culture.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a graph demonstrating percent weight change (from day 0) in DIO mice receiving daily administration of liraglutide (lira) and/or every other day administration of HSA-GDF15 (SEQ ID NO:81).

FIG. 2 shows a graph demonstrating average percent weight change (from day 0) in DIO mice receiving daily administration of GLP1-GSA-GDF15 (SEQ ID NO:72), GLP1-GSA-GDF15(I89R) (SEQ ID NO:73, I89R mutation abolishes GDF15 activity), GLP1(9-39)-GSA-GDF15 (SEQ ID NO:74, abolishes GLP1 activity), or GSA-GDF15 (SEQ ID NO:75). Dulaglutide treatment served as a positive control, while vehicle treatment served as a negative control. ±SEM, n=8.

FIG. 3 shows a schematic of a GLP1-GDF15 fusion protein.

FIG. 4 shows a graph demonstrating the effects of subcutaneous administration of SEQ ID NOs:45, 49, 50, 33, 46, and 44 on food intake in C57BL/6 mice. Data is presented as percent change in food intake as compared to food intake prior to treatment at 24 hours.

FIG. 5 shows a graph demonstrating the plasma concentration of GLP1-GDF15 fusion proteins (SEQ ID NOs:45, 49, 50, 33, 46, and 44) at 24 hours after administration in mice as determined by immune-affinity capture-trypsin digestion-LC-MS/MS analysis.

FIG. 6 shows a graph demonstrating the effects of subcutaneous administration of SEQ ID NOs:45, 50, 46, and 44 on food intake in GFRAL-deficient mice. Data is presented as percent change in food intake (compared to vehicle treatment) versus the plasma concentration of the test article with intact GLP1 arms at 24 hours.

FIG. 7 shows a graph demonstrating the effects of subcutaneous administration of SEQ ID NO:45 on food intake in GFRAL-deficient mice. Data is presented as percent change in food intake (compared to vehicle treatment) versus the plasma concentration of the test article with intact GLP1 arms at 24 hours.

FIG. 8 shows a graph demonstrating the effects of subcutaneous administration of SEQ ID NO:33 on food intake in GFRAL-deficient mice. Data is presented as percent change in food intake (compared to vehicle treatment) versus the plasma concentration of the test article with intact GLP1 arms at 24 hours.

FIG. 9 shows a graph demonstrating the effects of subcutaneous administration of SEQ ID NOs:50, 46, and 44 on glucose tolerance in mice. Data is presented as percent change in delta AUC compared to vehicle treatment versus the plasma concentration of the GLP1-GDF15 fusion proteins with intact GLP1 peptides or GLP1 peptide variants.

FIG. 10 shows a graph demonstrating the effects of subcutaneous administration of SEQ ID NOs:45 and 49 on glucose tolerance in mice. Data is presented as percent change in delta AUC compared to vehicle treatment versus the plasma concentration of the GLP1-GDF15 fusion proteins with intact GLP1 peptides or GLP1 peptide variants.

FIG. 11 shows a graph demonstrating the effects of subcutaneous administration of SEQ ID NO:33 on glucose tolerance in mice. Data is presented as percent change in delta AUC compared to vehicle treatment versus the plasma concentration of the GLP1-GDF15 fusion proteins with intact GLP1 peptides or GLP1 peptide variants.

FIG. 12 shows a graph demonstrating the effects of subcutaneous administration of SEQ ID NO:45 and SEQ ID NO:36 on glucose tolerance in mice. Data is presented as percent change in delta AUC compared to vehicle treatment versus the plasma concentration of the GLP1-GDF15 fusion proteins with intact GLP1 peptides or GLP1 peptide variants.

FIG. 13 shows a graph demonstrating the effects of subcutaneous administration of SEQ ID NOs: 45 and 44 on insulin secretion during a graded glucose infusion in cynomolgus monkeys. Data is presented as fold change in ISR AUC normalized to glucose AUC (compared to baseline) versus the plasma concentration of the GLP1-GDF15 fusion proteins with intact GLP1 peptides or GLP1 peptide variants.

FIG. 14 shows a graph demonstrating the weight loss efficacy of daily subcutaneous administration of GLP1-GDF15 fusion proteins (SEQ ID NOs:45 and 44) in diet induced obese mice. Data is presented as percent change in body weight from Day 0.

FIG. 15 shows a graph demonstrating the plasma concentration of GLP1-GDF15 fusion proteins (SEQ ID NOs:45 and 44) after 7 days of daily administration in diet induced obese mice as determined by immuno-affinity capture-trypsin digestion-LC-MS/MS analysis.

FIG. 16 shows a graph demonstrating the 3-day percent change in food intake at baseline and after each QW subcutaneous administration of GLP1-GDF15 fusion proteins (SEQ ID NOs:44 and 83) over 4 weeks in spontaneously obese nonhuman primates. Data is presented for individual subjects and those without detectable level of test article in their plasma are not included in the figure as outlined below the x-axis.

FIG. 17 shows a graph demonstrating the plasma concentration of total test article during a 4-week treatment with varying concentrations of GLP1-GDF15 fusion proteins (SEQ ID NOs:44 and 83) administered QW in spontaneously obese nonhuman primates as determined by immunoassay. Data is presented as the average (±SEM).

FIG. 18 shows a graph demonstrating the plasma concentration of GLP1 moiety containing test article during a 4-week treatment with varying concentrations of GLP1-GDF15 fusion protein (SEQ ID NO. 44) administered QW in spontaneously obese nonhuman primates as determined by immunoassay. Data is presented as the average (±SEM).

FIG. 19 shows a graph demonstrating the absolute daily food intake before, during and after a 21-day treatment with escalating concentrations of GLP1-GDF15 fusion proteins (SEQ ID NOs: 44, 83 and 84) administered Q3D in spontaneously obese nonhuman primates. Data is presented as the average (±SEM) of ten subjects per group.

FIG. 20 shows a graph demonstrating body weight change (relative to baseline) after a 21-day treatment with escalating concentrations of GLP1-GDF15 fusion proteins (SEQ ID NOS: 44, 83 and 84) administered Q3D in spontaneously obese nonhuman primates. Data is presented as the average (±SEM) of ten subjects per group.

FIG. 21 shows a graph demonstrating the plasma concentration of total test article during and after a 21-day treatment with escalating concentrations of GLP1-GDF15 fusion proteins (SEQ ID NOs: 44, 83 and 84) administered Q3D in spontaneously obese nonhuman primates as determined by immunoassay. Data is presented for individual subjects.

FIG. 22 shows a graph demonstrating the plasma concentration of GLP1 moiety containing test article during and after a 21-day treatment with escalating concentrations of GLP1-GDF15 fusion proteins (SEQ ID NOs: 44 and 84) administered Q3D in spontaneously obese nonhuman primates as determined by immunoassay. Data is presented for individual subjects.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., GLP1 peptide, linker peptides, serum albumin proteins, GDF15 proteins and polynucleotides that encode the peptides), refer to two or more sequences or subsequences that are the same or have a specified percentage of amino acid residues or nucleotides that are the same, when compared and aligned for maximum correspondence, as measured using one of the following sequence comparison algorithms or by visual inspection using methods known in the art in view of the present disclosure.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F.M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The term “administering” with respect to the methods of the invention, means a method for therapeutically or prophylactically preventing, treating or ameliorating a syndrome, disorder or disease as described herein by using a conjugate or compound of the invention or a form, composition or medicament thereof. Such methods include administering an effective amount of said conjugate, compound, a form, composition or medicament thereof at different times during the course of a therapy or concurrently in a combination form. The methods of the invention are to be understood as embracing all known therapeutic treatment regimens.

The term “effective amount” means that amount of active conjugate, compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated.

As used herein, the term “composition” is intended to encompass a product comprising the specified ingredients in the specified amounts, as well as any product which results, directly or indirectly, from combinations of the specified ingredients in the specified amounts.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The convention one-letter or three-letter code for amino acid residues is used herein. The terms “peptide,” “polypeptide,” and “protein” can be used interchangeably herein to refer to polymers of amino acids of any length, including those comprising linked (e.g., fused) peptides/polypeptide (e.g., fusion proteins). The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Glucagon-Like Peptide-1 (GLP1)-Growth Differentiation Factor 15 (GDF15) Fusion Proteins

Although both GDF15 and GLP1 signaling can reduce food intake, these effects appear to be independent of one another. For example, GDF15 effects are maintained in the absence of GLP1R in mice and conversely, GLP1 treatment still leads to food intake effects in the absence of GFRAL (Hsu et al., Nature 550:255-9 (2017), Mullican et al., Nat Med 23:1150-7 (2017)). Therefore, it is hypothesized that targeting both mechanisms simultaneously could lead to greater food intake reductions and weight loss than either mechanism alone. Combining the independent yet complementary pharmacology of GDF15 and GLP1 agonists through fusion to human serum albumin will deliver benefits on weight loss, insulin sensitivity, insulin secretion and cardiovascular outcomes with a single fully recombinant molecule suitable for once-weekly administration. A major challenge to this approach will be to deliver each agonist within the molecule in a balanced manner that engages the corresponding receptor at the desired level, enough to achieve all desired effects but that avoids on-target adverse effects. This balance can be fine-tuned by adjusting potency and/or pharmacokinetic properties of either agonist arm of the molecule.

In one general aspect, the invention relates to glucagon-like peptide-1 (GLP1)-growth differentiation factor 15 (GDF15) fusion proteins. The GLP1-GDF15 fusion proteins comprise a GLP1 or GLP1 variant peptide, a first linker peptide (e.g., an amino (N)-terminal linker), a human serum albumin (HSA) protein, a second linker peptide (e.g., a carboxy (C)-terminal linker), and a GDF15 or GDF15 variant protein.

### Glucagon-Like Peptide-1 (GLP1) or GLP1 Variant Peptide

Glucagon-like peptide 1 (GLP1) is an insulin secretagogue synthesized in the intestine and released in response to the intake of food. It is secreted primarily in two forms, GLP1-(7-37) and GLP1-(7-36)NH₂, both of which bind to a specific GLP1 receptor (GLP1R) found in many tissues including the pancreatic beta-cell where it augments glucose-stimulated insulin secretion and in the brainstem where it controls satiety and meal size.

Numerous GLP1 analogs and derivatives are known and can be referred to herein as “GLP1 variants.” These GLP1 variant peptides can include the Exendins, which are peptides found in the venom of the Gila monster. These Exendins have sequence homology to native GLP1 and can bind the GLP1 receptor and initiate the signal transduction cascade response.

GLP1 and GLP1 variant peptides have been shown to act in a variety of manners, which can include, but are not limited to, decreasing food intake, stimulating insulin release, lowering glucagon secretion, inhibiting gastric emptying, and enhancing glucose utilization.

GLP1R belongs to the class B family of 7-transmembrane-spanning, heterotrimeric G-protein-coupled receptors and is expressed in a wide range of tissues including, but not limited to, α-, β-, and δ-cells of the pancreatic islets, heart, kidney, stomach, intestine, nodose ganglion neurons of the vagus nerve, and several regions of the central nervous system (CNS) including the hypothalamus and brainstem. The GLP1R can couple to Gαₛ, Gα_{q}, Gαᵢ, and Gαₒ (Montrose-Rafizadeh et al., Endocrinology 140:1132-40 (1999); Hallbrink et al., Biochim Biophys Acta 1546:79-86 (2001)) leading to increases in intracellular calcium, adenylate cyclase, and phospholipase C, and activation of PKA, PKC, PI-3K, Epac2, and MAPK signal transduction pathways (Drucker et al., PNAS 84:3434-8 (1987); Wheeler et al., Endrocrinology 133:57-62 (1993); and Holz et al., JBC 270:17749-57 (1995)).

Provided herein are GLP1-GDF15 fusion proteins that comprise a first component, wherein the first component is a GLP1 or GLP1 variant peptide. As used herein, the terms “GLP1 peptide,” “GLP1 variant peptide,” “GLP1 peptide variant,” and “GLP1 or GLP1 variant peptide” are used interchangeably. The GLP1 or GLP1 variant peptide can comprise one of the sequences provided in Table 1. The GLP1 or GLP1 variant peptide can comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to one of the sequences provided in Table 1. The GLP1 or GLP1 variant peptide sequence can be chosen based on at least one of the following criteria: (i) expression yield and purity, (ii) in vitro stability, (iii) in vitro potency, (iv) the retention of in vitro potency in combination with the GDF15 or GDF15 variant protein, (v) lack of serine xylosylation or potential for serine xylosylation, and (vi) properties of the GLP1-GDF15 fusion proteins (e.g., in vivo stability, in vivo potency (i.e., whether the GLP1 or GLP1 variant peptide and GDF15 or GDF15 variant protein are capable of having agonist activity on GLP1R and GDF15R (GFRAL) receptor, respectively)).

The GLP1 or GLP1 variant peptides that make up the first component of the GLP1 fusion peptide are intended to encompass peptides that have sufficient homology and functionality to the native GLP1. The GLP1 or GLP1 variant peptides are designed to be capable of binding to the GLP1 receptor in tissues including the pancreas and brainstem resulting in the same signaling pathway and exhibiting the same or similar physiological activity as when the native GLP1 binds the GLP1 receptor in these tissues.

TABLE 1
Glucagon-like peptide-1
and variants thereof

| GLP1 or GLP1 | | SEQ |
|---|---|---|
| Variant | | ID |
| Peptide | Peptide Sequence | NO: |
| (A8S, A30E) | HSEGTFTSDVSSYLEG | 1 |
| GLP1(7-36) | QAAKEFIEWLVKGR | |
| (A8G, G22E, R36G) | HGEGTFTSDVSSYLEE | 2 |
| GLP1 (7-36) | QAAKEFIAWLVKGG | |
| Exendin 4 (1-39) | HGEGTFTSDLSKQMEE | 3 |
| | EAVRLFIEWLKNGGPS | |
| | SGAPPPS | |
| Exendin 4 (1-28) | HGEGTFTSDLSKQMEE | 4 |
| | EAVRLFIEWLKN | |

### First Linker Peptide: Amino-Terminal Liker (N-Terminal Linker)

Provided herein are GLP1-GDF15 fusion proteins that comprise a second component, wherein the second component is a first linker peptide (i.e., an amino-terminal linker peptide). The first linker peptide can comprise one of the sequences provided in Table 2. The first linker peptide can comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to one of the sequences provided in Table 2.

The first linker peptide can, for example, comprise about 5 to about 60 amino acid residues, about 10 to about 50 amino acid residues, about 10 to about 60 amino acid residues, about 5 to about 50 amino acid residues, about 15 to about 40 amino acid residues, about 12 to about 30 amino acid residues, about 12 to about 42 amino acid residues, about 20 to about 25 amino acid residues, about 8 to about 48 amino acid residues, about 10 to about 46 amino acid residues, about 12 to about 44 amino acid residues, about 14 to about 42 amino acid residues, about 16 to about 40 amino acid residues, about 18 to about 38 amino acid residues, about 20 to about 36 amino acid residues, about 20 to about 42 amino acid residues, about 22 to about 34 amino acid residues, about 24 to about 32 amino acid residues, about 26 to about 30 amino acid residues, or any value in between. The first linker peptide can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acid residues.

In certain embodiments, the first linker peptide can contain an alanine-proline repeat (i.e., an AP repeat), wherein an AP dipeptide can be referred to as an AP unit. The AP repeat can comprise 2, 3, 4, 5, 6, 7, 8, 9, 10, 11, 12, 13, 14, 15, 16, 17, 18, 19, 20, 21, 22, 23, 24, 25, 26, or 27 AP units. In certain embodiments, the first linker peptide can, for example, comprise 2 to 25 AP units, 5 to 25 units, 4 to 23 AP units, 6 to 21 AP units, 8 to 19 AP units, 10 to 17 AP units, 12 to 15 AP units, 5 to 10 AP units, 5 to 15 AP units, 10 to 25 AP units, 15 to 25 AP units, 20 to 25 AP units, or any value in between. In certain embodiments, the AP repeat can be internal to an alanine-serine dipeptide (i.e., an AS unit) and a glycine-serine dipeptide (i.e., a GS unit). The AS unit can, for example, be at the amino terminal end of the first linker peptide. The GS unit can, for example, be at the carboxyl terminal end of the first linker peptide.

In certain embodiments, the first linker peptide can contain a glycine-glycine-glycine-glycine-serine repeat (i.e., a G₄S repeat), wherein a G₄S pentapeptide can be referred to as a G₄S unit. The GAS repeat can comprise 2, 3, 4, 5, 6, 7, 8, 9, 10, 11, 12, 13, 14, or 15 G₄S units. In certain embodiments, the first linker peptide can, for example, comprise 2 to 15 G₄S units, 4 to 13 G₄S units, 6 to 11 G₄S units, 8 to 9 G₄S units, 2 to 8 G₄S units, 2 to 6 G₄S units, 6 to 8 G₄S units, 7 to 8 G₄S units, 7 to 9 G₄S units, 7 to 10 G₄S units, or any value in between. In certain embodiments, an AS or a GS unit can, for example be at the amino terminal end of the first linker peptide.

In certain embodiments, the first linker peptide can contain a glycine-glycine-glycine-glycine-alanine repeat (i.e., a G₄A repeat), wherein a G₄A pentapeptide can be referred to as a G₄A unit. The G₄A repeat can comprise 2, 3, 4, 5, 6, 7, 8, 9, 10, 11, 12, 13, 14, or 15 G₄A units. In certain embodiments, the first linker peptide can, for example, comprise 2 to 15 G₄A units, 4 to 13 G₄A units, 6 to 11 G₄A units, 8 to 9 G₄A units, 2 to 8 G₄A units, 2 to 4 G₄A units, 2 to 6 G₄A units, 4 to 6 G₄A units, 4 to 8 G₄A units, 6 to 8 G₄A units, 6 to 9 G₄A units, 6 to 10 G₄A units or any value in between. In certain embodiments, a glycine-alanine dipeptide (i.e., a GA unit) unit can, for example be at the amino terminal end of the first linker peptide.

In certain embodiments, the first linker peptide can be a poly-glycine peptide. The poly-glycine peptide can comprise about 6 to about 50 glycine residues, about 10 to about 45 glycine residues, about 15 to about 40 glycine residues, about 20 to about 35 glycine residues, about 25 to about 30 glycine residues, about 20 to about 25 glycine residues, or any number in between. The poly-glycine first linker peptide can comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 glycine residues.

The first linker peptide sequence can be chosen based on at least one of the following criteria: (i) expression yield and purity, (ii) in vitro potency, (iii) in vitro stability, (iv) lack of serine xylosylation or potential for serine xylosylation, and (v) properties of the GLP1-GDF15 fusion protein (e.g., in vivo stability, in vivo potency (i.e., whether GLP1 or GLP1 variant peptide and GDF15 or GDF15 variant protein are capable of having agonist activity on GLP1R and GDF15R (GFRAL), respectively)).

TABLE 2
First linker peptides (N-terminal linker peptide)

| First Linker | | SEQ ID |
|---|---|---|
| Peptide | Peptide Sequence | NO: |
| 1-1 | APAPAPAPAP | 5 |
| 1-2 | APAPAPAPAPAPAPAPAPAP | 6 |
| 1-3 | APAPAPAPAPAPAPAPAPAPAPAPAPAPAP | 7 |
| 1-4 | APAPAPAPAPAPAPAPAPAPAPAPAPAPA | 8 |
| | PAPAPAPAPAP | |
| 1-5 | APAPAPAPAPAPAPAPAPAPAPAPAPAPA | 9 |
| | PAPAPAPAPAPAPAPAPAPAP | |
| 1-6 | ASAPAPAPAPAPAPAPAPAPAPGS | 10 |
| 1-7 | ASAPAPAPAPAPGS | 11 |
| 1-8 | ASGGGGSGGGGS | 12 |
| 1-9 | ASGGGGSGGGGSGGGGSGGGGSGGGGSGGG | 13 |
| | GSGGGGSGGGGS | |
| 1-10 | GAGGGGAGGGGA | 14 |
| 1-11 | GAGGGGAGGGGAGGGGAGGGGAGGGGAGG | 15 |
| | GGAGGGGAGGGGA | |
| 1-12 | GGGGAGGGGAGGGGA | 16 |
| 1-13 | GGGGAGGGGAGGGGAGGGGA | 17 |
| 1-14 | GGGGAGGGGAGGGGAGGGGAGGGGA | 18 |
| 1-15 | GGGGAGGGGAGGGGAGGGGAGGGGAGGGGA | 19 |
| 1-16 | GGGGAGGGGAGGGGAGGGGAGGGGAGGGG | 20 |
| | AGGGGAGGGGA | |
| 1-17 | GGGGGGGGGGGGGGGGGGGG | 21 |
| 1-18 | GGGGGGGGGGGGGGGGGGGGGGGGG | 22 |
| 1-19 | GGGGSGGGGSGGGGS | 23 |
| 1-20 | GGGGSGGGGSGGGGSGGGGSGGGGS | 24 |
| 1-21 | GGGGSGGGGSGGGGSGGGGSGGGGSGGGGS | 25 |

### Serum Albumin Protein

Provided herein are GLP1-GDF15 fusion proteins that comprise a third component, wherein the third component is a serum albumin protein (e.g., a human serum albumin (HSA) protein or a gorilla serum albumin (GSA) protein). Native human serum albumin protein contains 35 cysteine (Cys, C) residues that form 17 disulfide bonds, with the Cys-34 residue being the only free cysteine in the molecule. This free Cys-34 has been shown to function as a free radical scavenger, by trapping multiple reactive oxygen species (ROS) and reactive nitrogen species (RNS) (Taverna et al., Ann. Intensive Care 3:4 (2013)). This free Cys was mutated to serine (Ser) to minimize the risk of heterogeneity due to oxidation.

The serum albumin protein can comprise one of the sequences provided in Table 3. The serum albumin protein can comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to one of the sequences provided in Table 3. The serum albumin protein sequence can be chosen based on at least one of the following criteria: (i) in vitro stability, (ii) in vitro potency, and (iii) properties of the GLP1-GDF15 fusion protein (e.g., in vivo stability and in vivo potency (i.e., whether the GLP1 or GLP1 variant peptide and GDF15 or GDF15 variant protein are capable of having agonist activity on GLP1R and GDF15R, respectively)).

TABLE 3
Serum Albumin protein

| Half-life | | SEQ |
|---|---|---|
| extension | | ID |
| protein | Protein Sequence | NO: |
| HSA | DAHKSEVAHRFKDLGEENFKALVLIAFAQY | 26 |
| (C34S) | LQQSPFEDHVKLVNEVTEFAKTCVADESAE | |
| | NCDKSLHTLFGDKLCTVATLRETYGEMADC | |
| | CAKQEPERNECFLQHKDDNPNLPRLVRPEV | |
| | DVMCTAFHDNEETFLKKYLYEIARRHPYFY | |
| | APELLFFAKRYKAAFTECCQAADKAACLLP | |
| | KLDELRDEGKASSAKQRLKCASLQKFGERA | |
| | FKAWAVARLSQRFPKAEFAEVSKLVTDLTK | |
| | VHTECCHGDLLECADDRADLAKYICENQDS | |
| | ISSKLKECCEKPLLEKSHCIAEVENDEMPA | |
| | DLPSLAADFVESKDVCKNYAEAKDVFLGMF | |
| | LYEYARRHPDYSVVLLLRLAKTYETTLEKC | |
| | CAAADPHECYAKVFDEFKPLVEEPQNLIKQ | |
| | NCELFEQLGEYKFQNALLVRYTKKVPQVST | |
| | PTLVEVSRNLGKVGSKCCKHPEAKRMPCAE | |
| | DYLSVVLNQLCVLHEKTPVSDRVTKCCTES | |
| | LVNRRPCFSALEVDETYVPKEFNAETFTFH | |
| | ADICTLSEKERQIKKQTALVELVKHKPKAT | |
| | KEQLKAVMDDFAAFVEKCCKADDKETCFAE | |
| | EGKKLVAASQAALGL | |
| GSA | DAHKSEVAHRFKDLGEETFKALVLVAFAQY | 27 |
| (C34S) | LQQSPFEDHVKLVNEVTEFAKTCVADESAE | |
| | NCDKSLHTLFGDKLCTVATLRETYGEMADC | |
| | CAKQEPERNECFLQHKDDNPNLPRLVRPEV | |
| | DVMCTAFHDNEETFLKKYLYEIARRHPYFY | |
| | APELLFFAARYKAAFTECCQAADKAACLLP | |
| | KLDELRDEGKASSAKQRLKCASLQKFGERA | |
| | FKAWAVARLSQRFPKAEFAEVSKLVTDLTK | |
| | VHTECCHGDLLECADDRADLAKYICENQDS | |
| | ISSKLKECCEKPLLEKSHCLAEVENDEMPA | |
| | DLPSLAADFVESKDVCKNYAEAKDVFLGMF | |
| | LYEYARRHPDYSVVLLLRLAKTYETTLEKC | |
| | CAAADPHECYAKVFDEFKPLVEEPQNLIKQ | |
| | NCELFEQLGEYKFQNALLVRYTKKVPQVST | |
| | PTLVEVSRNLGKVGSKCCKHPEAKRMPCAE | |
| | DYLSVVLNQLCVLHEKTPVSDRVTKCCTES | |
| | LVNRRPCFSALEVDETYVPKEFNAETFTFH | |
| | ADICTLSEKERQIKKQTALAELVKHKPKAT | |
| | KEQLKTVMDDFAAFVEKCCKADDKETCFAE | |
| | EGKKLVAASQAALGL | |

### Second Linker Peptide: Carboxy-Terminal Linker (C-Terminal Linker)

Provided herein are GLP1-GDF15 fusion proteins that comprise a fourth component, wherein the fourth component is a second linker peptide (i.e., a carboxy-terminal linker peptide). The second linker peptide can comprise one of the sequences provided in Table 4. The second linker peptide can comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to one of the sequences provided in Table 4.

The second linker peptide can, for example, comprise about 5 to about 60 amino acid residues, about 10 to about 50 amino acid residues, about 10 to about 60 amino acid residues, about 5 to about 50 amino acid residues, about 15 to about 40 amino acid residues, about 12 to about 30 amino acid residues, about 12 to about 42 amino acid residues, about 20 to about 25 amino acid residues, about 8 to about 48 amino acid residues, about 10 to about 46 amino acid residues, about 12 to about 44 amino acid residues, about 14 to about 42 amino acid residues, about 16 to about 40 amino acid residues, about 18 to about 38 amino acid residues, about 20 to about 36 amino acid residues, about 20 to about 42 amino acid residues, about 22 to about 34 amino acid residues, about 24 to about 32 amino acid residues, about 26 to about 30 amino acid residues, or any value in between. The second linker peptide can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 amino acid residues.

In certain embodiments, the second linker peptide can contain an alanine-proline repeat (i.e., an AP repeat), wherein an AP dipeptide can be referred to as an AP unit. The AP repeat can comprise 2, 3, 4, 5, 6, 7, 8, 9, 10, 11, 12, 13, 14, 15, 16, 17, 18, 19, 20, 21, 22, 23, 24, 25, 26 or 27 AP units. In certain embodiments, the second linker peptide can, for example, comprise 2 to 25 AP units, 5 to 25 units, 4 to 23 AP units, 6 to 21 AP units, 8 to 19 AP units, 10 to 17 AP units, 12 to 15 AP units, 5 to 10 units, 5 to 15 units, 10 to 25 units, 15 to 25 units, 20 to 25 units, or any value in between. In certain embodiments, the AP repeat can be internal to an alanine-serine dipeptide (i.e., an AS unit) and a glycine-serine dipeptide (i.e., a GS unit). The AS unit can, for example, be at the amino terminal end of the second linker peptide. The GS unit can, for example, be at the carboxyl terminal end of the second linker peptide.

In certain embodiments, the second linker peptide can contain a glycine-glycine-glycine-glycine-serine repeat (i.e., a G₄S repeat), wherein a G₄S pentapeptide can be referred to as a G₄S unit. The G₄S repeat can comprise 2, 3, 4, 5, 6, 7, 8, 9, 10, 11, 12, 13, 14, or 15 G₄S units. In certain embodiments, the second linker peptide can, for example, comprise 2 to 15 G₄S units, 4 to 13 G₄S units, 6 to 11 G₄S units, 8 to 9 G₄S units, 2 to 8 G₄S units, 2 to 6 G₄S units, 6 to 8 G₄S units, 7 to 8 G₄S units, 7 to 9 G₄S units, 7 to 10 G₄S units, or any value in between. In certain embodiments, an AS or a GS unit can, for example be at the amino terminal end of the second linker peptide.

In certain embodiments, the second linker peptide can contain a glycine-glycine-glycine-glycine-alanine repeat (i.e., a G₄A repeat), wherein a G₄A pentapeptide can be referred to as a G₄A unit. The G₄A repeat can comprise 2, 3, 4, 5, 6, 7, 8, 9, 10, 11, 12, 13, 14, or 15 G₄A units. In certain embodiments, the second linker peptide can, for example, comprise 2 to 15 G₄A units, 4 to 13 G₄A units, 6 to 11 G₄A units, 8 to 9 G₄A units, 2 to 8 G₄A units, 2 to 4 G₄A units, 2 to 6 G₄A units, 4 to 6 G₄A units, 4 to 8 G₄A units, 6 to 8 G₄A units, 6 to 9 G₄A units, 6 to 10 G₄A units, or any value in between. In certain embodiments, a glycine-alanine dipeptide (i.e., a GA unit) unit can, for example be at the amino terminal end of the second linker peptide.

In certain embodiments, the second linker peptide can be a poly-glycine peptide. The poly-glycine peptide can comprise about 6 to about 50 glycine residues, about 10 to about 45 glycine residues, about 15 to about 40 glycine residues, about 20 to about 35 glycine residues, about 25 to about 30 glycine residues, about 20 to about 25 glycine residues, or any number in between. The poly-glycine second linker peptide can comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 glycine residues.

The second linker peptide sequence can be chosen based on at least one of the following criteria: (i) expression yield and purity, (ii) in vitro potency, (iii) in vitro stability, (iv) lack of serine xylosylation or potential for serine xylosylation, and (v) properties of the GLP1-GDF15 fusion protein (e.g., in vivo stability, in vivo potency (i.e., whether the GLP1 or GLP1 variant peptide and GDF15 or GDF15 variant protein are capable of having agonist activity of GLP1R and GDF15R (GFRAL), respectively)).

TABLE 4
Second linker peptide
(C-terminal linker peptide)

| Second linker | | SEQ ID |
|---|---|---|
| peptide | Peptide Sequence | NO: |
| 2-1 | APAPAPAPAPAPAPAPAPAP | 28 |
| 2-2 | GAGGGGAGGGGAGGGGAGGGG | 29 |
| | AGGGGAGGGGAGGGGAGGGGA | |
| 2-3 | GSGGGGSGGGGSGGGGSGGGG | 30 |
| | SGGGGSGGGGSGGGGSGGGGS | |

### GDF15 or GDF15 Variant Protein

Growth differentiation factor 15 (GDF15) is a protein belonging to the transforming growth factor beta (TGF-β) superfamily. GDF15 is a secreted protein that circulates as a 25-kDa dimer. GDF15 is also referred to as prostate derived factor (PDF), macrophage inhibitory cytokine-1 (MIC-1), NSAID (nonsteroidal anti-inflammatory drugs)-activated gene (NAG-1) and placental TGF-beta (PTGFβ).

GDF15 function has yet to be fully elucidated, but has been implicated in multiple biological processes including, but not limited to, energy homeostasis, body weight regulation, and cachexia driven by cancer and chronic disease. The potential of pharmacologically administered GDF15 to decrease energy intake and thereby elicit weight loss has been demonstrated in mice, rats and monkeys. (Johnen et al., Nat Med 13:1333-40 (2007), Hsu et al., Nature 550:255-9 (2017), Mullican et al., Nat Med 23:1150-7 (2017), Tsai et al., Int J Obesity 42:561-71 (2018)). GDF15 treatment mediated weight loss leads to metabolic improvements including enhanced glucose homeostasis and lower plasma triglycerides and cholesterol (Xiong et al., Sci Trans Med 9:412 (2017)).

GDF15 binds to GDNF Family Receptor Alpha Like (GFRAL), a transmembrane receptor exclusively located in neurons in the brainstem (Mullican et al., Hsu et al, Yang et al., Emmerson et al.). Upon GDF15 binding, GFRAL complexes with RET, a tyrosine kinase that stimulates a downstream intracellular phosphorylation cascade including the post-translational modification of AKT, ERK and PLCγ. While the additional molecular and cellular components of this cascade remain to be elucidated, the ultimate effect of GDF15/GFRAL signaling is decreased food intake and weight loss (Mullican and Rangwala, 2018).

Provided herein are GLP1-GDF15 fusion proteins that comprise a fifth component, wherein the fifth component is a GDF15 or GDF15 variant protein. The GDF15 protein can comprise the sequence provided in Table 5, and a GDF15 variant protein can comprise a variant of the sequence provided in Table 5. The GDF15 or GDF15 variant protein can comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to one of the sequences provided in Table 5. The GDF15 and/or GDF15 variant protein sequence can be chosen based on at least one of the following criteria: (i) expression yield and purity, (ii) in vitro stability, (iii) in vitro potency, (iv) the retention of in vitro potency in combination with the GLP1 or GLP1 variant protein, (v) lack of serine xylosylation or potential for serine xylosylation, and (vi) properties of the GLP1-GDF15 fusion protein (e.g., in vivo stability, in vivo potency (i.e., whether GLP1 or GLP1 variant peptide and GDF15 or GDF15 variant protein are capable of having agonist activity on GLP1R and/or GDF15R (GFRAL) receptor, respectively)).

The GDF15 or GDF15 variant proteins that make up the fifth component of the GLP1-GDF15 fusion proteins are intended to encompass proteins that have sufficient homology and functionality to activate the native GDF15R (GFRAL). The GDF15 or GDF15 variant proteins are designed to be capable of binding to GFRAL in the brainstem resulting in the same signaling pathway and exhibiting the same impact on food intake as when the native GDF15 binds GFRAL in these neurons.

TABLE 5
GDF15 or GDF15 variant protein

| GDF15 or GDF15 | | SEQ |
|---|---|---|
| variant | | ID |
| protein | Protein Sequence | NO: |
| GDF15 variant 1 | DHCPLGPGRCCRLHTVRASLEDLGW | 31 |
| | ADWVLSPREVQVTMCIGACPSQFRA | |
| | ANMHAQIKTSLHRLKPDTVPAPCCV | |
| | PASYNPMVLIQKTDTGVSLQTYD | |
| | DLLAKDCHCI | |
| GDF15 WT | ARNGDHCPLGPGRCCRLHTVRASLED | 32 |
| mature | LGWADWVLSPREVQVTMCIGACPSQF | |
| | RAANMHAQIKTSLHRLKPDTVPAPCC | |
| | VPASYNPMVLIQKTDTGVSLQ | |
| | TYDDLLAKDCHCI | |

### GLP1-GDF15 Fusion Proteins

Provided herein are GLP1-GDF15 fusion proteins that comprise a first, a second, a third, a fourth, and a fifth component as described previously. The first component is a GLP1 or GLP1 variant peptide, the second component is a first linker peptide, the third component is a serum albumin protein, the fourth component is a second linker peptide, and the fifth component is a GDF15 or GDF15 variant protein. The GLP1-GDF15 fusion proteins can comprise one of the sequences provided in Table 6. The GLP1-GDF15 fusion protein can comprise at least 85%, 86%, 87%, 88%, 89%, 90%, 91%, 92%, 93%, 94%, 95%, 96%, 97%, 98%, or 99% identity to one of the sequences provided in Table 6. The GLP1-GDF15 fusion protein can be chosen based on at least one of the following criteria: (i) expression yield and purity, (ii) in vitro potency, (iii) in vitro stability, (iv) lack of serine xylosylation, and (v) physical properties of the GLP1-GDF15 fusion protein (e.g., in vivo stability, in vivo potency (i.e., whether the GLP1 or GLP1 variant peptide and GDF15 or GDF15 variant protein are capable of having agonist activity on GLP1R and GDF15R, respectively)), and (vi) desired balance of dual agonism pharmacology in vivo.

For a fusion protein therapeutic that delivers dual pharmacology in one molecule, it is important to properly adjust the pharmacokinetic/pharmacodynamic (PK/PD) properties of each moiety so that both agonists are in the therapeutic range with the intended dosing. Extensive investigation of various glucagon-like peptide-1 (GLP1)-growth differentiation factor 15 (GDF15) fusion proteins comprising various combinations of a GLP1 peptide or GLP1 variant peptide sequence, a first linker peptide sequence, a serum albumin protein sequence, a second linker peptide sequence, and a GDF15 or GDF15 variant protein sequence resulted in the discovery of novel molecules possessing the unique property of delivering optimal balanced doses of both GLP1 and GDF15 agonists. These novel GLP1-GDF15 fusion proteins were demonstrated to be as efficacious in engaging the GDF15R (GFRAL) in vivo as HSA-GDF15, when used at a specific dose range (see Example 11). Furthermore, the GLP1-GDF15 fusion proteins, used at the same dose range (see Example 11), were unexpectedly as efficacious in engaging the GLP1R as dulaglutide (GLP1-Fc), and without adverse side effects, despite having a GLP1 moiety exposure 10-30 times greater than that in dulaglutide. The delivery of GLP1 peptides as fusions with HSA-GDF15 unexpectedly altered the in vivo potency of the GLP1 moiety yet enabled novel glucagon-like peptide-1 (GLP1)-growth differentiation factor 15 (GDF15) fusion proteins to have an optimal balance of both agonists.

TABLE 6
GLP1-GDF15 fusion proteins

| GLP1- | | |
|---|---|---|
| GDF15 | | SEQ |
| fusion | | ID |
| proteins | Protein Sequence | NO: |
| 1 | HGEGTFTSDVSSYLEEQAAKEFIAW | 33 |
| | LVKGGGGGGAGGGGAGGGGAGGGGA | |
| | GGGGADAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLAPAPAPAPAP | |
| | APAPAPAPAPDHCPLGPGRCCRLHT | |
| | VRASLEDLGWADWVLSPREVQVTMC | |
| | IGACPSQFRAANMHAQIKTSLHRLK | |
| | PDTVPAPCCVPASYNPMVLIQKTDT | |
| | GVSLQTYDDLLAKDCHCI | |
| 2 | HGEGTFTSDVSSYLEEQAAKEFIAW | 34 |
| | LVKGGGGGGAGGGGAGGGGADAHKS | |
| | EVAHRFKDLGEENFKALVLIAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAKRYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCIAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALV | |
| | ELVKHKPKATKEQLKAVMDDFAAFV | |
| | EKCCKADDKETCFAEEGKKLVAASQ | |
| | AALGLAPAPAPAPAPAPAPAPAPAP | |
| | DHCPLGPGRCCRLHTVRASLEDLGW | |
| | ADWVLSPREVQVTMCIGACPSQFRA | |
| | ANMHAQIKTSLHRLKPDTVPAPCCV | |
| | PASYNPMVLIQKTDTGVSLQTYDDL | |
| | LAKDCHCI | |
| 3 | HGEGTFTSDVSSYLEEQAAKEFIAW | 35 |
| | LVKGGAPAPAPAPAPAPAPAPAPAP | |
| | APAPAPAPAPAPAPAPAPAPAPAPA | |
| | PAPAPDAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLAPAPAPAPAP | |
| | APAPAPAPAPDHCPLGPGRCCRLHT | |
| | VRASLEDLGWADWVLSPREVQVTMC | |
| | IGACPSQFRAANMHAQIKTSLHRLK | |
| | PDTVPAPCCVPASYNPMVLIQKTDT | |
| | GVSLQTYDDLLAKDCHCI | |
| 4 | HGEGTFTSDVSSYLEEQAAKEFIAW | 36 |
| | LVKGGAPAPAPAPAPAPAPAPAPAP | |
| | APAPAPAPAPAPAPAPAPAPDAHKS | |
| | EVAHRFKDLGEENFKALVLIAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAKRYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCIAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALV | |
| | ELVKHKPKATKEQLKAVMDDFAAFV | |
| | EKCCKADDKETCFAEEGKKLVAASQ | |
| | AALGLAPAPAPAPAPAPAPAPAPAP | |
| | DHCPLGPGRCCRLHTVRASLEDLGW | |
| | ADWVLSPREVQVTMCIGACPSQFRA | |
| | ANMHAQIKTSLHRLKPDTVPAPCCV | |
| | PASYNPMVLIQKTDTGVSLQTYDDL | |
| | LAKDCHCI | |
| 5 | HGEGTFTSDVSSYLEEQAAKEFIAW | 37 |
| | LVKGGAPAPAPAPAPAPAPAPAPAP | |
| | APAPAPAPAPDAHKSEVAHRFKDLG | |
| | EENFKALVLIAFAQYLQQSPFEDHV | |
| | KLVNEVTEFAKTCVADESAENCDKS | |
| | LHTLFGDKLCTVATLRETYGEMADC | |
| | CAKQEPERNECFLQHKDDNPNLPRL | |
| | VRPEVDVMCTAFHDNEETFLKKYLY | |
| | EIARRHPYFYAPELLFFAKRYKAAF | |
| | TECCQAADKAACLLPKLDELRDEGK | |
| | ASSAKQRLKCASLQKFGERAFKAWA | |
| | VARLSQRFPKAEFAEVSKLVTDLTK | |
| | VHTECCHGDLLECADDRADLAKYIC | |
| | ENQDSISSKLKECCEKPLLEKSHCI | |
| | AEVENDEMPADLPSLAADFVESKDV | |
| | CKNYAEAKDVFLGMFLYEYARRHPD | |
| | YSVVLLLRLAKTYETTLEKCCAAAD | |
| | PHECYAKVFDEFKPLVEEPQNLIKQ | |
| | NCELFEQLGEYKFQNALLVRYTKKV | |
| | PQVSTPTLVEVSRNLGKVGSKCCKH | |
| | PEAKRMPCAEDYLSVVLNQLCVLHE | |
| | KTPVSDRVTKCCTESLVNRRPCFSA | |
| | LEVDETYVPKEFNAETFTFHADICT | |
| | LSEKERQIKKQTALVELVKHKPKAT | |
| | KEQLKAVMDDFAAFVEKCCKADDKE | |
| | TCFAEEGKKLVAASQAALGLAPAPA | |
| | PAPAPAPAPAPAPAPDHCPLGPGRC | |
| | CRLHTVRASLEDLGWADWVLSPREV | |
| | QVTMCIGACPSQFRAANMHAQIKTS | |
| | LHRLKPDTVPAPCCVPASYNPMVLI | |
| | QKTDTGVSLQTYDDLLAKDCHCI | |
| 6 | HGEGTFTSDVSSYLEEQAAKEFIAW | 38 |
| | LVKGGGGGGAGGGGAGGGGAGGGGA | |
| | GGGGAGGGGAGGGGAGGGGADAHKS | |
| | EVAHRFKDLGEENFKALVLIAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAKRYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCIAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALV | |
| | ELVKHKPKATKEQLKAVMDDFAAFV | |
| | EKCCKADDKETCFAEEGKKLVAASQ | |
| | AALGLAPAPAPAPAPAPAPAPAPAP | |
| | DHCPLGPGRCCRLHTVRASLEDLGW | |
| | ADWVLSPREVQVTMCIGACPSQFRA | |
| | ANMHAQIKTSLHRLKPDTVPAPCCV | |
| | PASYNPMVLIQKTDTGVSLQTYDDL | |
| | LAKDCHCI | |
| 7 | HGEGTFTSDLSKQMEEEAVRLFIEW | 39 |
| | LKNGGPSSGAPPPSAPAPAPAPAPA | |
| | PAPAPAPAPAPAPAPAPAPAPAPAP | |
| | APAPAPAPAPAPAPDAHKSEVAHRF | |
| | KDLGEENFKALVLIAFAQYLQQSPF | |
| | EDHVKLVNEVTEFAKTCVADESAEN | |
| | CDKSLHTLFGDKLCTVATLRETYGE | |
| | MADCCAKQEPERNECFLQHKDDNPN | |
| | LPRLVRPEVDVMCTAFHDNEETFLK | |
| | KYLYEIARRHPYFYAPELLFFAKRY | |
| | KAAFTECCQAADKAACLLPKLDELR | |
| | DEGKASSAKQRLKCASLQKFGERAF | |
| | KAWAVARLSQRFPKAEFAEVSKLVT | |
| | DLTKVHTECCHGDLLECADDRADLA | |
| | KYICENQDSISSKLKECCEKPLLEK | |
| | SHCIAEVENDEMPADLPSLAADFVE | |
| | SKDVCKNYAEAKDVFLGMFLYEYAR | |
| | RHPDYSVVLLLRLAKTYETTLEKCC | |
| | AAADPHECYAKVFDEFKPLVEEPQN | |
| | LIKQNCELFEQLGEYKFQNALLVRY | |
| | TKKVPQVSTPTLVEVSRNLGKVGSK | |
| | CCKHPEAKRMPCAEDYLSVVLNQLC | |
| | VLHEKTPVSDRVTKCCTESLVNRRP | |
| | CFSALEVDETYVPKEFNAETFTFHA | |
| | DICTLSEKERQIKKQTALVELVKHK | |
| | PKATKEQLKAVMDDFAAFVEKCCKA | |
| | DDKETCFAEEGKKLVAASQAALGLA | |
| | PAPAPAPAPAPAPAPAPAPDHCPLG | |
| | PGRCCRLHTVRASLEDLGWADWVLS | |
| | PREVQVTMCIGACPSQFRAANMHAQ | |
| | IKTSLHRLKPDTVPAPCCVPASYNP | |
| | MVLIQKTDTGVSLQTYDDLLAKDCH | |
| | CI | |
| 8 | HGEGTFTSDLSKQMEEEAVRLFIEW | 40 |
| | LKNGGPSSGAPPPSAPAPAPAPAPA | |
| | PAPAPAPAPAPAPAPAPAPDAHKSE | |
| | VAHRFKDLGEENFKALVLIAFAQYL | |
| | QQSPFEDHVKLVNEVTEFAKTCVAD | |
| | ESAENCDKSLHTLFGDKLCTVATLR | |
| | ETYGEMADCCAKQEPERNECFLQHK | |
| | DDNPNLPRLVRPEVDVMCTAFHDNE | |
| | ETFLKKYLYEIARRHPYFYAPELLF | |
| | FAKRYKAAFTECCQAADKAACLLPK | |
| | LDELRDEGKASSAKQRLKCASLQKF | |
| | GERAFKAWAVARLSQRFPKAEFAEV | |
| | SKLVTDLTKVHTECCHGDLLECADD | |
| | RADLAKYICENQDSISSKLKECCEK | |
| | PLLEKSHCIAEVENDEMPADLPSLA | |
| | ADFVESKDVCKNYAEAKDVFLGMFL | |
| | YEYARRHPDYSVVLLLRLAKTYETT | |
| | LEKCCAAADPHECYAKVFDEFKPLV | |
| | EEPQNLIKQNCELFEQLGEYKFQNA | |
| | LLVRYTKKVPQVSTPTLVEVSRNLG | |
| | KVGSKCCKHPEAKRMPCAEDYLSVV | |
| | LNQLCVLHEKTPVSDRVTKCCTESL | |
| | VNRRPCFSALEVDETYVPKEFNAET | |
| | FTFHADICTLSEKERQIKKQTALVE | |
| | LVKHKPKATKEQLKAVMDDFAAFVE | |
| | KCCKADDKETCFAEEGKKLVAASQA | |
| | ALGLAPAPAPAPAPAPAPAPAPAPD | |
| | HCPLGPGRCCRLHTVRASLEDLGWA | |
| | DWVLSPREVQVTMCIGACPSQFRAA | |
| | NMHAQIKTSLHRLKPDTVPAPCCVP | |
| | ASYNPMVLIQKTDTGVSLQTYDDLL | |
| | AKDCHCI | |
| 9 | HGEGTFTSDLSKQMEEEAVRLFIEW | 41 |
| | LKNGGPSSGAPPPSAPAPAPAPAPA | |
| | PAPAPAPAPDAHKSEVAHRFKDLGE | |
| | ENFKALVLIAFAQYLQQSPFEDHVK | |
| | LVNEVTEFAKTCVADESAENCDKSL | |
| | HTLFGDKLCTVATLRETYGEMADCC | |
| | AKQEPERNECFLQHKDDNPNLPRLV | |
| | RPEVDVMCTAFHDNEETFLKKYLYE | |
| | IARRHPYFYAPELLFFAKRYKAAFT | |
| | ECCQAADKAACLLPKLDELRDEGKA | |
| | SSAKQRLKCASLQKFGERAFKAWAV | |
| | ARLSQRFPKAEFAEVSKLVTDLTKV | |
| | HTECCHGDLLECADDRADLAKYICE | |
| | NQDSISSKLKECCEKPLLEKSHCIA | |
| | EVENDEMPADLPSLAADFVESKDVC | |
| | KNYAEAKDVFLGMFLYEYARRHPDY | |
| | SVVLLLRLAKTYETTLEKCCAAADP | |
| | HECYAKVFDEFKPLVEEPQNLIKQN | |
| | CELFEQLGEYKFQNALLVRYTKKVP | |
| | QVSTPTLVEVSRNLGKVGSKCCKHP | |
| | EAKRMPCAEDYLSVVLNQLCVLHEK | |
| | TPVSDRVTKCCTESLVNRRPCFSAL | |
| | EVDETYVPKEFNAETFTFHADICTL | |
| | SEKERQIKKQTALVELVKHKPKATK | |
| | EQLKAVMDDFAAFVEKCCKADDKET | |
| | CFAEEGKKLVAASQAALGLAPAPAP | |
| | APAPAPAPAPAPAPDHCPLGPGRCC | |
| | RLHTVRASLEDLGWADWVLSPREVQ | |
| | VTMCIGACPSQFRAANMHAQIKTSL | |
| | HRLKPDTVPAPCCVPASYNPMVLIQ | |
| | KTDTGVSLQTYDDLLAKDCHCI | |
| 10 | HGEGTFTSDLSKQMEEEAVRLFIEW | 42 |
| | LKNGGPSSGAPPPSGGGGAGGGGAG | |
| | GGGAGGGGAGGGGAGGGGAGGGGAG | |
| | GGGADAHKSEVAHRFKDLGEENFKA | |
| | LVLIAFAQYLQQSPFEDHVKLVNEV | |
| | TEFAKTCVADESAENCDKSLHTLFG | |
| | DKLCTVATLRETYGEMADCCAKQEP | |
| | ERNECFLQHKDDNPNLPRLVRPEVD | |
| | VMCTAFHDNEETFLKKYLYEIARRH | |
| | PYFYAPELLFFAKRYKAAFTECCQA | |
| | ADKAACLLPKLDELRDEGKASSAKQ | |
| | RLKCASLQKFGERAFKAWAVARLSQ | |
| | RFPKAEFAEVSKLVTDLTKVHTECC | |
| | HGDLLECADDRADLAKYICENQDSI | |
| | SSKLKECCEKPLLEKSHCIAEVEND | |
| | EMPADLPSLAADFVESKDVCKNYAE | |
| | AKDVFLGMFLYEYARRHPDYSVVLL | |
| | LRLAKTYETTLEKCCAAADPHECYA | |
| | KVFDEFKPLVEEPQNLIKQNCELFE | |
| | QLGEYKFQNALLVRYTKKVPQVSTP | |
| | TLVEVSRNLGKVGSKCCKHPEAKRM | |
| | PCAEDYLSVVLNQLCVLHEKTPVSD | |
| | RVTKCCTESLVNRRPCFSALEVDET | |
| | YVPKEFNAETFTFHADICTLSEKER | |
| | QIKKQTALVELVKHKPKATKEQLKA | |
| | VMDDFAAFVEKCCKADDKETCFAEE | |
| | GKKLVAASQAALGLAPAPAPAPAPA | |
| | PAPAPAPAPDHCPLGPGRCCRLHTV | |
| | RASLEDLGWADWVLSPREVQVTMCI | |
| | GACPSQFRAANMHAQIKTSLHRLKP | |
| | DTVPAPCCVPASYNPMVLIQKTDTG | |
| | VSLQTYDDLLAKDCHCI | |
| 11 | HGEGTFTSDLSKQMEEEAVRLFIEW | 43 |
| | LKNGGPSSGAPPPSGGGGAGGGGAG | |
| | GGGAGGGGAGGGGADAHKSEVAHRF | |
| | KDLGEENFKALVLIAFAQYLQQSPF | |
| | EDHVKLVNEVTEFAKTCVADESAEN | |
| | CDKSLHTLFGDKLCTVATLRETYGE | |
| | MADCCAKQEPERNECFLQHKDDNPN | |
| | LPRLVRPEVDVMCTAFHDNEETFLK | |
| | KYLYEIARRHPYFYAPELLFFAKRY | |
| | KAAFTECCQAADKAACLLPKLDELR | |
| | DEGKASSAKQRLKCASLQKFGERAF | |
| | KAWAVARLSQRFPKAEFAEVSKLVT | |
| | DLTKVHTECCHGDLLECADDRADLA | |
| | KYICENQDSISSKLKECCEKPLLEK | |
| | SHCIAEVENDEMPADLPSLAADFVE | |
| | SKDVCKNYAEAKDVFLGMFLYEYAR | |
| | RHPDYSVVLLLRLAKTYETTLEKCC | |
| | AAADPHECYAKVFDEFKPLVEEPQN | |
| | LIKQNCELFEQLGEYKFQNALLVRY | |
| | TKKVPQVSTPTLVEVSRNLGKVGSK | |
| | CCKHPEAKRMPCAEDYLSVVLNQLC | |
| | VLHEKTPVSDRVTKCCTESLVNRRP | |
| | CFSALEVDETYVPKEFNAETFTFHA | |
| | DICTLSEKERQIKKQTALVELVKHK | |
| | PKATKEQLKAVMDDFAAFVEKCCKA | |
| | DDKETCFAEEGKKLVAASQAALGLA | |
| | PAPAPAPAPAPAPAPAPAPDHCPLG | |
| | PGRCCRLHTVRASLEDLGWADWVLS | |
| | PREVQVTMCIGACPSQFRAANMHAQ | |
| | IKTSLHRLKPDTVPAPCCVPASYNP | |
| | MVLIQKTDTGVSLQTYDDLLAKDCH | |
| | CI | |
| 12 | HGEGTFTSDLSKQMEEEAVRLFIEW | 44 |
| | LKNGGPSSGAPPPSAPAPAPAPAPD | |
| | AHKSEVAHRFKDLGEENFKALVLIA | |
| | FAQYLQQSPFEDHVKLVNEVTEFAK | |
| | TCVADESAENCDKSLHTLFGDKLCT | |
| | VATLRETYGEMADCCAKQEPERNEC | |
| | FLQHKDDNPNLPRLVRPEVDVMCTA | |
| | FHDNEETFLKKYLYEIARRHPYFYA | |
| | PELLFFAKRYKAAFTECCQAADKAA | |
| | CLLPKLDELRDEGKASSAKQRLKCA | |
| | SLQKFGERAFKAWAVARLSQRFPKA | |
| | EFAEVSKLVTDLTKVHTECCHGDLL | |
| | ECADDRADLAKYICENQDSISSKLK | |
| | ECCEKPLLEKSHCIAEVENDEMPAD | |
| | LPSLAADFVESKDVCKNYAEAKDVF | |
| | LGMFLYEYARRHPDYSVVLLLRLAK | |
| | TYETTLEKCCAAADPHECYAKVFDE | |
| | FKPLVEEPQNLIKQNCELFEQLGEY | |
| | KFQNALLVRYTKKVPQVSTPTLVEV | |
| | SRNLGKVGSKCCKHPEAKRMPCAED | |
| | YLSVVLNQLCVLHEKTPVSDRVTKC | |
| | CTESLVNRRPCFSALEVDETYVPKE | |
| | FNAETFTFHADICTLSEKERQIKKQ | |
| | TALVELVKHKPKATKEQLKAVMDDF | |
| | AAFVEKCCKADDKETCFAEEGKKLV | |
| | AASQAALGLAPAPAPAPAPAPAPAP | |
| | APAPDHCPLGPGRCCRLHTVRASLE | |
| | DLGWADWVLSPREVQVTMCIGACPS | |
| | QFRAANMHAQIKTSLHRLKPDTVPA | |
| | PCCVPASYNPMVLIQKTDTGVSLQT | |
| | YDDLLAKDCHCI | |
| 13 | HGEGTFTSDVSSYLEEQAAKEFIAW | 45 |
| | LVKGGAPAPAPAPAPAPAPAPAPAP | |
| | DAHKSEVAHRFKDLGEENFKALVLI | |
| | AFAQYLQQSPFEDHVKLVNEVTEFA | |
| | KTCVADESAENCDKSLHTLFGDKLC | |
| | TVATLRETYGEMADCCAKQEPERNE | |
| | CFLQHKDDNPNLPRLVRPEVDVMCT | |
| | AFHDNEETFLKKYLYEIARRHPYFY | |
| | APELLFFAKRYKAAFTECCQAADKA | |
| | ACLLPKLDELRDEGKASSAKQRLKC | |
| | ASLQKFGERAFKAWAVARLSQRFPK | |
| | AEFAEVSKLVTDLTKVHTECCHGDL | |
| | LECADDRADLAKYICENQDSISSKL | |
| | KECCEKPLLEKSHCIAEVENDEMPA | |
| | DLPSLAADFVESKDVCKNYAEAKDV | |
| | FLGMFLYEYARRHPDYSVVLLLRLA | |
| | KTYETTLEKCCAAADPHECYAKVFD | |
| | EFKPLVEEPQNLIKQNCELFEQLGE | |
| | YKFQNALLVRYTKKVPQVSTPTLVE | |
| | VSRNLGKVGSKCCKHPEAKRMPCAE | |
| | DYLSVVLNQLCVLHEKTPVSDRVTK | |
| | CCTESLVNRRPCFSALEVDETYVPK | |
| | EFNAETFTFHADICTLSEKERQIKK | |
| | QTALVELVKHKPKATKEQLKAVMDD | |
| | FAAFVEKCCKADDKETCFAEEGKKL | |
| | VAASQAALGLAPAPAPAPAPAPAPA | |
| | PAPAPDHCPLGPGRCCRLHTVRASL | |
| | EDLGWADWVLSPREVQVTMCIGACP | |
| | SQFRAANMHAQIKTSLHRLKPDTVP | |
| | APCCVPASYNPMVLIQKTDTGVSLQ | |
| | TYDDLLAKDCHCI | |
| 14 | HGEGTFTSDLSKQMEEEAVRLFIEW | 46 |
| | LKNGGPSSGAPPPSAPAPAPAPAPD | |
| | AHKSEVAHRFKDLGEENFKALVLIA | |
| | FAQYLQQSPFEDHVKLVNEVTEFAK | |
| | TCVADESAENCDKSLHTLFGDKLCT | |
| | VATLRETYGEMADCCAKQEPERNEC | |
| | FLQHKDDNPNLPRLVRPEVDVMCTA | |
| | FHDNEETFLKKYLYEIARRHPYFYA | |
| | PELLFFAKRYKAAFTECCQAADKAA | |
| | CLLPKLDELRDEGKASSAKQRLKCA | |
| | SLQKFGERAFKAWAVARLSQRFPKA | |
| | EFAEVSKLVTDLTKVHTECCHGDLL | |
| | ECADDRADLAKYICENQDSISSKLK | |
| | ECCEKPLLEKSHCIAEVENDEMPAD | |
| | LPSLAADFVESKDVCKNYAEAKDVF | |
| | LGMFLYEYARRHPDYSVVLLLRLAK | |
| | TYETTLEKCCAAADPHECYAKVFDE | |
| | FKPLVEEPQNLIKQNCELFEQLGEY | |
| | KFQNALLVRYTKKVPQVSTPTLVEV | |
| | SRNLGKVGSKCCKHPEAKRMPCAED | |
| | YLSVVLNQLCVLHEKTPVSDRVTKC | |
| | CTESLVNRRPCFSALEVDETYVPKE | |
| | FNAETFTFHADICTLSEKERQIKKQ | |
| | TALVELVKHKPKATKEQLKAVMDDF | |
| | AAFVEKCCKADDKETCFAEEGKKLV | |
| | AASQAALGLGAGGGGAGGGGAGGGG | |
| | AGGGGAGGGGAGGGGAGGGGAGGGG | |
| | ADHCPLGPGRCCRLHTVRASLEDLG | |
| | WADWVLSPREVQVTMCIGACPSQFR | |
| | AANMHAQIKTSLHRLKPDTVPAPCC | |
| | VPASYNPMVLIQKTDTGVSLQTYDD | |
| | LLAKDCHCI | |
| 15 | HGEGTFTSDVSSYLEEQAAKEFIAW | 47 |
| | LVKGGGGGGGGGGGGGGGGGGGGGG | |
| | GGGGGDAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLGAGGGGAGGG | |
| | GAGGGGAGGGGAGGGGAGGGGAGGG | |
| | GAGGGGADHCPLGPGRCCRLHTVRA | |
| | SLEDLGWADWVLSPREVQVTMCIGA | |
| | CPSQFRAANMHAQIKTSLHRLKPDT | |
| | VPAPCCVPASYNPMVLIQKTDTGVS | |
| | LQTYDDLLAKDCHCI | |
| 16 | HGEGTFTSDVSSYLEEQAAKEFIAW | 18 |
| | LVKGGGGGGGGGGGGGGGGGGGGGG | |
| | DAHKSEVAHRFKDLGEENFKALVLI | |
| | AFAQYLQQSPFEDHVKLVNEVTEFA | |
| | KTCVADESAENCDKSLHTLFGDKLC | |
| | TVATLRETYGEMADCCAKQEPERNE | |
| | CFLQHKDDNPNLPRLVRPEVDVMCT | |
| | AFHDNEETFLKKYLYEIARRHPYFY | |
| | APELLFFAKRYKAAFTECCQAADKA | |
| | ACLLPKLDELRDEGKASSAKQRLKC | |
| | ASLQKFGERAFKAWAVARLSQRFPK | |
| | AEFAEVSKLVTDLTKVHTECCHGDL | |
| | LECADDRADLAKYICENQDSISSKL | |
| | KECCEKPLLEKSHCIAEVENDEMPA | |
| | DLPSLAADFVESKDVCKNYAEAKDV | |
| | FLGMFLYEYARRHPDYSVVLLLRLA | |
| | KTYETTLEKCCAAADPHECYAKVFD | |
| | EFKPLVEEPQNLIKQNCELFEQLGE | |
| | YKFQNALLVRYTKKVPQVSTPTLVE | |
| | VSRNLGKVGSKCCKHPEAKRMPCAE | |
| | DYLSVVLNQLCVLHEKTPVSDRVTK | |
| | CCTESLVNRRPCFSALEVDETYVPK | |
| | EFNAETFTFHADICTLSEKERQIKK | |
| | QTALVELVKHKPKATKEQLKAVMDD | |
| | FAAFVEKCCKADDKETCFAEEGKKL | |
| | VAASQAALGLGAGGGGAGGGGAGGG | |
| | GAGGGGAGGGGAGGGGAGGGGAGGG | |
| | GADHCPLGPGRCCRLHTVRASLEDL | |
| | GWADWVLSPREVQVTMCIGACPSQF | |
| | RAANMHAQIKTSLHRLKPDTVPAPC | |
| | CVPASYNPMVLIQKTDTGVSLQTYD | |
| | DLLAKDCHCI | |
| 17 | HGEGTFTSDVSSYLEEQAAKEFIAW | 49 |
| | LVKGGGGGGAGGGGAGGGGAGGGGA | |
| | DAHKSEVAHRFKDLGEENFKALVLI | |
| | AFAQYLQQSPFEDHVKLVNEVTEFA | |
| | KTCVADESAENCDKSLHTLFGDKLC | |
| | TVATLRETYGEMADCCAKQEPERNE | |
| | CFLQHKDDNPNLPRLVRPEVDVMCT | |
| | AFHDNEETFLKKYLYEIARRHPYFY | |
| | APELLFFAKRYKAAFTECCQAADKA | |
| | ACLLPKLDELRDEGKASSAKQRLKC | |
| | ASLQKFGERAFKAWAVARLSQRFPK | |
| | AEFAEVSKLVTDLTKVHTECCHGDL | |
| | LECADDRADLAKYICENQDSISSKL | |
| | KECCEKPLLEKSHCIAEVENDEMPA | |
| | DLPSLAADFVESKDVCKNYAEAKDV | |
| | FLGMFLYEYARRHPDYSVVLLLRLA | |
| | KTYETTLEKCCAAADPHECYAKVFD | |
| | EFKPLVEEPQNLIKQNCELFEQLGE | |
| | YKFQNALLVRYTKKVPQVSTPTLVE | |
| | VSRNLGKVGSKCCKHPEAKRMPCAE | |
| | DYLSVVLNQLCVLHEKTPVSDRVTK | |
| | CCTESLVNRRPCFSALEVDETYVPK | |
| | EFNAETFTFHADICTLSEKERQIKK | |
| | QTALVELVKHKPKATKEQLKAVMDD | |
| | FAAFVEKCCKADDKETCFAEEGKKL | |
| | VAASQAALGLGAGGGGAGGGGAGGG | |
| | GAGGGGAGGGGAGGGGAGGGGAGGG | |
| | GADHCPLGPGRCCRLHTVRASLEDL | |
| | GWADWVLSPREVQVTMCIGACPSQF | |
| | RAANMHAQIKTSLHRLKPDTVPAPC | |
| | CVPASYNPMVLIQKTDTGVSLQTYD | |
| | DLLAKDCHCI | |
| 18 | HGEGTFTSDVSSYLEEQAAKEFIAW | 50 |
| | LVKGGGGGGAGGGGAGGGGAGGGGA | |
| | GGGGADAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLGAGGGGAGGG | |
| | GAGGGGAGGGGAGGGGAGGGGAGGG | |
| | GAGGGGADHCPLGPGRCCRLHTVRA | |
| | SLEDLGWADWVLSPREVQVTMCIGA | |
| | CPSQFRAANMHAQIKTSLHRLKPDT | |
| | VPAPCCVPASYNPMVLIQKTDTGVS | |
| | LQTYDDLLAKDCHCI | |
| 19 | HGEGTFTSDVSSYLEEQAAKEFIAW | 51 |
| | LVKGGGAGGGGAGGGGAGGGGAGGG | |
| | GAGGGGAGGGGAGGGGAGGGGADAH | |
| | KSEVAHRFKDLGEENFKALVLIAFA | |
| | QYLQQSPFEDHVKLVNEVTEFAKTC | |
| | VADESAENCDKSLHTLFGDKLCTVA | |
| | TLRETYGEMADCCAKQEPERNECFL | |
| | QHKDDNPNLPRLVRPEVDVMCTAFH | |
| | DNEETFLKKYLYEIARRHPYFYAPE | |
| | LLFFAKRYKAAFTECCQAADKAACL | |
| | LPKLDELRDEGKASSAKQRLKCASL | |
| | QKFGERAFKAWAVARLSQRFPKAEF | |
| | AEVSKLVTDLTKVHTECCHGDLLEC | |
| | ADDRADLAKYICENQDSISSKLKEC | |
| | CEKPLLEKSHCIAEVENDEMPADLP | |
| | SLAADFVESKDVCKNYAEAKDVFLG | |
| | MFLYEYARRHPDYSVVLLLRLAKTY | |
| | ETTLEKCCAAADPHECYAKVFDEFK | |
| | PLVEEPQNLIKQNCELFEQLGEYKF | |
| | QNALLVRYTKKVPQVSTPTLVEVSR | |
| | NLGKVGSKCCKHPEAKRMPCAEDYL | |
| | SVVLNQLCVLHEKTPVSDRVTKCCT | |
| | ESLVNRRPCFSALEVDETYVPKEFN | |
| | AETFTFHADICTLSEKERQIKKQTA | |
| | LVELVKHKPKATKEQLKAVMDDFAA | |
| | FVEKCCKADDKETCFAEEGKKLVAA | |
| | SQAALGLGSGGGGSGGGGSGGGGSG | |
| | GGGSGGGGSGGGGSGGGGSGGGGSD | |
| | HCPLGPGRCCRLHTVRASLEDLGWA | |
| | DWVLSPREVQVTMCIGACPSQFRAA | |
| | NMHAQIKTSLHRLKPDTVPAPCCVP | |
| | ASYNPMVLIQKTDTGVSLQTYDDLL | |
| | AKDCHCI | |
| 20 | HGEGTFTSDLSKQMEEEAVRLFIEW | 52 |
| | LKNGGPSSGAPPPSGAGGGGAGGGG | |
| | ADAHKSEVAHRFKDLGEENFKALVL | |
| | IAFAQYLQQSPFEDHVKLVNEVTEF | |
| | AKTCVADESAENCDKSLHTLFGDKL | |
| | CTVATLRETYGEMADCCAKQEPERN | |
| | ECFLQHKDDNPNLPRLVRPEVDVMC | |
| | TAFHDNEETFLKKYLYEIARRHPYF | |
| | YAPELLFFAKRYKAAFTECCQAADK | |
| | AACLLPKLDELRDEGKASSAKQRLK | |
| | CASLQKFGERAFKAWAVARLSQRFP | |
| | KAEFAEVSKLVTDLTKVHTECCHGD | |
| | LLECADDRADLAKYICENQDSISSK | |
| | LKECCEKPLLEKSHCIAEVENDEMP | |
| | ADLPSLAADFVESKDVCKNYAEAKD | |
| | VFLGMFLYEYARRHPDYSVVLLLRL | |
| | AKTYETTLEKCCAAADPHECYAKVF | |
| | DEFKPLVEEPQNLIKQNCELFEQLG | |
| | EYKFQNALLVRYTKKVPQVSTPTLV | |
| | EVSRNLGKVGSKCCKHPEAKRMPCA | |
| | EDYLSVVLNQLCVLHEKTPVSDRVT | |
| | KCCTESLVNRRPCFSALEVDETYVP | |
| | KEFNAETFTFHADICTLSEKERQIK | |
| | KQTALVELVKHKPKATKEQLKAVMD | |
| | DFAAFVEKCCKADDKETCFAEEGKK | |
| | LVAASQAALGLGSGGGGSGGGGSGG | |
| | GGSGGGGSGGGGSGGGGSGGGGSGG | |
| | GGSDHCPLGPGRCCRLHTVRASLED | |
| | LGWADWVLSPREVQVTMCIGACPSQ | |
| | FRAANMHAQIKTSLHRLKPDTVPAP | |
| | CCVPASYNPMVLIQKTDTGVSLQTY | |
| | DDLLAKDCHCI | |
| 21 | HGEGTFTSDVSSYLEEQAAKEFIAW | 53 |
| | LVKGGGGGGAGGGGAGGGGAGGGGA | |
| | GGGGAGGGGADAHKSEVAHRFKDLG | |
| | EENFKALVLIAFAQYLQQSPFEDHV | |
| | KLVNEVTEFAKTCVADESAENCDKS | |
| | LHTLFGDKLCTVATLRETYGEMADC | |
| | CAKQEPERNECFLQHKDDNPNLPRL | |
| | VRPEVDVMCTAFHDNEETFLKKYLY | |
| | EIARRHPYFYAPELLFFAKRYKAAF | |
| | TECCQAADKAACLLPKLDELRDEGK | |
| | ASSAKQRLKCASLQKFGERAFKAWA | |
| | VARLSQRFPKAEFAEVSKLVTDLTK | |
| | VHTECCHGDLLECADDRADLAKYIC | |
| | ENQDSISSKLKECCEKPLLEKSHCI | |
| | AEVENDEMPADLPSLAADFVESKDV | |
| | CKNYAEAKDVFLGMFLYEYARRHPD | |
| | YSVVLLLRLAKTYETTLEKCCAAAD | |
| | PHECYAKVFDEFKPLVEEPQNLIKQ | |
| | NCELFEQLGEYKFQNALLVRYTKKV | |
| | PQVSTPTLVEVSRNLGKVGSKCCKH | |
| | PEAKRMPCAEDYLSVVLNQLCVLHE | |
| | KTPVSDRVTKCCTESLVNRRPCFSA | |
| | LEVDETYVPKEFNAETFTFHADICT | |
| | LSEKERQIKKQTALVELVKHKPKAT | |
| | KEQLKAVMDDFAAFVEKCCKADDKE | |
| | TCFAEEGKKLVAASQAALGLGSGGG | |
| | GSGGGGSGGGGSGGGGSGGGGSGGG | |
| | GSGGGGSGGGGSDHCPLGPGRCCRL | |
| | HTVRASLEDLGWADWVLSPREVQVT | |
| | MCIGACPSQFRAANMHAQIKTSLHR | |
| | LKPDTVPAPCCVPASYNPMVLIQKT | |
| | DTGVSLQTYDDLLAKDCHCI | |
| 22 | HGEGTFTSDVSSYLEEQAAKEFIAW | 54 |
| | LVKGGGGGGAGGGGAGGGGAGGGGA | |
| | GGGGADAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLGSGGGGSGGG | |
| | GSGGGGSGGGGSGGGGSGGGGSGGG | |
| | GSGGGGSDHCPLGPGRCCRLHTVRA | |
| | SLEDLGWADWVLSPREVQVTMCIGA | |
| | CPSQFRAANMHAQIKTSLHRLKPDT | |
| | VPAPCCVPASYNPMVLIQKTDTGVS | |
| | LQTYDDLLAKDCHCI | |
| 23 | HGEGTFTSDVSSYLEEQAAKEFIAW | 55 |
| | LVKGGGGGGSGGGGSGGGGSGGGGS | |
| | GGGGSGGGGSDAHKSEVAHRFKDLG | |
| | EENFKALVLIAFAQYLQQSPFEDHV | |
| | KLVNEVTEFAKTCVADESAENCDKS | |
| | LHTLFGDKLCTVATLRETYGEMADC | |
| | CAKQEPERNECFLQHKDDNPNLPRL | |
| | VRPEVDVMCTAFHDNEETFLKKYLY | |
| | EIARRHPYFYAPELLFFAKRYKAAF | |
| | TECCQAADKAACLLPKLDELRDEGK | |
| | ASSAKQRLKCASLQKFGERAFKAWA | |
| | VARLSQRFPKAEFAEVSKLVTDLTK | |
| | VHTECCHGDLLECADDRADLAKYIC | |
| | ENQDSISSKLKECCEKPLLEKSHCI | |
| | AEVENDEMPADLPSLAADFVESKDV | |
| | CKNYAEAKDVFLGMFLYEYARRHPD | |
| | YSVVLLLRLAKTYETTLEKCCAAAD | |
| | PHECYAKVFDEFKPLVEEPQNLIKQ | |
| | NCELFEQLGEYKFQNALLVRYTKKV | |
| | PQVSTPTLVEVSRNLGKVGSKCCKH | |
| | PEAKRMPCAEDYLSVVLNQLCVLHE | |
| | KTPVSDRVTKCCTESLVNRRPCFSA | |
| | LEVDETYVPKEFNAETFTFHADICT | |
| | LSEKERQIKKQTALVELVKHKPKAT | |
| | KEQLKAVMDDFAAFVEKCCKADDKE | |
| | TCFAEEGKKLVAASQAALGLGSGGG | |
| | GSGGGGSGGGGSGGGGSGGGGSGGG | |
| | GSGGGGSGGGGSDHCPLGPGRCCRL | |
| | HTVRASLEDLGWADWVLSPREVQVT | |
| | MCIGACPSQFRAANMHAQIKTSLHR | |
| | LKPDTVPAPCCVPASYNPMVLIQKT | |
| | DTGVSLQTYDDLLAKDCHCI | |
| 24 | HGEGTFTSDVSSYLEEQAAKEFIAW | 56 |
| | LVKGGGGGGSGGGGSGGGGSGGGGS | |
| | GGGGSDAHKSEVAHRFKDLGEENFK | |
| | ALVLIAFAQYLQQSPFEDHVKLVNE | |
| | VTEFAKTCVADESAENCDKSLHTLF | |
| | GDKLCTVATLRETYGEMADCCAKQE | |
| | PERNECFLQHKDDNPNLPRLVRPEV | |
| | DVMCTAFHDNEETFLKKYLYEIARR | |
| | HPYFYAPELLFFAKRYKAAFTECCQ | |
| | AADKAACLLPKLDELRDEGKASSAK | |
| | QRLKCASLQKFGERAFKAWAVARLS | |
| | QRFPKAEFAEVSKLVTDLTKVHTEC | |
| | CHGDLLECADDRADLAKYICENQDS | |
| | ISSKLKECCEKPLLEKSHCIAEVEN | |
| | DEMPADLPSLAADFVESKDVCKNYA | |
| | EAKDVFLGMFLYEYARRHPDYSVVL | |
| | LLRLAKTYETTLEKCCAAADPHECY | |
| | AKVFDEFKPLVEEPQNLIKQNCELF | |
| | EQLGEYKFQNALLVRYTKKVPQVST | |
| | PTLVEVSRNLGKVGSKCCKHPEAKR | |
| | MPCAEDYLSVVLNQLCVLHEKTPVS | |
| | DRVTKCCTESLVNRRPCFSALEVDE | |
| | TYVPKEFNAETFTFHADICTLSEKE | |
| | RQIKKQTALVELVKHKPKATKEQLK | |
| | AVMDDFAAFVEKCCKADDKETCFAE | |
| | EGKKLVAASQAALGLGSGGGGSGGG | |
| | GSGGGGSGGGGSGGGGSGGGGSGGG | |
| | GSGGGGSDHCPLGPGRCCRLHTVRA | |
| | SLEDLGWADWVLSPREVQVTMCIGA | |
| | CPSQFRAANMHAQIKTSLHRLKPDT | |
| | VPAPCCVPASYNPMVLIQKTDTGVS | |
| | LQTYDDLLAKDCHCI | |
| 25 | HGEGTFTSDVSSYLEEQAAKEFIAW | 57 |
| | LVKGGASAPAPAPAPAPAPAPAPAP | |
| | APGSDAHKSEVAHRFKDLGEENFKA | |
| | LVLIAFAQYLQQSPFEDHVKLVNEV | |
| | TEFAKTCVADESAENCDKSLHTLFG | |
| | DKLCTVATLRETYGEMADCCAKQEP | |
| | ERNECFLQHKDDNPNLPRLVRPEVD | |
| | VMCTAFHDNEETFLKKYLYEIARRH | |
| | PYFYAPELLFFAKRYKAAFTECCQA | |
| | ADKAACLLPKLDELRDEGKASSAKQ | |
| | RLKCASLQKFGERAFKAWAVARLSQ | |
| | RFPKAEFAEVSKLVTDLTKVHTECC | |
| | HGDLLECADDRADLAKYICENQDSI | |
| | SSKLKECCEKPLLEKSHCIAEVEND | |
| | EMPADLPSLAADFVESKDVCKNYAE | |
| | AKDVFLGMFLYEYARRHPDYSVVLL | |
| | LRLAKTYETTLEKCCAAADPHECYA | |
| | KVFDEFKPLVEEPQNLIKQNCELFE | |
| | QLGEYKFQNALLVRYTKKVPQVSTP | |
| | TLVEVSRNLGKVGSKCCKHPEAKRM | |
| | PCAEDYLSVVLNQLCVLHEKTPVSD | |
| | RVTKCCTESLVNRRPCFSALEVDET | |
| | YVPKEFNAETFTFHADICTLSEKER | |
| | QIKKQTALVELVKHKPKATKEQLKA | |
| | VMDDFAAFVEKCCKADDKETCFAEE | |
| | GKKLVAASQAALGLGSGGGGSGGGG | |
| | SGGGGSGGGGSGGGGSGGGGSGGGG | |
| | SGGGGSDHCPLGPGRCCRLHTVRAS | |
| | LEDLGWADWVLSPREVQVTMCIGAC | |
| | PSQFRAANMHAQIKTSLHRLKPDTV | |
| | PAPCCVPASYNPMVLIQKTDTGVSL | |
| | QTYDDLLAKDCHCI | |
| 26 | HGEGTFTSDVSSYLEEQAAKEFIAW | 58 |
| | LVKGGASGGGGSGGGGSGGGGSGGG | |
| | GSGGGGSGGGGSGGGGSGGGGSDAH | |
| | KSEVAHRFKDLGEENFKALVLIAFA | |
| | QYLQQSPFEDHVKLVNEVTEFAKTC | |
| | VADESAENCDKSLHTLFGDKLCTVA | |
| | TLRETYGEMADCCAKQEPLLFFAKR | |
| | YKAAFTECCQAADKAACLLPKLDEL | |
| | RDEGKASSAKQRLKCASLQKFGERA | |
| | FKAWAVARLSQRFPKAEFAEVSKLV | |
| | TDLTKVHTECCHGDLLECADDRADL | |
| | AKYICENQDSISSKLKECCEKPLLE | |
| | KSHCIAEVENDEMPADLPSLAADFV | |
| | ESKDVCKNYAEAKDVFLGMFLYEYA | |
| | RRHPDYSVVLLLRLAKTYETTLEKC | |
| | CAAADPHECYAKVFDEFKPLVEEPQ | |
| | NLIKQNCELFEQLGEYKFQNALLVR | |
| | YTKKVPQVSTPTLVEVSRNLGKVGS | |
| | KCCKHPEAKRMPCAEDYLSVVLNQL | |
| | CVLHEKTPVSDRVTKCCTESLVNRR | |
| | PCFSALEVDETYVPKEFNAETFTFH | |
| | ADICTLSEKERQIKKQTALVELVKH | |
| | KPKATKEQLKAVMDDFAAFVEKCCK | |
| | ADDKETCFAEEGKKLVAASQAALGL | |
| | GSGGGGSGGGGSGGGGSGGGGSGGG | |
| | GSGGGGSGGGGSGGGGSDHCPLGPG | |
| | RCCRLHTVRASLEDLGWADWVLSPR | |
| | EVQVTMCIGACPSQFRAANMHAQIK | |
| | TSLHRLKPDTVPAPCCVPASYNPMV | |
| | LIQKTDTGVSLQTYDDLLAKDCHCI | |
| 27 | HGEGTFTSDLSKQMEEEAVRLFIEW | 59 |
| | LKNGGPSSGAPPPSASGGGGSGGGG | |
| | SGGGGSGGGGSGGGGSGGGGSGGGG | |
| | SGGGGSDAHKSEVAHRFKDLGEENF | |
| | KALVLIAFAQYLQQSPFEDHVKLVN | |
| | EVTEFAKTCVADESAENCDKSLHTL | |
| | FGDKLCTVATLRETYGEMADCCAKQ | |
| | EPERNECFLQHKDDNPNLPRLVRPE | |
| | VDVMCTAFHDNEETFLKKYLYEIAR | |
| | RHPYFYAPELLFFAKRYKAAFTECC | |
| | QAADKAACLLPKLDELRDEGKASSA | |
| | KQRLKCASLQKFGERAFKAWAVARL | |
| | SQRFPKAEFAEVSKLVTDLTKVHTE | |
| | CCHGDLLECADDRADLAKYICENQD | |
| | SISSKLKECCEKPLLEKSHCIAEVE | |
| | NDEMPADLPSLAADFVESKDVCKNY | |
| | AEAKDVFLGMFLYEYARRHPDYSVV | |
| | LLLRLAKTYETTLEKCCAAADPHEC | |
| | YAKVFDEFKPLVEEPQNLIKQNCEL | |
| | FEQLGEYKFQNALLVRYTKKVPQVS | |
| | TPTLVEVSRNLGKVGSKCCKHPEAK | |
| | RMPCAEDYLSVVLNQLCVLHEKTPV | |
| | SDRVTKCCTESLVNRRPCFSALEVD | |
| | ETYVPKEFNAETFTFHADICTLSEK | |
| | ERQIKKQTALVELVKHKPKATKEQL | |
| | KAVMDDFAAFVEKCCKADDKETCFA | |
| | EEGKKLVAASQAALGLGSGGGGSGG | |
| | GGSGGGGSGGGGSGGGGSGGGGSGG | |
| | GGSGGGGSDHCPLGPGRCCRLHTVR | |
| | ASLEDLGWADWVLSPREVQVTMCIG | |
| | ACPSQFRAANMHAQIKTSLHRLKPD | |
| | TVPAPCCVPASYNPMVLIQKTDTGV | |
| | SLQTYDDLLAKDCHCI | |
| 28 | HGEGTFTSDLSKQMEEEAVRLFIEW | 60 |
| | LKNGGPSSGAPPPSASAPAPAPAPA | |
| | PAPAPAPAPAPGSDAHKSEVAHRFK | |
| | DLGEENFKALVLIAFAQYLQQSPFE | |
| | DHVKLVNEVTEFAKTCVADESAENC | |
| | DKSLHTLFGDKLCTVATLRETYGEM | |
| | ADCCAKQEPERNECFLQHKDDNPNL | |
| | PRLVRPEVDVMCTAFHDNEETFLKK | |
| | YLYEIARRHPYFYAPELLFFAKRYK | |
| | AAFTECCQAADKAACLLPKLDELRD | |
| | EGKASSAKQRLKCASLQKFGERAFK | |
| | AWAVARLSQRFPKAEFAEVSKLVTD | |
| | LTKVHTECCHGDLLECADDRADLAK | |
| | YICENQDSISSKLKECCEKPLLEKS | |
| | HCIAEVENDEMPADLPSLAADFVES | |
| | KDVCKNYAEAKDVFLGMFLYEYARR | |
| | HPDYSVVLLLRLAKTYETTLEKCCA | |
| | AADPHECYAKVFDEFKPLVEEPQNL | |
| | IKQNCELFEQLGEYKFQNALLVRYT | |
| | KKVPQVSTPTLVEVSRNLGKVGSKC | |
| | CKHPEAKRMPCAEDYLSVVLNQLCV | |
| | LHEKTPVSDRVTKCCTESLVNRRPC | |
| | FSALEVDETYVPKEFNAETFTFHAD | |
| | ICTLSEKERQIKKQTALVELVKHKP | |
| | KATKEQLKAVMDDFAAFVEKCCKAD | |
| | DKETCFAEEGKKLVAASQAALGLGS | |
| | GGGGSGGGGSGGGGSGGGGSGGGGS | |
| | GGGGSGGGGSGGGGSDHCPLGPGRC | |
| | CRLHTVRASLEDLGWADWVLSPREV | |
| | QVTMCIGACPSQFRAANMHAQIKTS | |
| | LHRLKPDTVPAPCCVPASYNPMVLI | |
| | QKTDTGVSLQTYDDLLAKDCHCI | |
| 29 | HSEGTFTSDVSSYLEGQAAKEFIEW | 61 |
| | LVKGRASAPAPAPAPAPAPAPAPAP | |
| | APGSDAHKSEVAHRFKDLGEENFKA | |
| | LVLIAFAQYLQQSPFEDHVKLVNEV | |
| | TEFAKTCVADESAENCDKSLHTLFG | |
| | DKLCTVATLRETYGEMADCCAKQEP | |
| | ERNECFLQHKDDNPNLPRLVRPEVD | |
| | VMCTAFHDNEETFLKKYLYEIARRH | |
| | PYFYAPELLFFAKRYKAAFTECCQA | |
| | ADKAACLLPKLDELRDEGKASSAKQ | |
| | RLKCASLQKFGERAFKAWAVARLSQ | |
| | RFPKAEFAEVSKLVTDLTKVHTECC | |
| | HGDLLECADDRADLAKYICENQDSI | |
| | SSKLKECCEKPLLEKSHCIAEVEND | |
| | EMPADLPSLAADFVESKDVCKNYAE | |
| | AKDVFLGMFLYEYARRHPDYSVVLL | |
| | LRLAKTYETTLEKCCAAADPHECYA | |
| | KVFDEFKPLVEEPQNLIKQNCELFE | |
| | QLGEYKFQNALLVRYTKKVPQVSTP | |
| | TLVEVSRNLGKVGSKCCKHPEAKRM | |
| | PCAEDYLSVVLNQLCVLHEKTPVSD | |
| | RVTKCCTESLVNRRPCFSALEVDET | |
| | YVPKEFNAETFTFHADICTLSEKER | |
| | QIKKQTALVELVKHKPKATKEQLKA | |
| | VMDDFAAFVEKCCKADDKETCFAEE | |
| | GKKLVAASQAALGLGSGGGGSGGGG | |
| | SGGGGSGGGGSGGGGSGGGGSGGGG | |
| | SGGGGSDHCPLGPGRCCRLHTVRAS | |
| | LEDLGWADWVLSPREVQVTMCIGAC | |
| | PSQFRAANMHAQIKTSLHRLKPDTV | |
| | PAPCCVPASYNPMVLIQKTDTGVSL | |
| | QTYDDLLAKDCHCI | |
| 30 | HSEGTFTSDVSSYLEGQAAKEFIEW | 62 |
| | LVKGRASGGGGSGGGGSGGGGSGGG | |
| | GSGGGGSGGGGSGGGGSGGGGSDAH | |
| | KSEVAHRFKDLGEENFKALVLIAFA | |
| | QYLQQSPFEDHVKLVNEVTEFAKTC | |
| | VADESAENCDKSLHTLFGDKLCTVA | |
| | TLRETYGEMADCCAKQEPERNECFL | |
| | QHKDDNPNLPRLVRPEVDVMCTAFH | |
| | DNEETFLKKYLYEIARRHPYFYAPE | |
| | LLFFAKRYKAAFTECCQAADKAACL | |
| | LPKLDELRDEGKASSAKQRLKCASL | |
| | QKFGERAFKAWAVARLSQRFPKAEF | |
| | AEVSKLVTDLTKVHTECCHGDLLEC | |
| | ADDRADLAKYICENQDSISSKLKEC | |
| | CEKPLLEKSHCIAEVENDEMPADLP | |
| | SLAADFVESKDVCKNYAEAKDVFLG | |
| | MFLYEYARRHPDYSVVLLLRLAKTY | |
| | ETTLEKCCAAADPHECYAKVFDEFK | |
| | PLVEEPQNLIKQNCELFEQLGEYKF | |
| | QNALLVRYTKKVPQVSTPTLVEVSR | |
| | NLGKVGSKCCKHPEAKRMPCAEDYL | |
| | SVVLNQLCVLHEKTPVSDRVTKCCT | |
| | ESLVNRRPCFSALEVDETYVPKEFN | |
| | AETFTFHADICTLSEKERQIKKQTA | |
| | LVELVKHKPKATKEQLKAVMDDFAA | |
| | FVEKCCKADDKETCFAEEGKKLVAA | |
| | SQAALGLGSGGGGSGGGGSGGGGSG | |
| | GGGSGGGGSGGGGSGGGGSGGGGSD | |
| | HCPLGPGRCCRLHTVRASLEDLGWA | |
| | DWVLSPREVQVTMCIGACPSQFRAA | |
| | NMHAQIKTSLHRLKPDTVPAPCCVP | |
| | ASYNPMVLIQKTDTGVSLQTYDDLL | |
| | AKDCHCI | |
| 31 | HGEGTFTSDVSSYLEEQAAKEFIAW | 63 |
| | LVKGGGGGGSGGGGSGGGGSDAHKS | |
| | EVAHRFKDLGEENFKALVLIAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAKRYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCIAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALV | |
| | ELVKHKPKATKEQLKAVMDDFAAFV | |
| | EKCCKADDKETCFAEEGKKLVAASQ | |
| | AALGLGSGGGGSGGGGSGGGGSGGG | |
| | GSGGGGSGGGGSGGGGSGGGGSDHC | |
| | PLGPGRCCRLHTVRASLEDLGWADW | |
| | VLSPREVQVTMCIGACPSQFRAANM | |
| | HAQIKTSLHRLKPDTVPAPCCVPAS | |
| | YNPMVLIQKTDTGVSLQTYDDLLAK | |
| | DCHCI | |
| 32 | HGEGTFTSDLSKQMEEEAVRLFIEW | 64 |
| | LKNGGPSSGAPPPSGGGGSGGGGSG | |
| | GGGSDAHKSEVAHRFKDLGEENFKA | |
| | LVLIAFAQYLQQSPFEDHVKLVNEV | |
| | TEFAKTCVADESAENCDKSLHTLFG | |
| | DKLCTVATLRETYGEMADCCAKQEP | |
| | ERNECFLQHKDDNPNLPRLVRPEVD | |
| | VMCTAFHDNEETFLKKYLYEIARRH | |
| | PYFYAPELLFFAKRYKAAFTECCQA | |
| | ADKAACLLPKLDELRDEGKASSAKQ | |
| | RLKCASLQKFGERAFKAWAVARLSQ | |
| | RFPKAEFAEVSKLVTDLTKVHTECC | |
| | HGDLLECADDRADLAKYICENQDSI | |
| | SSKLKECCEKPLLEKSHCIAEVEND | |
| | EMPADLPSLAADFVESKDVCKNYAE | |
| | AKDVFLGMFLYEYARRHPDYSVVLL | |
| | LRLAKTYETTLEKCCAAADPHECYA | |
| | KVFDEFKPLVEEPQNLIKQNCELFE | |
| | QLGEYKFQNALLVRYTKKVPQVSTP | |
| | TLVEVSRNLGKVGSKCCKHPEAKRM | |
| | PCAEDYLSVVLNQLCVLHEKTPVSD | |
| | RVTKCCTESLVNRRPCFSALEVDET | |
| | YVPKEFNAETFTFHADICTLSEKER | |
| | QIKKQTALVELVKHKPKATKEQLKA | |
| | VMDDFAAFVEKCCKADDKETCFAEE | |
| | GKKLVAASQAALGLGSGGGGSGGGG | |
| | SGGGGSGGGGSGGGGSGGGGSGGGG | |
| | SGGGGSDHCPLGPGRCCRLHTVRAS | |
| | LEDLGWADWVLSPREVQVTMCIGAC | |
| | PSQFRAANMHAQIKTSLHRLKPDTV | |
| | PAPCCVPASYNPMVLIQKTDTGVSL | |
| | QTYDDLLAKDCHCI | |
| 33 | HSEGTFTSDVSSYLEGQAAKEFIEW | 65 |
| | LVKGRASGGGGSGGGGSDAHKSEVA | |
| | HRFKDLGEENFKALVLIAFAQYLQQ | |
| | SPFEDHVKLVNEVTEFAKTCVADES | |
| | AENCDKSLHTLFGDKLCTVATLRET | |
| | YGEMADCCAKQEPERNECFLQHKDD | |
| | NPNLPRLVRPEVDVMCTAFHDNEET | |
| | FLKKYLYEIARRHPYFYAPELLFFA | |
| | KRYKAAFTECCQAADKAACLLPKLD | |
| | ELRDEGKASSAKQRLKCASLQKFGE | |
| | RAFKAWAVARLSQRFPKAEFAEVSK | |
| | LVTDLTKVHTECCHGDLLECADDRA | |
| | DLAKYICENQDSISSKLKECCEKPL | |
| | LEKSHCIAEVENDEMPADLPSLAAD | |
| | FVESKDVCKNYAEAKDVFLGMFLYE | |
| | YARRHPDYSVVLLLRLAKTYETTLE | |
| | KCCAAADPHECYAKVFDEFKPLVEE | |
| | PQNLIKQNCELFEQLGEYKFQNALL | |
| | VRYTKKVPQVSTPTLVEVSRNLGKV | |
| | GSKCCKHPEAKRMPCAEDYLSVVLN | |
| | QLCVLHEKTPVSDRVTKCCTESLVN | |
| | RRPCFSALEVDETYVPKEFNAETFT | |
| | FHADICTLSEKERQIKKQTALVELV | |
| | KHKPKATKEQLKAVMDDFAAFVEKC | |
| | CKADDKETCFAEEGKKLVAASQAAL | |
| | GLGSGGGGSGGGGSGGGGSGGGGSG | |
| | GGGSGGGGSGGGGSGGGGSDHCPLG | |
| | PGRCCRLHTVRASLEDLGWADWVLS | |
| | PREVQVTMCIGACPSQFRAANMHAQ | |
| | IKTSLHRLKPDTVPAPCCVPASYNP | |
| | MVLIQKTDTGVSLQTYDDLLAKDCH | |
| | CI | |
| 34 | HSEGTFTSDVSSYLEGQAAKEFIEW | 66 |
| | LVKGRASAPAPAPAPAPGSDAHKSE | |
| | VAHRFKDLGEENFKALVLIAFAQYL | |
| | QQSPFEDHVKLVNEVTEFAKTCVAD | |
| | ESAENCDKSLHTLFGDKLCTVATLR | |
| | ETYGEMADCCAKQEPERNECFLQHK | |
| | DDNPNLPRLVRPEVDVMCTAFHDNE | |
| | ETFLKKYLYEIARRHPYFYAPELLF | |
| | FAKRYKAAFTECCQAADKAACLLPK | |
| | LDELRDEGKASSAKQRLKCASLQKF | |
| | GERAFKAWAVARLSQRFPKAEFAEV | |
| | SKLVTDLTKVHTECCHGDLLECADD | |
| | RADLAKYICENQDSISSKLKECCEK | |
| | PLLEKSHCIAEVENDEMPADLPSLA | |
| | ADFVESKDVCKNYAEAKDVFLGMFL | |
| | YEYARRHPDYSVVLLLRLAKTYETT | |
| | LEKCCAAADPHECYAKVFDEFKPLV | |
| | EEPQNLIKQNCELFEQLGEYKFQNA | |
| | LLVRYTKKVPQVSTPTLVEVSRNLG | |
| | KVGSKCCKHPEAKRMPCAEDYLSVV | |
| | LNQLCVLHEKTPVSDRVTKCCTESL | |
| | VNRRPCFSALEVDETYVPKEFNAET | |
| | FTFHADICTLSEKERQIKKQTALVE | |
| | LVKHKPKATKEQLKAVMDDFAAFVE | |
| | KCCKADDKETCFAEEGKKLVAASQA | |
| | ALGLGSGGGGSGGGGSGGGGSGGGG | |
| | SGGGGSGGGGSGGGGSGGGGSDHCP | |
| | LGPGRCCRLHTVRASLEDLGWADWV | |
| | LSPREVQVTMCIGACPSQFRAANMH | |
| | AQIKTSLHRLKPDTVPAPCCVPASY | |
| | NPMVLIQKTDTGVSLQTYDDLLAKD | |
| | CHCI | |
| 35 | HGEGTFTSDLSKQMEEEAVRLFIEW | 67 |
| | LKNGGPSSGAPPPSASGGGGSGGGG | |
| | SDAHKSEVAHRFKDLGEENFKALVL | |
| | IAFAQYLQQSPFEDHVKLVNEVTEF | |
| | AKTCVADESAENCDKSLHTLFGDKL | |
| | CTVATLRETYGEMADCCAKQEPERN | |
| | ECFLQHKDDNPNLPRLVRPEVDVMC | |
| | TAFHDNEETFLKKYLYEIARRHPYF | |
| | YAPELLFFAKRYKAAFTECCQAADK | |
| | AACLLPKLDELRDEGKASSAKQRLK | |
| | CASLQKFGERAFKAWAVARLSQRFP | |
| | KAEFAEVSKLVTDLTKVHTECCHGD | |
| | LLECADDRADLAKYICENQDSISSK | |
| | LKECCEKPLLEKSHCIAEVENDEMP | |
| | ADLPSLAADFVESKDVCKNYAEAKD | |
| | VFLGMFLYEYARRHPDYSVVLLLRL | |
| | AKTYETTLEKCCAAADPHECYAKVF | |
| | DEFKPLVEEPQNLIKQNCELFEQLG | |
| | EYKFQNALLVRYTKKVPQVSTPTLV | |
| | EVSRNLGKVGSKCCKHPEAKRMPCA | |
| | EDYLSVVLNQLCVLHEKTPVSDRVT | |
| | KCCTESLVNRRPCFSALEVDETYVP | |
| | KEFNAETFTFHADICTLSEKERQIK | |
| | KQTALVELVKHKPKATKEQLKAVMD | |
| | DFAAFVEKCCKADDKETCFAEEGKK | |
| | LVAASQAALGLGSGGGGSGGGGSGG | |
| | GGSGGGGSGGGGSGGGGSGGGGSGG | |
| | GGSDHCPLGPGRCCRLHTVRASLED | |
| | LGWADWVLSPREVQVTMCIGACPSQ | |
| | FRAANMHAQIKTSLHRLKPDTVPAP | |
| | CCVPASYNPMVLIQKTDTGVSLQTY | |
| | DDLLAKDCHCI | |
| 36 | HGEGTFTSDLSKQMEEEAVRLFIEW | 68 |
| | LKNGGPSSGAPPPSASAPAPAPAPA | |
| | PGSDAHKSEVAHRFKDLGEENFKAL | |
| | VLIAFAQYLQQSPFEDHVKLVNEVT | |
| | EFAKTCVADESAENCDKSLHTLFGD | |
| | KLCTVATLRETYGEMADCCAKQEPE | |
| | RNECFLQHKDDNPNLPRLVRPEVDV | |
| | MCTAFHDNEETFLKKYLYEIARRHP | |
| | YFYAPELLFFAKRYKAAFTECCQAA | |
| | DKAACLLPKLDELRDEGKASSAKQR | |
| | LKCASLQKFGERAFKAWAVARLSQR | |
| | FPKAEFAEVSKLVTDLTKVHTECCH | |
| | GDLLECADDRADLAKYICENQDSIS | |
| | SKLKECCEKPLLEKSHCIAEVENDE | |
| | MPADLPSLAADFVESKDVCKNYAEA | |
| | KDVFLGMFLYEYARRHPDYSVVLLL | |
| | RLAKTYETTLEKCCAAADPHECYAK | |
| | VFDEFKPLVEEPQNLIKQNCELFEQ | |
| | LGEYKFQNALLVRYTKKVPQVSTPT | |
| | LVEVSRNLGKVGSKCCKHPEAKRMP | |
| | CAEDYLSVVLNQLCVLHEKTPVSDR | |
| | VTKCCTESLVNRRPCFSALEVDETY | |
| | VPKEFNAETFTFHADICTLSEKERQ | |
| | IKKQTALVELVKHKPKATKEQLKAV | |
| | MDDFAAFVEKCCKADDKETCFAEEG | |
| | KKLVAASQAALGLGSGGGGSGGGGS | |
| | GGGGSGGGGSGGGGSGGGGSGGGGS | |
| | GGGGSDHCPLGPGRCCRLHTVRASL | |
| | EDLGWADWVLSPREVQVTMCIGACP | |
| | SQFRAANMHAQIKTSLHRLKPDTVP | |
| | APCCVPASYNPMVLIQKTDTGVSLQ | |
| | TYDDLLAKDCHCI | |
| 37 | HGEGTFTSDVSSYLEEQAAKEFIAW | 69 |
| | LVKGGASGGGGSGGGGSDAHKSEVA | |
| | HRFKDLGEENFKALVLIAFAQYLQQ | |
| | SPFEDHVKLVNEVTEFAKTCVADES | |
| | AENCDKSLHTLFGDKLCTVATLRET | |
| | YGEMADCCAKQEPERNECFLQHKDD | |
| | NPNLPRLVRPEVDVMCTAFHDNEET | |
| | FLKKYLYEIARRHPYFYAPELLFFA | |
| | KRYKAAFTECCQAADKAACLLPKLD | |
| | ELRDEGKASSAKQRLKCASLQKFGE | |
| | RAFKAWAVARLSQRFPKAEFAEVSK | |
| | LVTDLTKVHTECCHGDLLECADDRA | |
| | DLAKYICENQDSISSKLKECCEKPL | |
| | LEKSHCIAEVENDEMPADLPSLAAD | |
| | FVESKDVCKNYAEAKDVFLGMFLYE | |
| | YARRHPDYSVVLLLRLAKTYETTLE | |
| | KCCAAADPHECYAKVFDEFKPLVEE | |
| | PQNLIKQNCELFEQLGEYKFQNALL | |
| | VRYTKKVPQVSTPTLVEVSRNLGKV | |
| | GSKCCKHPEAKRMPCAEDYLSVVLN | |
| | QLCVLHEKTPVSDRVTKCCTESLVN | |
| | RRPCFSALEVDETYVPKEFNAETFT | |
| | FHADICTLSEKERQIKKQTALVELV | |
| | KHKPKATKEQLKAVMDDFAAFVEKC | |
| | CKADDKETCFAEEGKKLVAASQAAL | |
| | GLGSGGGGSGGGGSGGGGSGGGGSG | |
| | GGGSGGGGSGGGGSGGGGSDHCPLG | |
| | PGRCCRLHTVRASLEDLGWADWVLS | |
| | PREVQVTMCIGACPSQFRAANMHAQ | |
| | IKTSLHRLKPDTVPAPCCVPASYNP | |
| | MVLIQKTDTGVSLQTYDDLLAKDCH | |
| | CI | |
| 38 | HGEGTFTSDVSSYLEEQAAKEFIAW | 70 |
| | LVKGGASAPAPAPAPAPGSDAHKSE | |
| | VAHRFKDLGEENFKALVLIAFAQYL | |
| | QQSPFEDHVKLVNEVTEFAKTCVAD | |
| | ESAENCDKSLHTLFGDKLCTVATLR | |
| | ETYGEMADCCAKQEPERNECFLQHK | |
| | DDNPNLPRLVRPEVDVMCTAFHDNE | |
| | ETFLKKYLYEIARRHPYFYAPELLF | |
| | FAKRYKAAFTECCQAADKAACLLPK | |
| | LDELRDEGKASSAKQRLKCASLQKF | |
| | GERAFKAWAVARLSQRFPKAEFAEV | |
| | SKLVTDLTKVHTECCHGDLLECADD | |
| | RADLAKYICENQDSISSKLKECCEK | |
| | PLLEKSHCIAEVENDEMPADLPSLA | |
| | ADFVESKDVCKNYAEAKDVFLGMFL | |
| | YEYARRHPDYSVVLLLRLAKTYETT | |
| | LEKCCAAADPHECYAKVFDEFKPLV | |
| | EEPQNLIKQNCELFEQLGEYKFQNA | |
| | LLVRYTKKVPQVSTPTLVEVSRNLG | |
| | KVGSKCCKHPEAKRMPCAEDYLSVV | |
| | LNQLCVLHEKTPVSDRVTKCCTESL | |
| | VNRRPCFSALEVDETYVPKEFNAET | |
| | FTFHADICTLSEKERQIKKQTALVE | |
| | LVKHKPKATKEQLKAVMDDFAAFVE | |
| | KCCKADDKETCFAEEGKKLVAASQA | |
| | ALGLGSGGGGSGGGGSGGGGSGGGG | |
| | SGGGGSGGGGSGGGGSGGGGSDHCP | |
| | LGPGRCCRLHTVRASLEDLGWADWV | |
| | LSPREVQVTMCIGACPSQFRAANMH | |
| | AQIKTSLHRLKPDTVPAPCCVPASY | |
| | NPMVLIQKTDTGVSLQTYDDLLAKD | |
| | CHCI | |
| 39 | HSEGTFTSDVSSYLEGQAAKEFIEW | 71 |
| | LVKGRGGGGSGGGGSGGGGSDAHKS | |
| | EVAHRFKDLGEENFKALVLIAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAKRYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCIAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALV | |
| | ELVKHKPKATKEQLKAVMDDFAAFV | |
| | EKCCKADDKETCFAEEGKKLVAASQ | |
| | AALGLGSGGGGSGGGGSGGGGSGGG | |
| | GSGGGGSGGGGSGGGGSGGGGSDHC | |
| | PLGPGRCCRLHTVRASLEDLGWADW | |
| | VLSPREVQVTMCIGACPSQFRAANM | |
| | HAQIKTSLHRLKPDTVPAPCCVPAS | |
| | YNPMVLIQKTDTGVSLQTYDDLLAK | |
| | DCHCI | |
| 40 | HGEGTFTSDVSSYLEEQAAKEFIAW | 12 |
| | LVKGGGGGGSGGGGSGGGGSDAHKS | |
| | EVAHRFKDLGEETFKALVLVAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAARYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCLAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALA | |
| | ELVKHKPKATKEQLKTVMDDFAAFV | |
| | EKCCKADDKETCFAEEGKKLVAASQ | |
| | AALGLGSGGGGSGGGGSGGGGSGGG | |
| | GSGGGGSGGGGSGGGGSGGGGSDHC | |
| | PLGPGRCCRLHTVRASLEDLGWADW | |
| | VLSPREVQVTMCIGACPSQFRAANM | |
| | HAQIKTSLHRLKPDTVPAPCCVPAS | |
| | YNPMVLIQKTDTGVSLQTYDDLLAK | |
| | DCHCI | |
| 41 | HGEGTFTSDVSSYLEEQAAKEFIAW | 73 |
| | LVKGGGGGGSGGGGSGGGGSDAHKS | |
| | EVAHRFKDLGEETFKALVLVAFAQY | |
| | LQQSPFEDHVKLVNEVTEFAKTCVA | |
| | DESAENCDKSLHTLFGDKLCTVATL | |
| | RETYGEMADCCAKQEPERNECFLQH | |
| | KDDNPNLPRLVRPEVDVMCTAFHDN | |
| | EETFLKKYLYEIARRHPYFYAPELL | |
| | FFAARYKAAFTECCQAADKAACLLP | |
| | KLDELRDEGKASSAKQRLKCASLQK | |
| | FGERAFKAWAVARLSQRFPKAEFAE | |
| | VSKLVTDLTKVHTECCHGDLLECAD | |
| | DRADLAKYICENQDSISSKLKECCE | |
| | KPLLEKSHCLAEVENDEMPADLPSL | |
| | AADFVESKDVCKNYAEAKDVFLGMF | |
| | LYEYARRHPDYSVVLLLRLAKTYET | |
| | TLEKCCAAADPHECYAKVFDEFKPL | |
| | VEEPQNLIKQNCELFEQLGEYKFQN | |
| | ALLVRYTKKVPQVSTPTLVEVSRNL | |
| | GKVGSKCCKHPEAKRMPCAEDYLSV | |
| | VLNQLCVLHEKTPVSDRVTKCCTES | |
| | LVNRRPCFSALEVDETYVPKEFNAE | |
| | TFTFHADICTLSEKERQIKKQTALA | |
| | ELVKHKPKATKEQLKTVMDDFAAFV | |
| | EKCCKADDKETCFAEEGKKLVAASQ | |
| | AALGLGSGGGGSGGGGSGGGGSGGG | |
| | GSGGGGSGGGGSGGGGSGGGGSDHC | |
| | PLGPGRCCRLHTVRASLEDLGWADW | |
| | VLSPREVQVTMCIGACPSQFRAANM | |
| | HAQIKTSLHRLKPDTVPAPCCVPAS | |
| | YNPMVLRQKTDTGVSLQTYDDLLAK | |
| | DCHCI | |
| 42 | EGTFTSDVSSYLEEQAAKEFIAWLV | 74 |
| | KGGGGGGSGGGGSGGGGSDAHKSEV | |
| | AHRFKDLGEETFKALVLVAFAQYLQ | |
| | QSPFEDHVKLVNEVTEFAKTCVADE | |
| | SAENCDKSLHTLFGDKLCTVATLRE | |
| | TYGEMADCCAKQEPERNECFLQHKD | |
| | DNPNLPRLVRPEVDVMCTAFHDNEE | |
| | TFLKKYLYEIARRHPYFYAPELLFF | |
| | AARYKAAFTECCQAADKAACLLPKL | |
| | DELRDEGKASSAKQRLKCASLQKFG | |
| | ERAFKAWAVARLSQRFPKAEFAEVS | |
| | KLVTDLTKVHTECCHGDLLECADDR | |
| | ADLAKYICENQDSISSKLKECCEKP | |
| | LLEKSHCLAEVENDEMPADLPSLAA | |
| | DFVESKDVCKNYAEAKDVFLGMFLY | |
| | EYARRHPDYSVVLLLRLAKTYETTL | |
| | EKCCAAADPHECYAKVFDEFKPLVE | |
| | EPQNLIKQNCELFEQLGEYKFQNAL | |
| | LVRYTKKVPQVSTPTLVEVSRNLGK | |
| | VGSKCCKHPEAKRMPCAEDYLSVVL | |
| | NQLCVLHEKTPVSDRVTKCCTESLV | |
| | NRRPCFSALEVDETYVPKEFNAETF | |
| | TFHADICTLSEKERQIKKQTALAEL | |
| | VKHKPKATKEQLKTVMDDFAAFVEK | |
| | CCKADDKETCFAEEGKKLVAASQAA | |
| | LGLGSGGGGSGGGGSGGGGSGGGGS | |
| | GGGGSGGGGSGGGGSGGGGSDHCPL | |
| | GPGRCCRLHTVRASLEDLGWADWVL | |
| | SPREVQVTMCIGACPSQFRAANMHA | |
| | QIKTSLHRLKPDTVPAPCCVPASYN | |
| | PMVLIQKTDTGVSLQTYDDLLAKDC | |
| | HCI | |

### GLP1-GDF15 Fusion Polynucleotides and Vectors

In another general aspect, the invention relates to an isolated nucleic acid encoding the GLP1-GDF15 fusion proteins of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding the fusion proteins of the invention can be altered without changing the amino acid sequences of the proteins.

In another general aspect, the invention relates to a vector comprising an isolated nucleic acid encoding a fusion protein of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of a fusion protein in the cell. Conventional cloning techniques or artificial gene synthesis can be used to generate a recombinant expression vector according to embodiments of the invention.

In another general aspect, the invention relates to a host cell comprising an isolated nucleic acid encoding a fusion protein of the invention. Any host cell known to those skilled in the art in view of the present disclosure can be used for recombinant expression of fusion proteins of the invention. In some embodiments, the host cells are E. coli TG1 or BL21 cells, CHO-DG44 or CHO-K1 cells or HEK293 cells. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a fusion protein of the invention, comprising culturing a cell comprising a nucleic acid encoding the fusion protein under conditions to produce a fusion protein of the invention, and recovering the fusion protein from the cell or cell culture (e.g., from the supernatant). Expressed fusion proteins can be harvested from the cells and purified according to conventional techniques known in the art and as described herein.

### Pharmaceutical Compositions

In another general aspect, the invention relates to a pharmaceutical composition, comprising the GLP1-GDF15 fusion proteins and/or GLP1-GDF15 fusion polynucleotides of the invention and a pharmaceutically acceptable carrier. The term “pharmaceutical composition” as used herein means a product comprising a GLP1-GDF15 fusion proteins and/or GLP1-GDF15 fusion polynucleotides of the invention together with a pharmaceutically acceptable carrier. GLP1-GDF15 fusion proteins and/or GLP1-GDF15 fusion polynucleotides of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a peptide pharmaceutical composition can be used in the invention.

Pharmaceutically acceptable acidic/anionic salts for use in the invention include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl) aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

In some embodiments of the invention, pharmaceutical formulations are provided comprising the GLP1-GDF15 fusion proteins and/or GLP1-GDF15 fusion polynucleotides of the invention in an amount from about 0.001 mg/ml to about 100 mg/ml, from about 0.01 mg/ml to about 50 mg/ml, or from about 0.1 mg/ml to about 25 mg/ml. The pharmaceutical formulation can have a pH from about 3.0 to about 10, for example from about 3 to about 7, or from about 5 to about 9. The formulation can further comprise at least one ingredient selected from the group consisting of a buffer system, preservative(s), tonicity agent(s), chelating agent(s), stabilizer(s) and surfactant(s).

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of the embodiment include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. Pharmaceutical compositions comprising each isotonic agent listed in this paragraph constitute alternative embodiments of the invention. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

The pharmaceutical composition of the invention can comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid can be present either in its free base form or in its salt form. Any stereoisomer (i.e., L, D, or a mixture thereof) of the amino acid base can be present. The amino acid base can be present individually or in the combination with other amino acid bases, in a concentration from about 0.01 mg/ml to about 50 mg/ml, for example from about 0.1 mg/ml to about 20 mg/ml. Pharmaceutical compositions comprising each one of these specific amino acid bases constitute alternative embodiments of the invention.

The pharmaceutically-acceptable salts of the GLP1-GDF15 fusion proteins and/or GLP1-GDF15 fusion polynucleotides of the invention include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups can be quaternized with, for example, alkyl halides.

The pharmaceutical compositions of the invention can be administered by any means that accomplish their intended purpose. Examples include administration by parenteral, subcutaneous, intravenous, intramuscular, intraperitoneal, transdermal, buccal or ocular routes. Administration can be by the oral route. Suitable formulations for parenteral administration include aqueous solutions of the active conjugates in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the GLP1-GDF15 fusion proteins and/or GLP1-GDF15 fusion polynucleotides of the present invention. Additionally, the present invention includes pharmaceutical compositions made by mixing one or more pharmaceutically acceptable carriers with any of the GLP1-GDF15 fusion proteins and/or GLP1-GDF15 polynucleotides of the present invention.

Furthermore, the GLP1-GDF15 fusion proteins and/or GLP1-GDF15 fusion polynucleotides of the present invention can have one or more polymorph or amorphous crystalline forms and as such are intended to be included in the scope of the invention. In addition, the GLP1-GDF15 fusion proteins and/or GLP1-GDF15 fusion polynucleotides can form solvates, for example with water (i.e., hydrates) or common organic solvents. As used herein, the term “solvate” means a physical association of the GLP1-GDF15 fusion proteins and/or GLP1-GDF15 fusion polynucleotides of the present invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the GLP1-GDF15 fusion proteins and/or GLP1-GDF15 fusion polynucleotides of the present invention. Thus, in the methods of treatment of the present invention, the term “administering” shall encompass the means for treating, ameliorating or preventing a syndrome, disorder or disease described herein with the GLP1-GDF15 fusion proteins and/or GLP1-GDF15 fusion polynucleotides of the present invention or a polymorph or solvate thereof, which would obviously be included within the scope of the invention albeit not specifically disclosed.

In another embodiment, the invention relates to the GLP1-GDF15 fusion proteins and/or GLP1-GDF15 fusion polynucleotides of the invention for use as a medicament.

The present invention includes within its scope prodrugs of the GLP1-GDF15 fusion proteins and/or GLP1-GDF15 fusion polynucleotides of this invention. In general, such prodrugs will be functional derivatives of the GLP1-GDF15 fusion proteins and/or GLP1-GDF15 fusion polynucleotides, which are readily convertible in vivo into the required GLP1-GDF15 fusion proteins and/or GLP1-GDF15 fusion polynucleotides. Thus, in the methods of treatment of the present invention, the term “administering” shall encompass the treatment of the various disorders described with the GLP1-GDF15 fusion proteins and/or GLP1-GDF15 fusion polynucleotides specifically disclosed or with a GLP1-GDF15 fusion proteins and/or GLP1-GDF15 fusion polynucleotides, which may not be specifically disclosed, but which converts to the specified GLP1-GDF15 fusion proteins and/or GLP1-GDF15 fusion polynucleotides in vivo after administration to the patient. Conventional procedures for the selection and preparation of suitable prodrug derivatives are described, for example, in “Design of Prodrugs”, Ed. H. Bundgaard, Elsevier, 1985.

During any of the processes for preparation of the GLP1-GDF15 fusion proteins and/or GLP1-GDF15 fusion polynucleotides of the present invention, it can be necessary and/or desirable to protect sensitive or reactive groups on any of the molecules concerned. This can be achieved by means of conventional protecting groups, such as those described in Protective Groups in Organic Chemistry, ed. J.F.W. McOmie, Plenum Press, 1973; and T.W. Greene & P.G.M. Wuts, Protective Groups in Organic Synthesis, John Wiley & Sons, 1991, each of which is herein incorporated by reference in its entirety for all purposes. The protecting groups can be removed at a convenient subsequent stage using methods known from the art.

### Methods of Use

The present invention is directed to a method for preventing, treating or ameliorating a GDF15 receptor (GDF15R, GFRAL) mediated syndrome and/or a GLP1 receptor mediated syndrome, disorder or disease in a subject in need thereof comprising administering to the subject in need thereof an effective amount of a GLP1-GDF15 fusion protein, GLP1-GDF15 fusion polynucleotide, and/or pharmaceutical composition of the invention.

The present invention also provides a method for preventing, treating, delaying the onset of, or ameliorating a disorder, disease, or condition or any one or more symptoms of said disorder, disease, or condition in a subject in need thereof, comprising administering to the subject in need thereof an effective amount of a GLP1-GDF15 fusion protein, GLP1-GDF15 fusion polynucleotide, and/or pharmaceutical composition of the invention.

According to particular embodiments, the disease disorder, or condition is selected from the group consisting of obesity, type I or II diabetes, metabolic syndrome (i.e., Syndrome X), insulin resistance, impaired glucose tolerance (e.g., glucose intolerance), hyperglycemia, hyperinsulinemia, hypertriglyceridemia, hypoglycemia due to congenital hyperinsulinism (CHI), dyslipidemia, atherosclerosis, diabetic nephropathy, and other cardiovascular risk factors such as hypertension and cardiovascular risk factors related to unmanaged cholesterol and/or lipid levels, osteoporosis, inflammation, non-alcoholic fatty liver disease (NAFLD), non-alcoholic steatohepatitis (NASH), renal disease, eczema, sleep apnea, osteoarthritis, polycystic ovarian syndrome, chronic kidney syndrome, depression, and/or cancer.

According to particular embodiments, a therapeutically effective amount refers to the amount of therapy which is sufficient to achieve one, two, three, four, or more of the following effects: (i) reduce or ameliorate the severity of the disease, disorder, or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; (xii) enhance or improve the prophylactic or therapeutic effect(s) of another therapy; and/or (xiii) improve quality of life of a subject with the disease, disorder or condition to be treated.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

As used herein, the terms “treat,” “treating,” and “treatment” are all intended to refer to an amelioration or reversal of at least one measurable physical parameter related the disease, disorder, or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In one embodiment, the invention provides a method for preventing, treating, delaying the onset of, or ameliorating obesity, or any one or more symptoms of obesity in a subject in need thereof, the method comprising administering to the subject in need thereof an effective amount of a GLP1-GDF15 fusion protein, GLP1-GDF15 fusion polynucleotide, and/or pharmaceutical composition of the invention. In some embodiments, the body weight of a subject is reduced, for example, by between about 0.01% to about 0.1%, between about 0.1% to about 0.5%, between about 0.5% to about 1%, between about 1% to about 5%, between about 2% to about 3%, between about 5% to about 10%, between about 10% to about 15%, between about 15% to about 20%, between about 20% to about 25%, between about 25% to about 30%, between about 30% to about 35%, between about 35% to about 40%, between about 40% to about 45%, or between about 45% to about 50%, relative to the body weight of a subject prior to administration of any of the GLP1-GDF15 fusion proteins, GLP1-GDF15 fusion polynucleotides, pharmaceutical compositions, forms, or medicaments of the invention described herein, or compared to control subjects not receiving any of the GLP1-GDF15 fusion proteins, GLP1-GDF15 fusion polynucleotides, compositions, forms, medicaments, or combinations of the invention described herein.

In some embodiments, the reduction in body weight is maintained for about 1 week, for about 2 weeks, for about 3 weeks, for about 1 month, for about 2 months, for about 3 months, for about 4 months, for about 5 months, for about 6 months, for about 7 months, for about 8 months, for about 9 months, for about 10 months, for about 11 months, for about 1 year, for about 1.5 years, for about 2 years, for about 2.5 years, for about 3 years, for about 3.5 years, for about 4 years, for about 4.5 years, for about 5 years, for about 6 years, for about 7 years, for about 8 years, for about 9 years, for about 10 years, for about 15 years, or for about 20 years, for example.

The present invention provides a method of preventing, treating, delaying the onset of, or ameliorating a syndrome, disorder or disease, or any one or more symptoms of said syndrome, disorder, or disease in a subject in need thereof, wherein said syndrome, disorder or disease is selected from the group consisting of obesity, type I or type II diabetes, metabolic syndrome (i.e., Syndrome X), insulin resistance, impaired glucose tolerance (e.g., glucose intolerance), hyperglycemia, hyperinsulinemia, hypertriglyceridemia, hypoglycemia due to congenital hyperinsulinism (CHI), dyslipidemia, atherosclerosis, diabetic nephropathy, and other cardiovascular risk factors such as hypertension and cardiovascular risk factors related to unmanaged cholesterol and/or lipid levels, osteoporosis, inflammation, non-alcoholic fatty liver disease (NAFLD), non-alcoholic steatohepatitis (NASH), renal disease, and eczema, comprising administering to the subject in need thereof an effective amount of a GLP1-GDF15 fusion protein, GLP1-GDF15 fusion polynucleotide, and/or pharmaceutical composition of the invention.

As used herein, metabolic syndrome refers to a subject having any one or more of the following: high blood sugar (e.g., high fasting blood sugar), high blood pressure, abnormal cholesterol levels (e.g., low HDL levels), abnormal triglyceride levels (e.g., high triglycerides), a large waistline (i.e., waist circumference), increased fat in the abdominal area, insulin resistance, glucose intolerance, elevated C-reactive protein levels (i.e., a proinflammatory state), and increased plasma plasminogen activator inhibitor-1 and fibrinogen levels (i.e., a prothrombotic state).

The present invention provides a method of reducing food intake in a subject in need thereof, the method comprising administering to the subject in need thereof an effective amount of a GLP1-GDF15 fusion protein, GLP1-GDF15 fusion polynucleotide, and/or pharmaceutical composition of the invention. In some embodiments, food intake of a subject is reduced, for example, by between about 0.01% to about 0.1%, between about 0.1% to about 0.5%, between about 0.5% to about 1%, between about 1% to about 5%, between about 2% to about 3%, between about 5% to about 10%, between about 10% to about 15%, between about 15% to about 20%, between about 20% to about 25%, between about 25% to about 30%, between about 30% to about 35%, between about 35% to about 40%, between about 40% to about 45%, or between about 45% to about 50%, relative to food intake of a subject prior to administration of any of the GLP1-GDF15 fusion proteins, GLP1-GDF15 fusion polynucleotides, compositions, forms, medicaments, or combinations of the invention described herein, or compared to control subjects not receiving any of the GLP1-GDF15 fusion proteins, GLP1-GDF15 fusion polynucleotides, compositions, forms, medicaments, or combinations of the invention described herein.

In some embodiments, the reduction in food intake is maintained for about 1 week, for about 2 weeks, for about 3 weeks, for about 1 month, for about 2 months, for about 3 months, for about 4 months, for about 5 months, for about 6 months, for about 7 months, for about 8 months, for about 9 months, for about 10 months, for about 11 months, for about 1 year, for about 1.5 years, for about 2 years, for about 2.5 years, for about 3 years, for about 3.5 years, for about 4 years, for about 4.5 years, for about 5 years, for about 6 years, for about 7 years, for about 8 years, for about 9 years, for about 10 years, for about 15 years, or for about 20 years, for example.

The present invention provides a method of reducing glycated hemoglobin (A1C) in a subject in need thereof, the method comprising administering to the subject in need thereof an effective amount of a GLP1-GDF15 fusion protein, GLP1-GDF15 fusion polynucleotide, and/or pharmaceutical composition of the invention. In some embodiments, A1C of a subject is reduced, for example, by between about 0.001% and about 0.01%, between about 0.01% and about 0.1%, between about 0.1% and about 0.2%, between about 0.2% and about 0.3%, between about 0.3% and about 0.4%, between about 0.4% and about 0.5%, between about 0.5% and about 1%, between about 1% and about 1.5%, between about 1.5% and about 2%, between about 2% and about 2.5%, between about 2.5% and about 3%, between about 3% and about 4%, between about 4% and about 5%, between about 5% and about 6%, between about 6% and about 7%, between about 7% and about 8%, between about 8% and about 9%, or between about 9% and about 10% relative to the A1C of a subject prior to administration of any of the GLP1-GDF15 fusion proteins, GLP1-GDF15 fusion polynucleotides, compositions, forms, medicaments, or combinations of the invention described herein, or compared to control subjects not receiving any of the GLP1-GDF15 fusion proteins, GLP1-GDF15 fusion polynucleotides, compositions, forms, medicaments, or combinations of the invention described herein.

In other embodiments, methods are provided for reducing fasting blood glucose levels in a subject in need thereof, the methods comprising administering to the subject in need thereof an effective amount of a GLP1-GDF15 fusion protein, GLP1-GDF15 fusion polynucleotide, and/or pharmaceutical composition of the invention. Fasting blood glucose levels may be reduced to less than about 140 to about 150 mg/dL, less than about 140 to about 130 mg/dL, less than about 130 to about 120 mg/dL, less than about 120 to about 110 mg/dL, less than about 110 to about 100 mg/dL, less than about 100 to about 90 mg/dL, or less than about 90 to about 80 mg/dL, relative to the fasting blood glucose levels of a subject prior to administration of any of the GLP1-GDF15 fusion proteins, GLP1-GDF15 fusion polynucleotides, compositions, forms, medicaments, or combinations of the invention described herein, or compared to control subjects not receiving any of the GLP1-GDF15 fusion proteins, GLP1-GDF15 fusion polynucleotides, compositions, forms, medicaments, or combinations of the invention described herein.

The present invention provides a method of modulating GLP1 receptor activity and GDF15 receptor activity in a subject in need thereof, the method comprising administering to the subject in need thereof an effective amount of a GLP1-GDF15 fusion protein, GLP1-GDF15 fusion polynucleotide, and/or pharmaceutical composition of the invention. As used herein, “modulating” refers to increasing or decreasing receptor activity.

In some embodiments, an effective amount of a GLP1-GDF15 fusion protein and/or GLP1-GDF15 fusion polynucleotide of the invention or a form, composition or medicament thereof is administered to a subject in need thereof once daily, twice daily, three times daily, four times daily, five times daily, six times daily, seven times daily, or eight times daily. In other embodiments, an effective amount of a GLP1-GDF15 fusion protein and/or GLP1-GDF15 fusion polynucleotide of the invention or a form, composition or medicament thereof is administered to a subject in need thereof once every other day, once per week, twice per week, three times per week, four times per week, five times per week, six times per week, two times per month, three times per month, or four times per month.

Another embodiment of the invention comprises a method of preventing, treating, delaying the onset of, or ameliorating a disease, disorder or syndrome, or one or more symptoms of any of said diseases, disorders, or syndromes in a subject in need thereof, the method comprising administering to the subject in need thereof an effective amount of a GLP1-GDF15 fusion protein, GLP1-GDF15 fusion polynucleotide, and/or pharmaceutical composition of the invention in a combination therapy. In certain embodiments, the combination therapy is a second therapeutic agent. In certain embodiments, the combination therapy is a surgical therapy.

As used herein, the term “in combination,” in the context of the administration of two or more therapies to a subject, refers to the use of more than one therapy.

As used herein, combination therapy refers to administering to a subject in need thereof one or more additional therapeutic agents, or one or more surgical therapies, concurrently with an effective amount of a GLP1-GDF15 fusion protein and/or GLP1-GDF15 fusion polynucleotide of the invention or a form, composition or medicament thereof. In some embodiments, the one or more additional therapeutic agents or surgical therapies can be administered on the same day as an effective amount of a GLP1-GDF15 fusion protein and/or GLP1-GDF15 fusion polynucleotide of the invention, and in other embodiments, the one or more additional therapeutic agents or surgical therapies may be administered in the same week or the same month as an effective amount of a GLP1-GDF15 fusion protein and/or GLP1-GDF15 fusion polynucleotide of the invention.

The present invention also contemplates preventing, treating, delaying the onset of, or ameliorating any of the diseases, disorders, syndromes, or symptoms described herein in a subject in need thereof with a combination therapy that comprises administering to the subject in need thereof an effective amount of a GLP1-GDF15 fusion protein, GLP1-GDF15 fusion polynucleotide, and/or pharmaceutical composition of the invention, in combination with any one or more of the following therapeutic agents: a dipeptidyl peptidase-4 (DPP-4) inhibitor (e.g., sitagliptin, saxagliptin, linagliptin, alogliptin, etc.); a GLP1 receptor agonist (e.g., short-acting GLP1 receptor agonists such as exenatide and lixisenatide; intermediate-acting GLP1 receptor agonists such as liraglutide; long-acting GLP1 receptor agonists such as exenatide extended-release, albiglutide, dulaglutide); a sodium-glucose co-transporter-2 (SGLT-2) inhibitors (e.g., canaglifozin, dapaglifozin, empaglifozin, etc.); bile acid sequestrants (e.g., colesevelam, etc.); dopamine receptor agonists (e.g., bromocriptine quick-release); biguanides (e.g., metformin, etc.); insulin; oxyntomodulin; sulfonylureas (e.g., chlorpropamide, glimepiride, glipizide, glyburide, glibenclamide, glibornuride, glisoxepide, glyclopyramide, tolazamide, tolbutamide, acetohexamide, carbutamide, etc.); and thiazolidinediones (e.g; pioglitazone, rosiglitazone, lobeglitazone, ciglitazone, darglitazone, englitazone, netoglitazone, rivoglitazone, troglitazone, etc.). In some embodiments, the dose of the additional therapeutic agent(s) is reduced when given in combination with a GLP1-GDF15 fusion protein and/or GLP1-GDF15 fusion polynucleotide of the invention. In some embodiments, when used in combination with a GLP1-GDF15 fusion protein and/or GLP1-GDF15 fusion polynucleotide of the invention, the additional therapeutic agent(s) may be used in lower doses than when each is used singly.

The present invention contemplates preventing, treating, delaying the onset of, or ameliorating any of the diseases, disorders, syndromes, or symptoms described herein in a subject in need thereof, with a combination therapy that comprises administering to the subject in need thereof an effective amount of a GLP1-GDF15 fusion protein, GLP1-GDF15 fusion polynucleotide, and/or pharmaceutical composition of the invention in combination with a surgical therapy. In certain embodiments, the surgical therapy can be bariatric surgery (e.g., gastric bypass surgery, such as Roux-en-Y gastric bypass surgery; sleeve gastrectomy; adjustable gastric band surgery; biliopancreatic diversion with duodenal switch; intragastric balloon; gastric plication; and combinations thereof).

In embodiments in which the one or more additional therapeutic agents or surgical therapies is administered on the same day as an effective amount of a GLP1-GDF15 fusion protein and/or GLP1-GDF15 fusion polynucleotide of the invention, the GLP1-GDF15 fusion protein and/or GLP1-GDF15 fusion polynucleotide of the invention may be administered prior to, after, or simultaneously with the additional therapeutic agent or surgical therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### EMBODIMENTS

The invention provides also the following non-limiting embodiments.

Embodiment 1 is a glucagon-like peptide-1 (GLP1)/growth differentiation factor 15 (GDF15) fusion protein, wherein the GLP1-GDF15 fusion protein comprises a GLP1 peptide, a first linker peptide, a serum albumin protein, a second linker peptide, and a GDF15 protein.

Embodiment 2 is the GLP1-GDF15 fusion protein of embodiment 1, wherein GLP1 peptide comprises an amino acid sequence selected from the group consisting of SEQ ID NOs:1-4.

Embodiment 3 is the GLP1-GDF15 fusion protein of embodiment 1 or 2, wherein the first linker peptide comprises an amino acid sequence selected from the group consisting of SEQ ID NOs:5-25.

Embodiment 4 is the GLP1-GDF15 fusion protein of any one of embodiments 1-3, wherein the serum albumin protein comprises an amino acid sequence selected from SEQ ID NO:26 or SEQ ID NO:27.

Embodiment 5 is the GLP1-GDF15 fusion protein of any one of embodiments 1-4, wherein the second linker peptide comprises an amino acid sequence selected from the group consisting of SEQ ID NOs:28-30.

Embodiment 6 is the GLP1-GDF15 fusion protein of any one of embodiments 1-5, wherein the GDF15 protein comprises an amino acid sequence selected from SEQ ID NO:31 or SEQ ID NO:32.

Embodiment 7 is a GLP1-GDF15 fusion protein, wherein the GLP1-GDF15 fusion protein comprises an amino acid sequence selected from the group consisting of SEQ ID NOs:33-74 and 84.

Embodiment 8 is an isolated nucleic acid encoding the GLP1-GDF15 fusion protein of any one of embodiments 1-7.

Embodiment 9 is a vector comprising the isolated nucleic acid of embodiment 8.

Embodiment 10 is a host cell comprising the isolated nucleic acid of embodiment 8 or the vector of claim 9.

Embodiment 11 is a pharmaceutical composition comprising the GLP1-GDF15 fusion protein of any one of embodiments 1-7 and a pharmaceutically acceptable carrier.

Embodiment 12 is a method for treating or preventing obesity in a subject in need thereof, comprising administering to the subject in need thereof an effective amount of the pharmaceutical composition of embodiment 11.

Embodiment 13 is the method of embodiment 12, wherein administration of the effective amount of the pharmaceutical composition to the subject in need thereof results in a reduction in body weight of about 5% to about 10%, about 10% to about 15%, about 15% to about 20%, or about 20% to about 25% as compared to the body weight of the subject prior to administration of the pharmaceutical composition.

Embodiment 14 is a method for treating or preventing a disease or disorder in a subject in need thereof, wherein said disease or disorder is selected from the group consisting of obesity, type I or type II diabetes, metabolic syndrome, insulin resistance, impaired glucose tolerance, hyperglycemia, hyperinsulinemia, hypertriglyceridemia, hypoglycemia due to congenital hyperinsulinism (CHI), dyslipidemia, atherosclerosis, diabetic nephropathy, and other cardiovascular risk factors such as hypertension and cardiovascular risk factors related to unmanaged cholesterol and/or lipid levels, osteoporosis, inflammation, non-alcoholic fatty liver disease (NAFLD), non-alcoholic steatohepatitis (NASH), renal disease, and eczema, the method comprising administering to the subject in need thereof an effective amount of the pharmaceutical composition of embodiment 11.

Embodiment 15 is the method of embodiment 14, wherein said disease or disorder is obesity.

Embodiment 16 is the method of embodiment 14, wherein said disease or disorder is type I diabetes.

Embodiment 17 is the method of embodiment 14, wherein said disease or disorder is type II diabetes.

Embodiment 18 is the method of embodiment 14, wherein said disease or disorder is a metabolic syndrome.

Embodiment 19 is the method of embodiment 14, wherein said disease or disorder is a renal disease.

Embodiment 20 is the method of embodiment 14, wherein said disease or disorder is non-alcoholic steatohepatitis (NASH).

Embodiment 21 is the method of embodiment 14, wherein said disease or disorder is non-alcoholic fatty liver disease (NAFLD).

Embodiment 22 is a method of reducing food intake in a subject in need thereof, the method comprising administering to the subject in need thereof an effective amount of the pharmaceutical composition of embodiment 11.

Embodiment 23 is the method of embodiment 22, wherein administration of the effective amount of the pharmaceutical composition to the subject in need thereof results in a reduction in food intake of about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% as compared to the food intake of the subject prior to administration of the pharmaceutical composition.

Embodiment 24 is a method of modulating GLP1 receptor activity in a subject in need thereof, the method comprising administering to the subject in need thereof an effective amount of the pharmaceutical composition of embodiment 11.

Embodiment 25 is a method of modulating GDF15 receptor activity in a subject in need thereof, the method comprising administering to the subject in need thereof an effective amount of the pharmaceutical composition of embodiment 11.

Embodiment 26 is the method of any one of embodiments 11-25, wherein the pharmaceutical composition is administered via an injection.

Embodiment 27 is the method of embodiment 26, wherein the injection is delivered subcutaneously, intramuscularly, intraperitoneally, or intravenously.

Embodiment 28 is the method of any one of embodiments 11-27, wherein the pharmaceutical composition is administered in combination with a second therapeutic agent.

Embodiment 29 is the method of any one of embodiments 11-28, wherein the pharmaceutical composition is administered daily, weekly, or monthly to the subject in need thereof.

Embodiment 30 is the method of embodiment 29, wherein the pharmaceutical composition is administered once, twice, three, four, five, or six times per day.

Embodiment 31 is the method of embodiment 29, wherein the pharmaceutical composition is administered once, twice, three, four, five, or six times per week.

Embodiment 32 is the method of embodiment 29, wherein the pharmaceutical composition is administered once, twice, three, or four times per month.

Embodiment 33 is a kit comprising the GLP1-GDF15 fusion protein of any one of embodiments 1-7, the isolated nucleic acid of embodiment 8, and/or the vector of embodiment 9.

Embodiment 34 is the kit of embodiment 33, wherein the kit further comprises a device for injection.

Embodiment 35 is a method of producing a pharmaceutical composition comprising the GLP1-GDF15 fusion protein of any one of embodiments 1-7, comprising combining the GLP1-GDF15 fusion protein with a pharmaceutically acceptable carrier to obtain the pharmaceutical composition.

Embodiment 36 is a method of producing the GLP1-GDF15 fusion protein of any one of embodiments 1-7, comprising culturing a cell comprising a nucleic acid encoding the GLP1-GDF15 fusion protein under conditions to produce the GLP1-GDF15 fusion protein, and recovering the GLP1-GDF15 fusion protein from the cell or culture.

### EXAMPLES

### Example 1: Combination of GLP1 and GDF15 Agonists

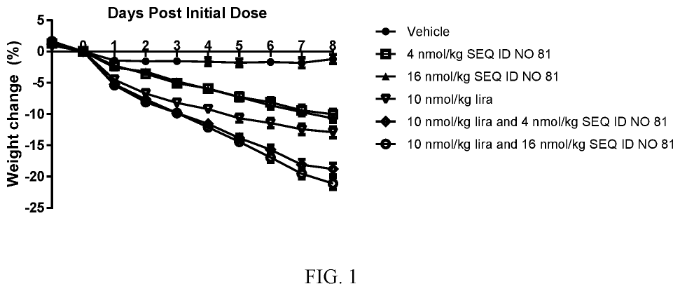

The potential additive weight loss effects of combining GLP1 and GDF15 agonists were tested in diet induced obese (DIO) mice. Liraglutide, a GLP1 agonist, was administered alone or in combination with an HSA-GDF15 (SEQ ID NO:81) molecule for 8 days. Liraglutide was subcutaneously administered daily while HSA-GDF15 was given every two days. Body weight loss was greater in the animals receiving both agonists compared to either single agent demonstrating the potential for additivity when combining these independent mechanisms (FIG. 1).

### Example 2: Design of the GLP1-GDF15 Fusion Proteins

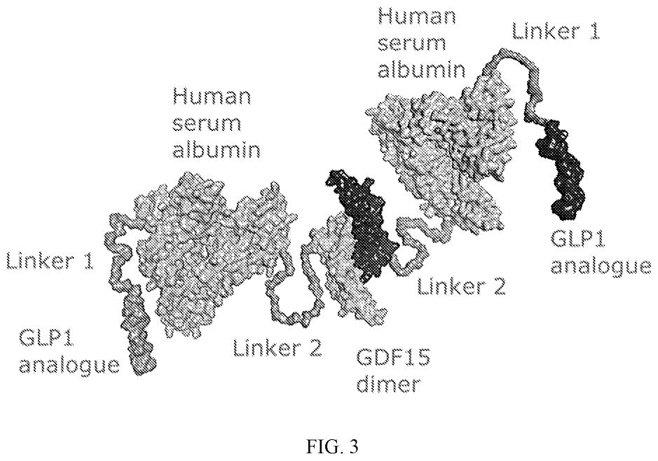

Recombinant fusion proteins comprised of a GLP1 peptide or GLP1 variant peptide, serum albumin protein, and GDF15 protein or GDF15 variant protein were designed as follows: GLP1 peptide or GLP1 variant peptides were connected via a first linker peptide at the C-terminus of the GLP1 peptide or GLP1 variant peptide to the N-terminus of human serum albumin. The human serum albumin is connected at the C-terminus to the N-terminus of the GDF15 protein or GDF15 protein variant via a second linker peptide. The design aims to leave the GDF15 dimerization interface unperturbed and allow for formation of the native inter-chain disulfide, resulting in a homodimer of approximately 170 KDa. The entire molecules were designed to be made recombinantly, needing only one gene (FIG. 3).

GLP1 peptides or GLP1 variant peptides include either human GLP1(7-36) peptide with mutations or Exendin 4(9-39) peptide, a venom peptide from Gila monster that agonizes the human GLP1 receptor. SEQ ID NO:1 contains (A8S, A30E) mutations (numbering for mutation positions refer to GLP1 peptide mutations in Table 1, not from the starting of fusion protein) and SEQ ID NO:2 contains (A8G, G22E, R36G) mutations from human GLP1 peptide (UniProtKB-P01275 98-127). SEQ ID NO:3 contains the Exendin 4 peptide (UniProtKB-P26349 48-86).

Native human serum albumin (UniProtKB-P02768 25-609) contains 35 cysteine (Cys, C) residues which form 17 intramolecular disulfide bonds, leaving Cys-34 as the only free cysteine. This free Cys-34 has been shown to function as a free radical scavenger, by trapping multiple reactive oxygen species (ROS) and reactive nitrogen species (RNS) (Taverna et. al, Ann Intensive Care, 3:4 (2013)). This free Cys was therefore mutated to Ser to create SEQ ID NO:26 to minimize chemical reactivity and the risk of heterogeneity due to oxidation.

The N terminus of mature GDF15 (UniProtKB-Q99988 197-308) contains a proteolytic liability site (R198) and a deamidation liability site (N199). Therefore, the fusion proteins contain GDF15(201-308) (SEQ ID NO:31) with those liability sites deleted.

### Example 3: Expression and Purification Methods

GLP1-GDF15 fusion proteins, GLP1-first linker-serum albumin (e.g., HSA, GSA) proteins, serum albumin (e.g., HSA, GSA)-second linker-GDF15 proteins, and/or GDF15 proteins utilized in the examples above were expressed either in HEK Expi293F™ (ThermoFisher Scientific, Cat #A14527) or ExpiCHO-S™ (ThermoFisher Scientific, Cat #A29127; Waltham, MA). For expression in HEK Expi293F™, a plasmid encoding the GLP1-GDF15 fusion protein was transfected into cells by transient transfection following the manufacturer's recommendations. Briefly, Expi293F cells were maintained in suspension in Expi293™ expression medium (ThermoFisher Scientific) in a shaking incubator set at 37° C., 8% CO₂ and 125 RPM. The cells were passaged so that on the day of transfection, dilution down to 2.5×10⁶ cells per ml could be achieved, maintaining cell viability at 95% or better. Transient transfections were performed using the ExpiFectamine™ 293 transfection kit (ThermoFisher Scientific). For each ml of diluted cells to be transfected, one microgram of plasmid DNA was diluted into OptiMEM™ SFM complexation medium. ExpiFectamine™ 293 reagent was used at a 1:2.6 ratio (v/v, DNA:reagent) and also diluted into OptiMEM™ and allowed to incubate for 5 minutes at room temperature. The diluted DNA and transfection reagent were combined for twenty minutes, allowing DNA/lipid complex formation, and then added to the cells. After overnight incubation, Expi293™ feed and ExpiFectamine™ 293 enhancer were added to the cells. Cells were cultured with shaking at 37° C. for four days prior to harvesting the culture supernatants.

ExpiCHO-S™ cells were maintained in suspension in ExpiCHO™ expression medium (ThermoFisher Scientific) in a shaking incubator set at 37° C., 8% CO₂ and 125 RPM. The cells were passaged so that on the day of transfection, dilution down to 6.0×10⁶ cells per ml could be achieved, maintaining cell viability at 98% or better. Transient transfections were done using the ExpiFectamine™ CHO transfection kit (ThermoFisher Scientific). For each ml of diluted cells to be transfected, one microgram of plasmid DNA is diluted into OptiPRO™ SFM complexation medium. ExpiFectamine™ CHO reagent is used at a 1:3 ratio (v/v, DNA:reagent) and also diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancer were added to the cells. Cells were cultured with shaking at 32° C. for five days prior to harvesting the culture supernatants.

GLP1-GDF15 fusion proteins were purified from harvested culture supernatant either by single-step affinity capture or a two-step process using affinity capture followed by a preparative size exclusion chromatography (SEC) polishing step. Cell supernatants from transiently transfected ExpiCHO™ cells were loaded onto a pre-equilibrated (dPBS, pH 7.2) HSA CaptureSelect column (CaptureSelect Human Albumin Affinity Matrix from ThermoFisher Scientific) at an approximate capacity of 10 mg protein per ml of resin. After loading, unbound proteins and impurities were removed by washing the column with up to 12 column volumes (CV) of dPBS pH 7.2 followed by 3 CV of 1M NaCl in 50 mM Sodium phosphate, pH 7.4. The GLP1-GDF15 fusion protein which bound to the column was eluted with up to 10 CV of 0.1M Sodium Acetate, pH 3.5, into fraction tubes containing 10 percent (by volume) of 1M Tris (untitrated). Peak fractions were pooled and filtered over a 0.2 μm membrane, then either buffer exchanged into dPBS pH 7.2 or continued to SEC step at 4° C.

For SEC step, the protein from the capture step was concentrated to an appropriate volume before loading onto a 26/60 superdex 200 column (GE Healthcare; Little Chalfont, United Kingdom). Protein fractions eluted from SEC column with high purity (determined by SDS-PAGE) were pooled. The concentration of protein (from either method) was determined by the absorbance at 280 nm on a BioTek Synergy HTTM spectrophotometer. The quality of the purified proteins was assessed by SDS-PAGE and analytical size exclusion HPLC (SE-HPLC, Dionex HPLC system). Endotoxin levels were measured using a LAL assay (Pyrotell®-T, Associates of Cape Cod; East Falmouth, MA).

### Example 4: Dual Target Engagement and Additive Effects with Delivery of Both GLP1 and GDF15 Agonists on One Molecule

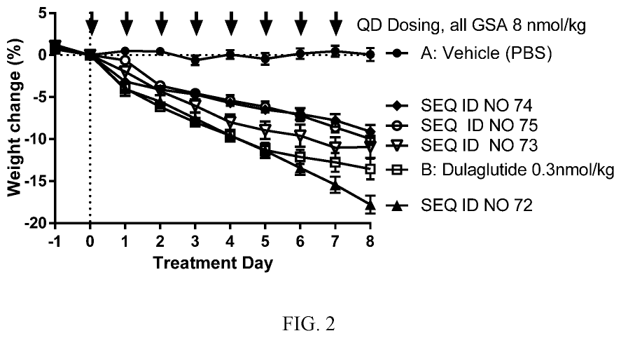

The potential to achieve additive effects through delivering both GLP1 and GDF15 agonists on one molecule was tested by assessing weight loss in DIO mice over eight days of treatment with fusions of the agonists with gorilla serum albumin (GSA). GSA was used as a surrogate for HSA in these tool molecules. Mice received subcutaneous administration of 8 nmol/kg of GLP1-GSA-GDF15 (SEQ ID NO:72), GLP1-GSA-GDF15(I89R) (SEQ ID NO:73, I89R mutation abolishes GDF15 activity), GLP1(9-36)-GSA-GDF15 (SEQ ID NO:74, or GSA-GDF15 (SEQ ID NO:75), the latter two both designed to represent degraded GLP1. Two additional groups of animals served as controls and references, one receiving vehicle alone or another that was administered the GLP1 agonist dulaglutide. Mice treated with GLP1-GSA-GDF15 (SEQ ID NO:72) had greater weight loss than those treated with GLP1(9-36)-GSA-GDF15 (SEQ ID NO:74), GLP1-GSA-GDF15(I89R) (SEQ ID NO:73), and GSA-GDF15 (SEQ ID NO:75), providing proof of concept that both GLP1R and GFRAL can be agonized with a single molecule resulting in additive efficacy (FIG. 2).

### Example 5: Effect of GLP1 Peptide or GLP1 Peptide Variants and First Linker Peptides on In Vitro Human GLP1R Potency, Ex Vivo Human, and In Vivo Mouse Plasma Stability

Different GLP1 peptide or GLP1 peptide variants and different first linker peptides have different effects on in vitro hGLP1 receptor potency in an assay measuring cyclic adenosine monophosphate (cAMP) levels in hGLP1-overexpressing HEK cells. Fusions were screened for in vitro GLP1R potency in cell-based assays measuring intracellular cAMP production using the Lance competitive cAMP immunoassay (Perkin Elmer, Waltham, MA) according to the kit instructions. Clonal HEK293 cells stably expressing mouse or human GLP1R were used in the assays. Resulting data was used to calculate compound EC₅₀ values using Prism statistical software (GraphPad Software, San Diego, CA). In general, GLP1 variant (SEQ ID NO:1) is less potent than the other two GLP1 variants (SEQ ID NO:2 and SEQ ID NO:3) in the in vitro hGLP1R assay.

Ex vivo stability in human plasma was assessed. In brief, fresh, non-frozen human plasma was generated from heparinized blood by centrifugation. Fusion proteins were incubated in this matrix at 37° C. with gentle mixing for 0, 4, 24, 48, 72, and 96 hours. The stability of the GLP1 region of the fusion molecules over time in human plasma was monitored by immuno-affinity capture-trypsin digestion-LC-MS/MS analysis. Selected tryptic peptides, namely HSE (HSEGTFTSDVSSYLEGQAAK) (SEQ ID NO:76), HGE-1 (HGEGTFTSDVSSYLEEQAAK) (SEQ ID NO:77), and HGE-2 (HGEGTFTSDLSK) (SEQ ID NO:78), are located at the N-terminus of the GLP1 peptide or GLP1 variant peptides (SEQ ID NOs:1, 2, and 3), respectively. These selected tryptic peptides were monitored by LC-MS/MS after anti-GDF15 immuno-affinity capture and trypsin digestion which served as a surrogate measure of the concentration of fusion molecule with an intact GLP1 containing N-terminus. With this methodology, all the molecules tested demonstrated reasonable GLP1 stability over time in human plasma.

In vivo stability in mice was assessed. The fusion proteins were subcutaneously administered to male C57B1/6 mice at a dose of 2 mg/kg in PBS, pH 7. Blood samples were collected into K3E Sarstedt blood collection tubes with protease inhibitors at 0, 4, 24 and 48 hours post administration. Plasma was prepared by centrifugation. The stability of the GLP1 region of the fusion proteins over time in vivo in mice was monitored by immuno-affinity capture-trypsin digestion-LC-MS/MS analysis. Selected tryptic peptides, namely HSE (HSEGTFTSDVSSYLEGQAAK) (SEQ ID NO:76), HGE-1 (HGEGTFTSDVSSYLEEQAAK) (SEQ ID NO:77), and HGE-2 (HGEGTFTSDLSK) (SEQ ID NO:78), are located at the N-terminus of SEQ ID NOs:1, 2, and 3 containing fusion proteins, respectively. These selected tryptic peptides were monitored by LC-MS/MS after anti-GDF15 immuno-affinity capture and trypsin digestion which served as a surrogate measure of the concentration of fusion protein with an intact GLP1 containing N-terminus. With this methodology, all the molecules tested demonstrated reasonable GLP1 stability over time in vivo in mice.

Table 7 provides results for in vitro GLP1 receptor potency measuring cAMP levels of GLP1R over-expressing cells, ex vivo human plasma stability and in vivo mouse plasma stability, both assessed by mass spectrometry, for the indicated GLP1-GDF15 fusion proteins.

TABLE 7
In vitro hGLP1R potency, ex vivo human, and in vivo mouse plasma stability
of GLP1-GDF15 fusion proteins

| Molecule | | | In vitro | | Ex vivo human plasma | | | | | In vivo mouse plasma stability | | |
|---|---|---|---|---|---|---|---|---|---|---|---|---|
| | | First Linker | hGLP1R potency | | stability by MS | | | | | by MS (% intact GLP1 relative to | | |
| | GLP1 (SEQ | Peptide (SEQ | | +/− | (% intact GLP1 relative to t = 0) | | | | | HSA on the same molecule) | | |
| Seq ID | ID NO) | ID NO) | (nM) | error | 4 h | 24 h | 48 h | 72 h | 96 h | 4 h | 24 h | 48 h |
| 68 | 3 | 1-7 (11) | 0.09 | 0.04 | 90.8 | 94 | 89.5 | 74.4 | 70.1 | 93.4 | 57.6 | 81.5 |
| 60 | 3 | 1-6 (10) | 0.09 | 0.03 | Not tested | | | | | Not tested | | |
| 59 | 3 | 1-9 (13) | 0.1 | 0.05 | | | | | | | | |
| 64 | 3 | 1-19 (23) | 0.12 | 0.03 | 87.4 | 80 | 75.7 | 62.2 | 56.9 | 80.3 | 51.7 | 32.9 |
| 67 | 3 | 1-8 (12) | 0.13 | 0.02 | 86.5 | 89.8 | 76 | 68.2 | 64.7 | 88.8 | 55 | 34.1 |
| 70 | 2 | 1-7 (11) | 0.26 | 0.1 | 122.7 | 125.8 | 122.1 | 102.9 | 99.5 | 93.1 | 71.2 | 44.8 |
| 62 | 1 | 1-9 (13) | 0.35 | 0.1 | | | | | | Not tested | | |
| 69 | 2 | 1-8 (12) | 0.38 | 0.03 | 102.3 | 100.1 | 102.7 | 84.7 | 91.3 | 81.5 | 69.6 | 64.9 |
| 63 | 2 | 1-19 (23) | 0.39 | 0.15 | 92.5 | 88.7 | 82.2 | 77.8 | 77.3 | 109 | 95 | 74.6 |
| 61 | 1 | 1-6 (10) | 0.39 | 0.09 | Not tested | | | | | Not tested | | |
| 66 | 1 | 1-7 (11) | 0.6 | 0.14 | 107.5 | 99 | 91.2 | 80.5 | 70.7 | 112 | 55.9 | 37.3 |
| 71 | 1 | 1-19 (23) | 1.5 | 0.13 | 100.2 | 93.6 | 84.9 | 62.6 | 60.6 | 87 | 52 | 30 |
| 65 | 1 | 1-8 (12) | 2.98 | 0.74 | 100.7 | 84.9 | 84.8 | 69.4 | 165.4 | 102 | 59.3 | 45.8 |

### Example 6: Removing O-Linked Xylosylation by Eliminating Serine Residues in First and Second Linker Peptides

Molecules containing glycine-serine linkers were tested for O-linked xylose levels since xylose glycans were reported to attach to glycine-serine linkers (Spahr et. al, mAbs 6 (4): 904-914). Briefly, samples were prepared by dilution into Guanidine-HCl buffered at pH 8.0 for denaturation, followed by adding DTT for reduction and incubating for 1 hour at 37° C. After reduction, the samples were alkylated using freshly prepared iodoacetamide for 60 minutes at room temperature in the dark. Then, DTT was added to the sample to chelate unreacted iodoacetamide. Next, samples were desalted using Zeba Spin Desalting columns according to the manufacturer's protocol in 50 mM Tris, 1 mM CaCl₂, pH 8.0 and digested with trypsin (Promega) for 4 hr at 37° C. Following digestion, TFA was added to each sample in order to quench the digestion reaction. Digested samples were kept at 4° C. and injected into LC/MS within 24 hours.

Digested samples were injected into an Agilent AdvanceBio Peptide Map Micro Bore Rapid Resolution Column using an Agilent Infinity 1290 UHPLC (Agilent Technologies) at a flow rate of 0.1 mL/min. The column temperature was maintained at 65° C. Mass spectrometry grade HPLC solvents (0.1% Formic acid and B: 100% ACN in 0.1% Formic acid) were purchased from VWR. The proteolytic peptides were eluted from the column using a 50 min gradient of 2 to 40% ACN in 0.1% FA. The column effluent was introduced into a Thermo Orbitrap Q-Exactive Mass spectrometer via heated electrospray ionization probe (HESI) using a spray voltage of 3.5 kV, sheath gas 20, aux gas 7, ion transfer tube at 299° C. and vaporizer at 100° C.

A top 2 data dependent experiment was performed with the precursor scan set to Orbitrap detection, 70,000 resolution, mass range 150-2000 m/z, AGC Target 1.0e6, maximum injection time 50 ms, 1 microscan, positive polarity. The precursor decision criteria were monoisotopic precursor selection-peptides, charge state: 2-7, dynamic exclusion: 6.0 seconds and precursor intensity threshold of 5e4. Precursor peptides were isolated by the quadrupole with an isolation window of 1.6 m/z and sent to the collision cell. A collision energy of 28 resulted in high energy collisional dissociation (HCD) of the peptide. These fragments were then transferred to the orbitrap for mass measurement. The orbitrap settings were 17,500 resolution, 200-2000 m/z range, AGC target 5e5, maximum injection time 100 ms, 1 microscan, and spectra acquired in centroid mode.

Peptide mapping data is processed using Byonic (Protein Metrics Inc) search algorithm. Custom library of core xylose glycosoaminoglycans and their monoisotopic mass was added to the search parameters as a Ser specific modification. Byonic search results were imported into Byologic (Protein Metrics Inc) for quantification based on extracted ion chromatograms (XIC) areas of the modified and unmodified peptide species.

GLP1-GDF15 fusion proteins containing G₄S first and second linker peptides showed various levels of O-linked xylosylation on the serine residues of the first and second linker peptides. Table 8 shows the results of three such molecules expressed transiently from ExpiCHO cells. The level of xylose was protein context-dependent and ranged from ‘not detected’ to 1% in first linker peptides, which connect the GLP1 peptide or GLP1 peptide variants to the HSA protein, and from 11.61% to 56.2% in second linker peptides, which connect the HSA protein to the GDF15 protein or GDF15 protein variant. To avoid potential O-linked xylosylation risk on linker peptides, GLP1-GDF15 fusion proteins that do not contain serine residues in the first and/or second linker peptides were designed and generated. These GLP1-GDF15 fusion proteins contained linkers that comprise AP repeats, G₄A repeats or poly-glycine repeats.

TABLE 8
Levels of xylose detected on serine residues in first or second
linker peptide of GLP1-GDF15 fusion
proteins comprising G₄S linker peptides.

| | | | Total Xyl |
|---|---|---|---|
| | | | % relative |
| | | | abundance |
| Sequence | | | by peptide |
| ID | Linker motif | | mapping |
| 64 | Frist linker peptide (1-9) | SEQ ID NO: 13 | Not detected |
| | connecting GLP1 to HSA | AS-8x(G₄S) | |
| | Second linker peptide (2-3) | SEQ ID NO: 30 | 11.61 |
| | connecting HSA to GDF15 | GS-8x(G₄S) | |
| 67 | Frist linker peptide (1-8) | SEQ ID NO: 12 | 1.0 |
| | connecting GLP1 to HSA | AS-2x(G₄S) | |
| | Second linker peptide (2-3) | SEQ ID NO: 30 | 56.2 |
| | connecting HSA to GDF15 | GS-8x(G₄S) | |
| 63 | Frist linker peptide (1-19) | SEQ ID NO: 23 | 0.5 |
| | connecting GLP1 to HSA | 3x(G₄S) | |
| | Second linker peptide (2-3) | SEQ ID NO: 30 | 23.1 |
| | connecting HSA to GDF15 | GS-8x(G₄S) | |

### Example 7: In Vitro Potency on GLP1R and GDF15R

In vitro activity of GLP1-GDF15 fusion proteins were tested for both GLP1R and GDF15R potency in cell based assays. GDF15R (GFRAL) activity was determined by measuring phospho-AKT (Ser473) level in SK-N-AS human neuroblastoma cells stably transfected to overexpress either human or cynomolgus monkey GFRAL. Phosphorylation of AKT after treating the GFRAL expressing cells with various concentrations of fusion molecules was measured using the Phospho-AKT (Ser473) Assay kit (Cisbio, Beford, MA) according to manufacturer's instructions. Resulting data was used to calculate compound EC₅₀ values using Prism statistical software (GraphPad Software San Diego). GLP1-GDF15 fusion proteins tested in Table 9 have similar GDF15R potency, with EC50 in the 4.6-6.9 nM range.

GLP1R potency of the fusions was determined by measuring cAMP levels in clonal HEK293 cells stably transfected to overexpress human, mouse, or cynomolgus monkey GLP1R. Intracellular cAMP production after treating the cells with varying concentrations of the fusion molecules was measured using the Lance competitive cAMP immunoassay (Perkin Elmer, Waltham, Massachusetts) according to the kit instructions. Resulting data was used to calculate compound EC₅₀ values using Prism statistical software (GraphPad Software San Diego).

When serine residues in the first linker peptide, which connects the GLP1 peptide or GLP1 peptide variant to the HSA protein, were replaced with alanine or removed (switching from G₄S to G₄A or poly-G linker with similar length), GLP1 activity was not impacted when tested in a human or mouse GLP1R assay. In addition, changing the second linker peptide, which connects the HSA protein to the GDF15 protein or GDF15 protein variant, from GS-8x(G₄S) to GA-8x(G₄A) or 10x(AP) did not impact GLP1R potency. In addition, fusion proteins with increasing numbers of repeats in first linker (5X(G₄A), 8X(G₄A), 5X(AP), 10X(AP), 20X(AP), 25X(AP) were made and tested in the human GLP1R assay. Results demonstrated that these fusion proteins activated GLP1R signaling with varying potency.

TABLE 9
in vitro potency of GLP1-GDF15 fusion proteins on GLP1R and GDF15R

| | cAMP induction | | | | | | | | |
|---|---|---|---|---|---|---|---|---|---|
| | hGLP1R | | | mGLP1R | | | cGLP1R | | |
| SEQ ID | EC50 | | | EC50 | | | EC50 | | |
| NO: | (nM) | STD | n | (nM) | STD | n | (nM) | STD | n |
| 45 | 0.070 | 0.012 | 2 | 0.023 | 0.003 | 2 | 0.155 | 0.125 | 4 |
| 47 | 0.081 | 0.038 | 2 | 0.029 | 0.007 | 2 | 0.156 | 0.074 | 4 |
| 48 | 0.092 | 0.040 | 2 | 0.037 | 0.006 | 2 | 0.163 | 0.038 | 4 |
| 49 | 0.127 | 0.058 | 2 | 0.040 | 0.001 | 2 | 0.170 | 0.031 | 4 |
| 50 | 0.095 | 0.050 | 2 | 0.029 | 0.004 | 2 | 0.154 | 0.029 | 4 |
| 33 | 0.120 | 0.051 | 2 | 0.034 | 0.000 | 2 | 0.190 | 0.140 | 4 |
| 46 | 0.065 | 0.008 | 2 | 0.030 | 0.001 | 2 | 0.117 | 0.003 | 2 |
| 44 | 0.076 | 0.037 | 2 | 0.032 | 0.004 | 2 | 0.109 | 0.039 | 2 |
| GLP1 | 0.011 | 0.004 | 25 | 0.012 | 0.004 | 21 | 0.013 | 0.003 | 8 |
| Dulaglutide | 0.038 | 0.010 | 27 | 0.020 | 0.004 | 23 | 0.045 | 0.014 | 8 |
| Liraglutide | 0.057 | 0.025 | 18 | 0.052 | 0.022 | 14 | 0.079 | 0.022 | 2 |
| Albiglutide | 0.981 | 0.438 | 23 | 1.486 | 0.624 | 19 | 1.759 | 0.735 | 6 |

| | Phosphor-AKT (Ser473) | | | | | |
|---|---|---|---|---|---|---|
| | hGDF15R | | | cGDF15R | | |
| SEQ ID | EC50 | | | EC50 | | |
| NO: | (nM) | 95% Cl | n | (nM) | 95% Cl | n |
| 45 | 4.561 | 3.975-5.258 | 1 | 1.031 | 0.6679-1.4 | 1 |
| 47 | 5.899 | 5.146-6.813 | 1 | 0.937 | 0.7413-1.137 | 1 |
| 48 | 6.283 | 5.479-7.281 | 1 | 1.585 | 1.374-1.819 | 1 |
| 49 | 4.825 | 4.11-5.726 | 1 | 1.157 | 0.7357-1.62 | 1 |
| 50 | 5.983 | 5.333-6.731 | 1 | 1.225 | 0.9865-1.473 | 1 |
| 33 | 5.251 | 4.523-6.113 | 1 | 1.004 | 0.7745-1.243 | 1 |
| 46 | 6.917 | 6.3-7.623 | 1 | 1.521 | 1.267-1.805 | 1 |
| 44 | 4.945 | 4.393-5.572 | 1 | 1.350 | 1.102-1.614 | 1 |

### Example 8: Effect of First and Second Linker Peptides on Protein Purity and Stability

Size exclusion high-performance liquid chromatography (SE-HPLC) was used to examine the purity and stability of the molecules by quantifying the percentage of main species as well as both high molecular weight (HMW) species that represent aggregates and low molecular weight (LMW) species that represent fragments. Briefly, to determine protein purity, 20 μg of protein was injected onto Tosoh TSKgel BioAssist G3SWXL (Cat #20026) column with a 1×DPBS, pH 7.2 (Gibco Cat #14190-136) mobile phase. The protein species were eluted at a flow rate of 1 mL/min at room temperature, and the UV-280 nm absorbance values were monitored using a Dionex Ultimate3000 HPLC system equipped with a variable wavelength detector. To determine protein stability, 100 μg of protein was injected onto Tosoh TSKgel BioAssist G3SWXL (Cat #20026) column with a 0.2 M sodium phosphate pH 7.0 mobile phase. The protein species were eluted at a flow rate of 0.7 mL/min at room temperature, and the UV-280 nm absorbance values were monitored using a Dionex Ultimate3000 HPLC system equipped with a variable wavelength detector. Data is analyzed using Chromeleon software.

When GLP1-GDF15 fusion proteins were made from transient expression in ExpiCHO cells and purified by affinity capture following the methods described in Example 2, the GLP1-GDF15 fusion proteins with 10x(AP) second linker peptides (SEQ ID NO:28) (connecting the HSA protein to the GDF15 protein or GDF15 protein variant) consistently resulted in a lower percentage of low molecular weight (LMW) species compared with molecules containing GA-8x(G₄A) second linker peptides (SEQ ID NO:29) (Table 10). The LMW species were removed by polishing steps during purification and the resulting final GLP1-GDF15 fusion proteins were greater than 97% pure by SE-HPLC.

These GLP1-GDF15 fusion proteins were tested for stability under defined stressed conditions. To force chemical induced oxidation stress, the samples were exposed to a final concentration of 0.1% hydrogen peroxide and incubated for 6 hours in the dark prior to adding catalase to stop the reaction. In the metal induced oxidation condition, a final concentration of 30 μM ferrous iron was added to the samples. After incubation for two weeks in the dark, EDTA was added to stop the reaction. To test stability at low pH, the samples were dialyzed into 50 mM acetate pH 3.5 buffer, kept for 6 hours and dialyzed back to 0.1M sodium phosphate pH 7.4. To test sample stability under thermal stress, they were concentrated to approximately 10 mg/ml using ultra centrifugal filter with a molecular weight cut-off of 30 KDal and were held at 40° C. for two weeks in PBS. These samples that undergo the abovementioned chemical- or metal-induced oxidation, as well as low pH condition or thermal stress condition were analyzed under analytical size-exclusion chromatography (TOSOH column) with flow rate of 1 ml/min for a 20-minute run at room temperature. Signals are collected for UV-280 nm (Agilent 1100 LC system) and data analysis is done with Chemstation (Agilent).

Table 11 shows the influence of second linker on the stability of GLP1-GDF15 fusion proteins under thermal stress (40° C. for 2 weeks). The GLP1-GDF15 fusion proteins that contained a 10x(AP) second linker peptide (SEQ ID NO:28) resulted in a lower level of LMW species formed compared with GLP1-GDF15 fusion proteins containing a GA-8x(G₄A) second linker peptide (SEQ ID NO:29), indicating that a AP second linker peptide is more thermally stable compared with a G₄A second linker peptide. Under other tested stress conditions (low pH, metal- or chemical-induced oxidation), the level of LMW species of the samples remain at minimal levels, similar to the non-stressed samples.

Differential Scanning Calorimetry (DSC) was performed to determine the thermal stability of the GLP1-HSA-GDF15 proteins. Samples were evaluated at approximately 0.5-1 mg/ml in PBS pH 7.4 buffer using an automated MicroCal VP-Capillary DSC instrument. The thermal scans span from 25° C. to 95° C. at a linear rate of 1° C./min. A pre-scan time of 15 minutes and a filtering period of 10 seconds were used for each run. The data were processed using non-2 state fitting function in Origin7 software package. DSC results demonstrated that these GLP1-GDF15 fusion proteins have melting temperature (Tₘ) in the 63-72° C. range (Table 12), similar to an HSA-GDF15 fusion protein that does not contain a GLP1 peptide or GLP1 peptide variant.

TABLE 10
GLP1-GDF15 fusion proteins with different first and second linker
peptides show various levels of low molecular weight species by
SE-HPLC after affinity purification.

| SEQ | First linker | Second linker | SE-HPLC after affinity | | |
|---|---|---|---|---|---|
| ID | peptide | peptide | purification | | |
| NO: | (SEQ ID NO) | (SEQ ID NO) | % HMW | % main | % LMW |
| 45 | 10x(AP) (6) | 10x(AP) (28) | 0-1.32 | 98.68-100 | 0 |
| 33 | 5x(G₄A) (18) | 10x(AP) (28) | 0 | 100 | 0 |
| 44 | 15x(AP) (5) | 10x(AP) (28) | 0 | 100 | 0 |
| 47 | 25x(G) (22) | GA-8x(G₄A) | 2.3 | 86.64 | 11.06 |
| | | (29) | | | |
| 48 | 20x(G) (21) | GA-8x(G₄A) | 5.56 | 83.77 | 10.67 |
| | | (29) | | | |
| 49 | 4x(G₄A) (17) | GA-8x(G₄A) | 2.93 | 94.64 | 2.43 |
| | | (29) | | | |
| 46 | 5x(AP) (5) | GA-8x(G₄A) | 0 | 92.33 | 7.67 |
| | | (29) | | | |
| 50 | 5x(G₄A) (18) | GA-8x(G₄A) | 0.77-3 | 89.71-91.00 | 7.29-8.22 |
| | | (29) | | | |

TABLE 11
GLP1-GDF15 fusion proteins with different first
and second linker peptides show various levels of low
molecular weight species under thermal stress (40° C. for 2 weeks).

| | | | | SE-HPLC | | | |
|---|---|---|---|---|---|---|---|
| | GLP1 peptide | First linker | Second linker | after all-step | SE-HPLC after 40° C. | | |
| SEQ ID | or GLP1 | peptide (SEQ | peptide (SEQ | purification | for 2 weeks | | |
| NO: | peptide variant | ID NO:) | ID NO:) | % main | % HMW | % main | % LMW |
| 45 | GLP1-based | 10x(AP) (6) | 10x(AP) (28) | 98.68-100 | 5.32 | 92.6 | 2.13 |
| 33 | GLP1-based | 5x(G₄A) (18) | 10x(AP) (28) | 100 | 2.5 | 95.4 | 2.1 |
| 44 | Exendin4-based | 5x(AP) (5) | 10x(AP) (28) | 100 | 7.3 | 90.8 | 2.6 |
| 47 | GLP1-based | 25x(G) (22) | GA-8x(G₄A) (29) | 100 | 0.91 | 93.9 | 5.19 |
| 48 | GLP1-based | 20x(G) (21) | GA-8x(G₄A) (29) | 97.13 | 5.22 | 86.97 | 7.81 |
| 49 | GLP1-based | 4x(G₄A) (17) | GA-8x(G₄A) (29) | 100 | 0.42 | 93.56 | 6.02 |
| 50 | GLP1-based | 5x(G₄A) (18) | GA-8x(G₄A) (29) | 100 | 0.49 | 92.17 | 7.34 |
| 46 | Exendin4-based | 5x(AP) (5) | GA-8x(G₄A) (29) | 100 | 5.9 | 74.5 | 19.6 |

TABLE 12
Tm for GLP1-GDF15 fusion proteins by differential
scanning calorimetry (DSC).

| SEQ | GLP1 peptide | First linker | Second linker | | | | |
|---|---|---|---|---|---|---|---|
| ID | or GLP1 | peptide (SEQ | peptide (SEQ ID | Tm by DSC (° C.) | | | |
| NO: | peptide variant | ID NO:) | NO:) | Tm1 | Tm2 | Tm3 | Tm4 |
| 45 | GLP1-based | 10x(AP) (6) | 10x(AP) (28) | 64.88 | 68.79 | 73.38 | 76.59 |
| 33 | GLP1-based | 5x(G₄A) (18) | 10x(AP) (28) | 63.96 | 68.38 | 73.37 | 76.44 |
| 47 | GLP1-based | 25x(G) (22) | GA-8x(G₄A) (29) | 68.31 | 74.55 | 77.98 | na |
| 48 | GLP1-based | 20x(G) (21) | GA-8x(G₄A) (29) | 64.74 | 69.28 | 74.27 | 77.15 |
| 49 | GLP1-based | 4x(G₄A) (17) | GA-8x(G₄A) (29) | 64.64 | 69.58 | 74.29 | 77.04 |
| 50 | GLP1-based | 5x(G₄A) (18) | GA-8x(G₄A) (29) | 63.39 | 67.78 | 72.8 | 75.99 |
| 46 | Exendin4-based | 5x(AP) (5) | GA-8x(G₄A) (29) | 72.07 | 74.81 | 77.02 | na |
| 44 | Exendin4-based | 5x(AP) (5) | 10x(AP) (29) | 69.79 | 73.82 | 76.23 | na |

### Example 9: Ex Vivo Human Plasma Study

The stability of the GLP1-GDF15 fusion proteins was assessed ex vivo in human plasma. In brief, fresh, non-frozen human plasma was generated from heparinized blood by centrifugation. Fusion proteins were incubated in this matrix at 37° C. with gentle mixing for 0, 4, 24, 48, 72 and 96 hours. The concentration of the fusion molecules over time in human plasma was monitored by immuno-affinity capture with an anti-GDF15 antibody and immuno-detection with either an anti-HSA antibody (“total format”) or anti-GLP1 N-terminal specific antibody (“GLP1 active format”). Table 13 and 14 demonstrate the results from these two immunoassays.

TABLE 13
ex vivo human plasma stability by immunoassay measured by an anti-
GDF15 capture antibody and an anti-HSA detection antibody.

| | Concentration (μg/mL) | | | | | | | Normalized % recovery to 0 hr | | | | | | |
|---|---|---|---|---|---|---|---|---|---|---|---|---|---|---|
| Time | SEQ | SEQ | SEQ | SEQ | SEQ | SEQ | SEQ | SEQ | SEQ | SEQ | SEQ | SEQ | SEQ | SEQ |
| point | ID: | ID: | ID: | ID: | ID: | ID: | ID: | ID: | ID: | ID: | ID: | ID: | ID: | ID: |
| (hr) | 50 | 33 | 49 | 48 | 47 | 45 | 44 | 50 | 33 | 49 | 48 | 47 | 45 | 44 |
| 0 | 13.69 | 13.13 | 13.14 | 12.69 | 13.32 | 12.79 | 13.77 | 100.0 | 100.0 | 100.0 | 100.0 | 100.0 | 100.0 | 100.0 |
| 4 | 14.14 | 13.56 | 12.83 | 13.20 | 12.53 | 12.89 | 13.30 | 103.3 | 103.3 | 97.6 | 104.0 | 94.0 | 100.8 | 96.6 |
| 24 | 13.84 | 13.49 | 12.16 | 13.21 | 13.3 | 12.76 | 12.53 | 101.1 | 102.8 | 92.5 | 104.0 | 99.9 | 99.8 | 91.0 |
| 48 | 12.84 | 13.57 | 12.21 | 12.56 | 12.48 | 11.88 | 11.55 | 93.8 | 103.4 | 92.9 | 98.9 | 93.7 | 92.9 | 83.9 |
| 72 | 13.18 | 14.00 | 11.21 | 12.12 | 12.35 | 12.18 | 12.93 | 96.3 | 106.7 | 85.3 | 95.5 | 92.7 | 95.2 | 93.9 |
| 96 | 12.92 | 13.01 | 11.23 | 12.09 | 12.16 | 12.32 | 12.34 | 94.4 | 99.1 | 85.4 | 95.2 | 91.3 | 96.3 | 89.6 |

TABLE 14
ex vivo human plasma stability by immunoassay by an anti-GDF15 capture
antibody and an anti-GLP1 detection antibody (recognizing the
N-terminal GLP1, specific to the active form of GLP1).

| | Concentration (μg/mL) | | | | | | | Normalized % recovery to 0 hr | | | | | | |
|---|---|---|---|---|---|---|---|---|---|---|---|---|---|---|
| Time | SEQ | SEQ | SEQ | SEQ | SEQ | SEQ | SEQ | SEQ | SEQ | SEQ | SEQ | SEQ | SEQ | SEQ |
| point | ID: | ID: | ID: | ID: | ID: | ID: | ID: | ID: | ID: | ID: | ID: | ID: | ID: | ID: |
| (hr) | 50 | 33 | 49 | 48 | 47 | 45 | 44 | 50 | 33 | 49 | 48 | 47 | 45 | 44 |
| 0 | 11.45 | 10.93 | 11.28 | 11.54 | 11.50 | 11.99 | 12.04 | 100.0 | 100.0 | 100.0 | 100.0 | 100.0 | 100.0 | 100.0 |
| 4 | 11.60 | 10.86 | 10.85 | 11.44 | 11.00 | 11.35 | 10.88 | 101.3 | 99.4 | 96.2 | 99.1 | 95.7 | 94.6 | 90.4 |
| 24 | 10.32 | 10.50 | 10.17 | 10.43 | 10.08 | 10.64 | 9.73 | 90.2 | 96.1 | 90.2 | 90.4 | 87.7 | 88.7 | 80.8 |
| 48 | 9.07 | 9.66 | 9.47 | 9.40 | 8.28 | 10.26 | 8.95 | 79.2 | 88.4 | 83.9 | 81.5 | 72.0 | 85.6 | 74.3 |
| 72 | 8.68 | 9.22 | 8.71 | 8.76 | 6.30 | 9.17 | 9.13 | 75.8 | 84.4 | 77.3 | 75.9 | 54.8 | 76.4 | 75.9 |
| 96 | 8.37 | 9.71 | 7.94 | 8.24 | 6.62 | 9.14 | 8.21 | 73.1 | 88.8 | 70.4 | 71.4 | 57.6 | 76.2 | 68.2 |

### Example 10: Multispecies Pharmacokinetics

### Mouse Pharmacokinetics

GLP1-GDF15 fusion proteins derived from SEQ ID NOs:50, 45, 46, and 44 were administered to female C57B1/6 mice at a dose of 5 mg/kg IV and SC in PBS, pH 7. Blood samples were collected, plasma was processed and drug concentrations were measured up to 4 days following both routes of administration. The concentration of analytes in plasma after IV and SC administration was measured by immuno-affinity capture-trypsin digestion-LC-MS/MS analysis. Selected tryptic peptides, namely, ALV (ALVLIAFAQYLQQSPFEDHVK) (SEQ ID NO:79), HGE-1 (HGEGTFTSDVSSYLEEQAAK) (SEQ ID NO:77), and HGE-2 (HGEGTFTSDLSK) (SEQ ID NO:78), and TDT (TDTGVSLQTYDDLLAK) (SEQ ID NO:80), which are located near the N-terminus of the HSA protein, the N-terminus of the GLP1 peptide or GLP1 peptide variant, and the C-terminus of the GDF15 protein or GDF15 protein variant, respectively. Monitoring these surrogate peptides enabled pharmacokinetic assessment of each region (GLP1, HSA, GDF15) of the GLP1-GDF15 fusion proteins. The plasma drug concentration-time profiles are summarized in Tables 15-22.

TABLE 15
Plasma concentration (μg/mL) and standard error of the mean
(SEM, n = 3) of SEQ ID NO: 50 over time following a single
subcutaneous (SC) administration in C57BL/6 female mice.

| SC | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 2 | 14.780 | 15.140 | 16.360 | 0.905 | 1.014 | 1.063 |
| 6 | 26.700 | 37.300 | 30.180 | 1.248 | 5.012 | 1.563 |
| 24 | 19.980 | 36.780 | 28.880 | 0.691 | 2.956 | 0.944 |
| 48 | 10.716 | 22.940 | 19.640 | 0.330 | 1.677 | 0.458 |
| 72 | 5.034 | 10.790 | 12.960 | 0.286 | 0.525 | 0.631 |
| 96 | 2.450 | 7.218 | 8.444 | 0.244 | 0.844 | 0.699 |

TABLE 16
Plasma concentration (μg/mL) and standard error of the mean (SEM, n = 3)
of SEQ ID NO: 50 over time following a single intravenous
(IV) administration in C57BL/6 female mice.

| IV | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 0.5 | 68.320 | 71.760 | 82.860 | 4.188 | 6.659 | 5.408 |
| 6 | 45.020 | 48.560 | 60.660 | 1.427 | 1.487 | 1.707 |
| 24 | 16.900 | 23.820 | 28.340 | 0.991 | 0.913 | 1.646 |
| 48 | 9.472 | 20.620 | 20.540 | 0.510 | 0.843 | 1.365 |
| 72 | 5.048 | 12.118 | 14.440 | 0.303 | 0.996 | 0.842 |
| 96 | 2.144 | 5.404 | 7.926 | 0.103 | 0.365 | 0.445 |

TABLE 17
Plasma concentration (μg/mL) and standard error of the mean (SEM, n = 3)
of SEQ ID NO: 45 over time following a single subcutaneous
(SC) administration in C57BL/6 female mice.

| SC | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 2 | 13.536 | 12.032 | 12.512 | 3.674 | 3.373 | 3.499 |
| 6 | 21.780 | 20.540 | 22.120 | 3.155 | 2.770 | 3.161 |
| 24 | 18.020 | 27.580 | 26.520 | 1.712 | 3.014 | 2.732 |
| 48 | 8.856 | 20.060 | 18.980 | 0.469 | 1.183 | 1.248 |
| 72 | 4.348 | 18.180 | 15.260 | 0.329 | 1.697 | 1.065 |
| 96 | 1.786 | 9.866 | 9.676 | 0.164 | 0.907 | 0.854 |

TABLE 18
Plasma concentration (μg/mL) and standard error of the mean (SEM,
n = 3) of SEQ ID NO: 45 over time following a single
intravenous (IV) administration in C57BL/6 female mice.

| IV | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 0.5 | 76.800 | 69.080 | 69.560 | 7.469 | 7.052 | 7.981 |
| 6 | 56.180 | 57.040 | 59.460 | 5.162 | 5.781 | 5.527 |
| 24 | 21.120 | 30.860 | 30.680 | 1.668 | 2.444 | 2.480 |
| 48 | 10.398 | 22.560 | 21.500 | 0.855 | 2.104 | 2.152 |
| 72 | 4.794 | 18.800 | 16.180 | 0.368 | 1.880 | 1.030 |
| 96 | 1.986 | 10.854 | 10.284 | 0.134 | 0.837 | 0.707 |

TABLE 19
Plasma concentration (μg/mL) and standard error of the mean (SEM, n = 3)
of SEQ ID NO: 46 over time following a single subcutaneous
(SC) administration in C57BL/6 female mice.

| SC | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 2 | 3.420 | 2.850 | 3.720 | 0.376 | 0.311 | 0.337 |
| 6 | 5.718 | 6.522 | 7.516 | 0.730 | 0.704 | 0.609 |
| 24 | 4.792 | 8.504 | 9.154 | 0.398 | 0.708 | 0.858 |
| 48 | 2.146 | 5.766 | 6.910 | 0.389 | 0.541 | 0.455 |
| 72 | 0.689 | 2.818 | 3.286 | 0.061 | 0.260 | 0.336 |
| 96 | 0.205 | 1.270 | 1.516 | 0.026 | 0.137 | 0.186 |

TABLE 20
Plasma concentration (μg/mL) and standard error
of the mean (SEM, n = 3) of SEQ ID NO: 46 following a single
intravenous (IV) administration in C57BL/6 female mice.

| IV | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 0.5 | 55.525 | 42.525 | 57.875 | 15.521 | 10.592 | 15.368 |
| 6 | 27.125 | 24.750 | 34.950 | 5.185 | 4.660 | 6.461 |
| 24 | 4.933 | 9.423 | 11.280 | 0.255 | 0.893 | 0.879 |
| 48 | 1.355 | 5.023 | 5.893 | 0.125 | 0.278 | 0.702 |
| 72 | 0.510 | 2.623 | 2.968 | 0.049 | 0.269 | 0.329 |
| 96 | 0.196 | 1.345 | 1.593 | 0.020 | 0.155 | 0.171 |

TABLE 21
Plasma concentration (μg/mL) and standard error of the
mean (SEM, n = 3) of SEQ ID NO: 44 following a single
subcutaneous (SC) administration in C57BL/6 female mice.

| SC | Concentration (μg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 2 | 6.340 | 6.124 | 6.792 | 2.029 | 1.845 | 2.289 |
| 6 | 8.534 | 10.310 | 10.672 | 1.697 | 1.721 | 1.876 |
| 24 | 4.646 | 10.098 | 10.018 | 0.969 | 1.801 | 1.874 |
| 48 | 1.879 | 6.264 | 6.480 | 0.486 | 1.171 | 1.162 |
| 72 | 0.691 | 3.678 | 3.694 | 0.153 | 0.766 | 0.760 |
| 96 | 0.194 | 1.758 | 1.860 | 0.048 | 0.304 | 0.331 |

TABLE 22
Plasma concentration (μg/mL) and standard error of the mean
(SEM, n = 3) of SEQ ID NO: 44 following a single
intravenous (IV) administration in C57BL/6 female mice.

| IV | Concentration (mg/mL) | | | SEM | | |
|---|---|---|---|---|---|---|
| Time | HGE | ALV | TDT | HGE | ALV | TDT |
| (h) | (GLP1) | (HSA) | (GDF15) | (GLP1) | (HSA) | (GDF15) |
| 0.5 | 57.920 | 53.300 | 60.760 | 4.648 | 3.971 | 4.489 |
| 6 | 29.560 | 33.360 | 37.400 | 2.266 | 2.199 | 2.176 |
| 24 | 4.564 | 10.420 | 10.684 | 0.424 | 0.393 | 0.560 |
| 48 | 1.300 | 6.276 | 6.022 | 0.217 | 0.312 | 0.250 |
| 72 | 0.540 | 3.396 | 3.562 | 0.040 | 0.257 | 0.195 |
| 96 | 0.159 | 1.784 | 1.804 | 0.025 | 0.180 | 0.123 |

Noncompartmental pharmacokinetic analysis (NCA) using the data in Tables 15-22 revealed a terminal half-life of 14-23 hours for the GLP1 peptide or GLP1 peptide variant, 22-49 hours for the GDF15 protein or GDF15 protein variant, and 22-47 hours for the HSA protein following SC and IV administration of the four GLP1-GDF15 fusion proteins tested in C57BL/6 mice (Table 23).

TABLE 23
Estimated terminal half-life of GLP1-GDF15
fusion proteins upon 5 mg/kg IV and SC
administration in female C57BL/6 mice.

| SEQ | | Half-life (hour) | Half-life (hour) |
|---|---|---|---|
| ID | | NCA SC | NCA IV |
| NO: | Region | Dosing | Dosing |
| 50 | GDF15 | 39.4 | 39.8 |
| 50 | GLP1 | 22.5 | 24.4 |
| 50 | HSA | 29.5 | 24.8 |
| 45 | GDF15 | 49.4 | 46.7 |
| 45 | GLP1 | 21.8 | 20.1 |
| 45 | HSA | 46.9 | 38.3 |
| 46 | GDF15 | 21.9 | 25.4 |
| 46 | GLP1 | 14.2 | 17.2 |
| 46 | HSA | 22.0 | 25.3 |
| 44 | GDF15 | 26.7 | 28.4 |
| 44 | GLP1 | 15.8 | 15.2 |
| 44 | HSA | 28.8 | 26.5 |

### Monkey Pharmacokinetics

GLP1-GDF15 fusion proteins derived from SEQ ID NOs:33, 50, 45, 46, and 44 were administered to naïve male cynomolgus monkeys (Macaca fascicularis) at a dose of 0.5 mg/kg SC in PBS, pH 7. Blood samples were collected, plasma was processed and drug concentrations were measured up to 21 days using immunoassay bioanalysis. Immunoassay strategy included an anti-GDF15 capture antibody and detection with either an antibody recognizing intact GLP1 (“Active”) or an antibody recognizing HSA (“Total”). The plasma drug concentration-time profile is summarized in Tables 24 and 25.

TABLE 24
Average (Avg, n = 3) plasma concentration (ng/ml) of active SEQ ID
NOs: 33, 50, 45, 46, and 44 over time following a single SC
administration in cynomolgus monkeys as determined by immunoassay.

| | Time | SEQ ID | SEQ ID | SEQ ID | SEQ ID | SEQ ID |
|---|---|---|---|---|---|---|
| | point | NO: 46 | NO: 44 | NO: 45 | NO: 50 | NO: 33 |
| Active | Predose | <2.50 | <2.50 | <2.50 | <2.50 | <1.25 |
| GLP1 | 6-hr | 1747.72 | 2530.81 | 2457.83 | 1667.36 | 1880.02 |
| (ng/mL) | 24-hr | 3057.60 | 3492.18 | 3946.41 | 3191.24 | 3151.63 |
| | 48-hr | 3177.96 | 3416.86 | 3655.95 | 3643.12 | 3448.62 |
| | 72-hr | 2689.49 | 3013.14 | 3015.11 | 2991.62 | 2949.14 |
| | 120-hr | 1501.85 | 1669.39 | 1402.39 | 2029.62 | 1991.88 |
| | 168-hr | 813.84 | 1020.04 | 725.75 | 1208.07 | 1246.28 |
| | 240-hr | 378.81 | 471.08 | 265.28 | 564.57 | 583.56 |
| | 336-hr | 137.73 | 192.90 | 81.18 | 197.74 | 211.92 |
| | 432-hr | 53.16 | 61.11 | 25.47 | 70.28 | 79.78 |
| | 528-hr | 23.10 | 35.96 | 10.60 | 25.70 | 36.59 |

TABLE 25
Average (Avg, n = 3) plasma concentration (ng/ml) of Total SEQ ID
NOs: 33, 50, 45, 46, and 44 over time following a single SC
administration in cynomolgus monkeys as determined by immunoassay.

| | Time | SEQ ID | SEQ ID | SEQ ID | SEQ ID | SEQ ID |
|---|---|---|---|---|---|---|
| | point | NO: 46 | NO: 44 | NO: 45 | NO: 50 | NO: 33 |
| Total | Predose | <5.00 | <5.00 | <5.00 | <5.00 | <5.00 |
| (ng/mL) | 6-hr | 1628.27 | 2299.98 | 2139.00 | 1427.28 | 1726.39 |
| | 24-hr | 3350.99 | 3873.18 | 4639.00 | 3430.23 | 3451.14 |
| | 48-hr | 3956.92 | 4427.70 | 5607.40 | 4892.71 | 4542.34 |
| | 72-hr | 3672.20 | 4399.17 | 5310.44 | 4303.43 | 4514.28 |
| | 120-hr | 2699.22 | 3612.21 | 4616.56 | 4133.96 | 4114.96 |
| | 168-hr | 1930.94 | 2745.57 | 3902.73 | 3237.20 | 3431.95 |
| | 240-hr | 1261.95 | 1964.84 | 2820.20 | 2353.01 | 2504.95 |
| | 336-hr | 719.72 | 1357.09 | 2005.69 | 1597.70 | 1682.87 |
| | 432-hr | 442.57 | 806.98 | 1399.11 | 964.85 | 1096.51 |
| | 528-hr | 328.35 | 532.75 | 961.88 | 708.00 | 879.52 |

Pharmacokinetic analysis of the data in Tables 24 and 25 revealed a terminal half-life of 2-3 days for the active molecule and 5-8 days for the total molecule in cynomolgus monkeys following SC administration (Table 26).

TABLE 26
Terminal half-life of SEQ ID NOs: 50, 33,
45, 46, and 44 following 0.5 mg/kg SC
administration in cynomolgus monkeys.

| SEQ | | |
|---|---|---|
| ID | | half-life |
| NO: | Detection | (d) |
| 44 | Active | 3.2 |
| | Total | 6.6 |
| 46 | Active | 2.6 |
| | Total | 4.6 |
| 50 | Active | 2.9 |
| | Total | 6.6 |
| 45 | Active | 2.3 |
| | Total | 7.6 |
| 33 | Active | 3.0 |
| | Total | 7.8 |

### Example 11: In Vivo Potency of GLP1-GDF15 Fusion Proteins

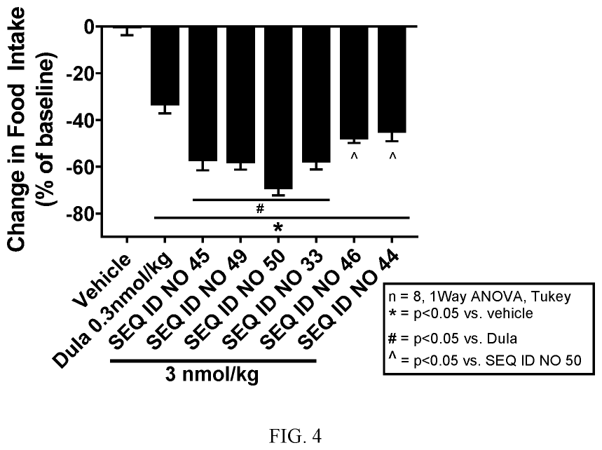

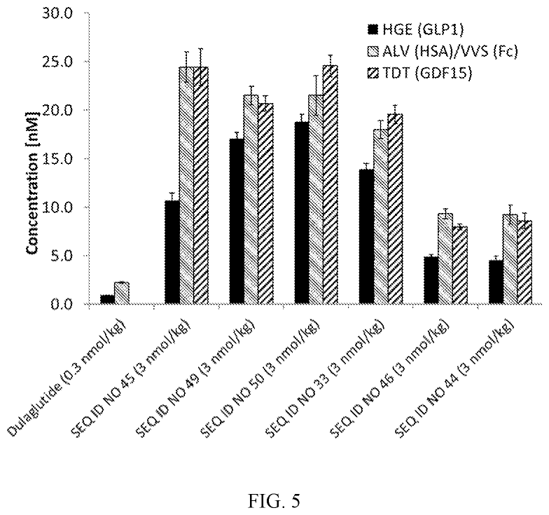

Assessment of food intake in C57BL/6 male mice: The purpose of this experiment was to demonstrate the in vivo pharmacodynamic effect of molecules corresponding to SEQ ID NOs:50, 33, 49, 45, 46, and 44 on the inhibition of food intake in C57BL/6 mice. 24-hour food intake was measured before and after subcutaneous administration of GLP1-GDF15 fusion proteins (test articles) or controls (PBS vehicle or dulaglutide) between 4:00 and 5:00 pm. The change in food weight for each cage was recorded every 24 hours. The results are expressed as the percent change in food intake compared to the 24 hours prior to treatment and are partially dependent on the circulating concentration of each test article used in the study (FIG. 4). Circulating concentration of test articles was determined 24 hours after administration by immuno-affinity capture-trypsin digestion-LC-MS/MS analysis. Selected tryptic peptides were monitored, namely, ALV (ALVLIAFAQYLQQSPFEDHVK) (SEQ ID NO:79), HGE-1 (HGEGTFTSDVSSYLEEQAAK) (SEQ ID NO:77), and HGE-2 (HGEGTFTSDLSK) (SEQ ID NO:78), and TDT (TDTGVSLQTYDDLLAK) (SEQ ID NO:80), which are located near the N-terminus of the HSA protein, the N-terminus of the GLP1 peptide or GLP1 peptide variant, and the C-terminus of the GDF15 protein or GDF15 protein variant, respectively. The VVS peptide (VVSVLTVLHQDWLNGK) (SEQ ID NO:82) is located in the Fc portion of dulaglutide. Monitoring these surrogate peptides enabled pharmacokinetic assessment of each region (GLP1, HSA, GDF15) of the GLP1-GDF15 fusion proteins. The plasma drug concentrations are shown in FIG. 5. The results indicated that subcutaneous administration of SEQ ID NOs:50, 33, 49, 45, 46, and 44 to C57BL/6 mice significantly inhibited food intake relative to vehicle-treated animals.

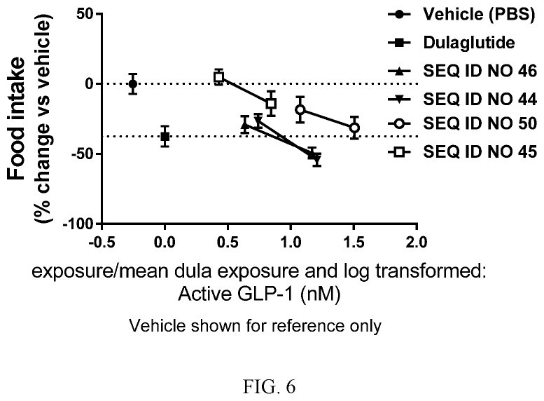

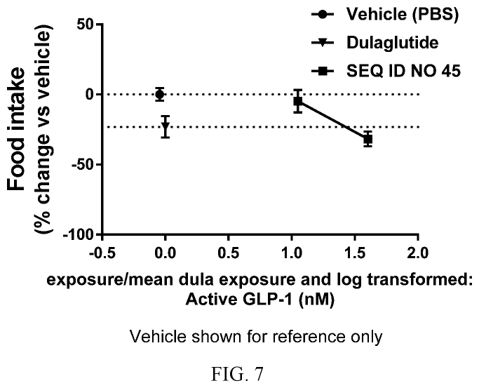

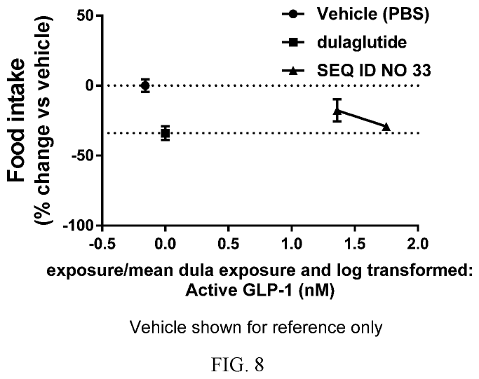

Assessment of GLP1R mediated food intake effects in GFRAL KO mice: Inhibition of food intake in C57BL/6 mice is a result of both GLP1R and GDF15R (GFRAL) engagement. To determine the GLP1R specific effects of SEQ ID NOs:50, 33, 45, 46, and 44 on food intake, mice lacking GFRAL were used. The objective of these experiments was to determine the extent of GLP1R engagement by the fusion proteins when they were administered at a dose predicted to be efficacious for HSA-GDF15 fusions in mice (FIGS. 1 and 2 and US20170327560A1). 24-hour food intake was measured before and after subcutaneous administration of GLP1-GDF15 fusion proteins (test articles: 3 and 10 nmol/kg) or controls (PBS vehicle or dulaglutide) between 4:00 and 5:00 pm. The change in food weight for each cage was recorded every 24 hours. Circulating concentration of fusion molecules containing intact GLP1 peptide or GLP1 peptide variants was determined 24 hours after administration by immuno-affinity capture-trypsin digestion-LC-MS/MS analysis and selective monitoring of tryptic peptides, HGE-1 (HGEGTFTSDVSSYLEEQAAK) (SEQ ID NO:77) or HGE-2 (HGEGTFTSDLSK) (SEQ ID NO:78) which are located near the N-terminus of the GLP1 peptide or GLP1 peptide variant. The results are graphed as the percent change in food intake versus the plasma concentration of the HGE peptide (intact GLP1 peptide or GLP1 peptide variant) (FIGS. 6, 7, and 8). The results indicated that delivery of fusion proteins having SEQ ID NOs: 50, 33, 45, 46, and 44 resulted in GLP1R engagement at doses that were previously demonstrated to be efficacious for HSA-GDF15. Surprisingly, GLP1R engagement elicited a desired pharmacodynamic response similar to the Fc-GLP1 agonist dulaglutide despite having a GLP1 moiety exposure 10-30 times greater than dulaglutide. Had the GLP1 component of the fusion protein been as potent as dulaglutide in vivo, GLP1R engagement would have been much higher than desired, potentially causing predicted adverse events in humans such as nausea and emesis. Therefore, delivery of GLP1 peptides as fusions with HSA-GDF15 impacts in vivo potency at the GLP1R in a way that enables the desired balance of the two agonists.

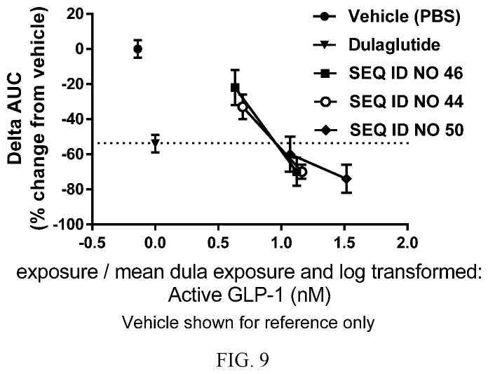

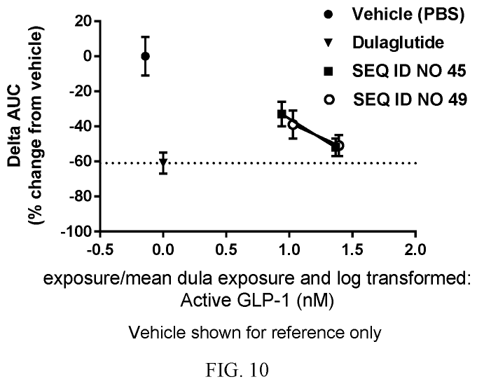

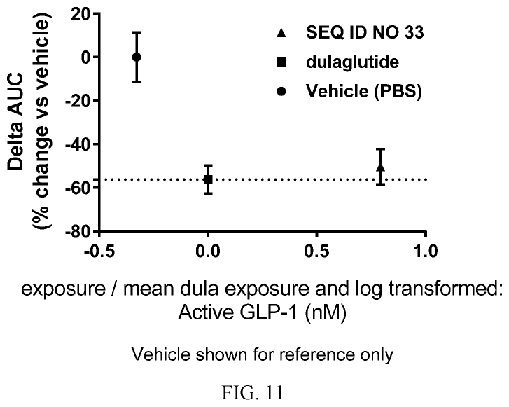

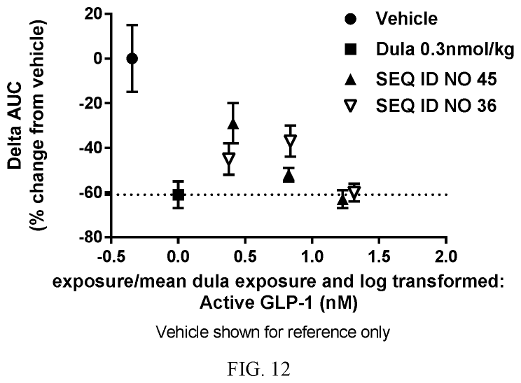

Assessment of GLP1R mediated glucose tolerance in fasted DIO mice: GLP1R agonism in the pancreas results in enhanced insulin secretion and increased glucose uptake and can be measured using an intraperitoneal glucose tolerance test in diet-induced obese mice. The objective of these experiments was to determine the extent of GLP1R engagement by the fusion proteins when they were administered at a dose predicted to be efficacious for HSA-GDF15 fusions in mice (FIGS. 1 and 2 and US20170327560A1). Male C57BL/6 mice were maintained on Research Diet D12492 from 6 weeks of age until the initiation of dosing at 20 weeks of age to induce obesity. Fed blood glucose and body weight measurements were used to randomize the mice into treatment groups. At 4:00 pm, mice were transferred to clean cages with access to water; food was withheld for the duration of the study. Control (PBS vehicle and dulaglutide) and test articles (SEQ ID NOs:33, 50, 49, 45, 46, 44, and 36) (1, 3, and 10 nmol/kg) were administered subcutaneously at the time of food removal. 17 hours after administration, baseline glucose was collected and 1 g of glucose was administered interperitoneally per kg of body weight. Blood glucose was measured at 10, 30, 60 and 120 minutes following the glucose bolus. Circulating plasma concentration of fusion molecules containing intact GLP1 peptide or GLP1peptide variants was determined immediately after the 120 minute time point by immuno-affinity capture-trypsin digestion-LC-MS/MS analysis and selective monitoring of tryptic peptides, HGE-1 (HGEGTFTSDVSSYLEEQAAK) (SEQ ID NO:77) or HGE-2 (HGEGTFTSDLSK) (SEQ ID NO:78) which are located near the N-terminus of the GLP1 peptide or GLP1 peptide variant. Glucose was graphed as a function of time and the area under the curve after normalizing to baseline glucose levels (Delta AUC) was calculated for each animal. The results are graphed as the percent change in Delta AUC (compared to vehicle treatment) versus the plasma concentration of the HGE peptide (intact GLP1 agonist) (FIGS. 9, 10, 11, and 12). The results indicated that delivery of fusion proteins having SEQ ID NOs:33, 50, 49, 45, 46, 44, and 36 resulted in GLP1R engagement at doses that were previously demonstrated to be efficacious for HSA-GDF15. Surprisingly, GLP1R engagement elicited a desired pharmacodynamics response similar to the Fc-GLP1 agonist dulaglutide despite having a GLP1 moiety exposure 10-30 times greater than dulaglutide. Had the GLP1 component of the fusion protein been as potent as dulaglutide in vivo, GLP1R engagement would have been much higher than desired, potentially causing predicted adverse events in humans such as nausea and emesis. Therefore, delivery of GLP1 peptides as fusions with HSA-GDF15 impacts in vivo potency at the GLP1R in a way that enables the desired balance of the two agonists.

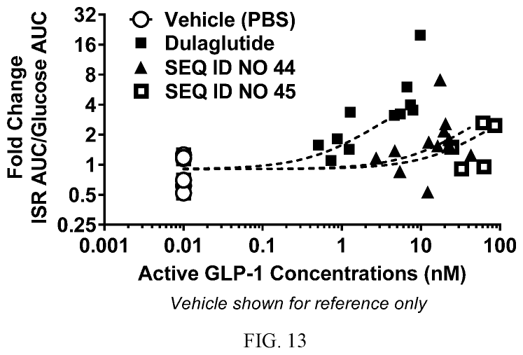

Assessment of GLP1R engagement in primates with a graded glucose infusion: Insulin secretion upon intravenous graded glucose infusion (GGI) in Non-Human Primates was used to assess GLP1R engagement of SEQ ID NOs:45 and 44. The objective of these experiments was to determine the extent of GLP1R engagement by the fusion proteins when the fusion proteins were administered at a dose predicted to be efficacious for HSA-GDF15 fusions in non-human primates (Mullican et al., 2017 and US20170327560A1). GGI procedures were conducted in sedated cynomolgus monkeys following a 16-hr overnight fast to compare the baseline and treatment responses with a 14-day recovery period between the two GGI procedures. On day 1, animals were dosed with vehicle immediately following food removal. On day 2, animals were anesthetized, and baseline was established with two blood samples collected 10 min apart. GGI1 was initiated at 0 minutes with a glucose infusion rate of 8 mg/kg/min, followed by infusions of 12, 16 and 24 mg/kg/min. Each infusion rate was administered for a period of 40 minutes. Blood samples were taken at 20-minute intervals for measurement of glucose, insulin, and C-peptide, results were graphed as a function of time and area under the curve (AUC) was determined for each analyte measured in each animal. Animals were then randomized into test article treatment groups based on their baseline insulin secretion rate (ISR) from GGI1. On day 15 animals were dosed with the test article at varying dose concentrations (0.1 to 1.1 mg/kg of fusion proteins and 0.016 to 0.1 dulaglutide) prior to fasting overnight. The second GGI (GGI2) was performed on day 16 as described for day 2. Compound exposure was assessed on plasma samples collected at timepoint 0 prior to glucose infusion by immunoassay specifically designed to monitor test articles with intact GLP1 peptide or peptide variants. Data in FIG. 13 is reported as the treatment induced fold change in ISR AUC normalized to glucose AUC compared to baseline versus the plasma concentration of test article with intact GLP1. Dulaglutide was used as a Fc-GLP1 agonist reference. The results indicated that delivery of fusion proteins having SEQ ID NOs:45 and 44 resulted in GLP1R engagement at doses that were previously demonstrated to be efficacious for HSA-GDF15. Surprisingly, GLP1R engagement elicited a desired pharmacodynamic response similar to Fc-GLP1 agonist dulaglutide despite having a GLP1 moiety exposure 10-30 times greater than dulaglutide. Had the GLP1 component of the fusion protein been as potent as dulaglutide in vivo, GLP1R engagement would have been much higher than desired, potentially causing predicted adverse events in humans such as nausea and emesis. Therefore, delivery of GLP1 peptides as fusions with HSA-GDF15 impacts in vivo potency at the GLP1R in a way that enables the desired balance of the two agonists.

The results shown in FIGS. 6-13 demonstrate that GLP1 in vivo activity or potency in preclinical species is reduced in the homodimeric GLP1-GDF15 dual agonists compared with the Fc fused GLP1 peptide of dulaglutide. This enables balance of both agonists (GLP1 peptide or GLP1 peptide variant and GDF15 protein or GDF15 protein variant) on one molecule; therefore, allowing delivery of a GLP1 peptide or GLP1 peptide variant at an efficacious concentration that also achieves GDF15 protein or GDF15 protein variant exposure within the predicted therapeutic range.

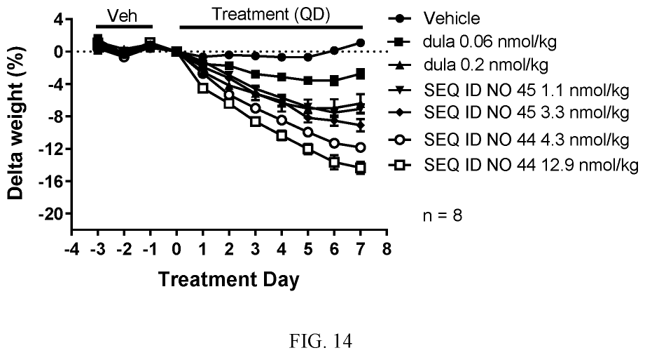

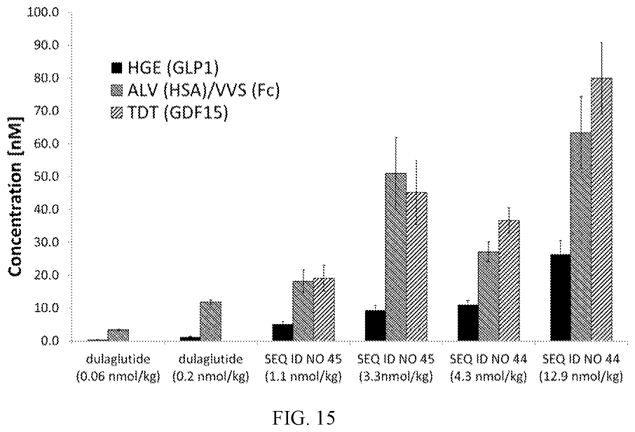

Assessment of weight loss efficacy of GLP1-GDF15 fusion proteins after repeat dosing in mice: The ability of GLP1-GDF15 fusion proteins to cause weight loss was tested in diet induced obese mice. Mice were subcutaneously administered varying concentrations of test articles (SEQ ID NOS:45 and 44), dulaglutide or a vehicle control daily for seven days. Body weight was monitored throughout the study and FIG. 14 shows the percent change in body weight over time relative to Day 0 (just prior to the first dose). Circulating concentration of test articles was determined after seven days of treatment by immuno-affinity capture-trypsin digestion-LC-MS/MS analysis. Selected tryptic peptides were monitored, namely, ALV (ALVLIAFAQYLQQSPFEDHVK) (SEQ ID NO:79), HGE-1 (HGEGTFTSDVSSYLEEQAAK) (SEQ ID NO:77), and HGE-2 (HGEGTFTSDLSK) (SEQ ID NO:78), and TDT (TDTGVSLQTYDDLLAK) (SEQ ID NO:80), which are located near the N-terminus of the HSA protein, the N-terminus of the GLP1 peptide or GLP1 peptide variant, and the C-terminus of the GDF15 protein or GDF15 protein variant, respectively. The VVS peptide (VVSVLTVLHQDWLNGK) (SEQ ID NO:82) is located in the Fc portion of dulaglutide. Monitoring these surrogate peptides enabled pharmacokinetic assessment of each region (GLP1, HSA, GDF15) of the GLP1-GDF15 fusion proteins. The plasma drug concentrations are shown in FIG. 15. The results indicated that repeat administration of SEQ ID NOs:45 and 44 leads to weight loss in diet induced obese mice in a concentration dependent manner.

### Assessment of Food Intake Effects of GLP1-GDF15 Fusion Proteins in Nonhuman Primates

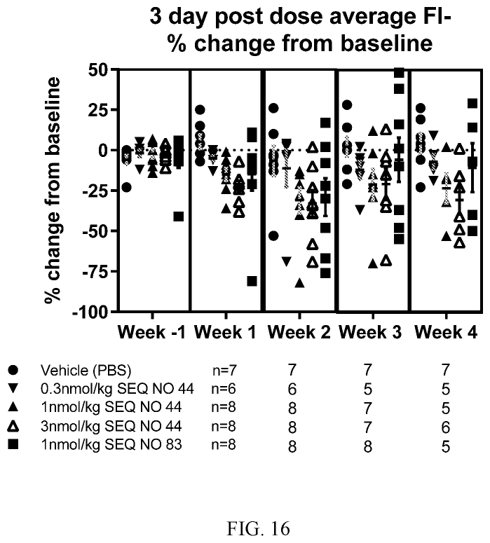

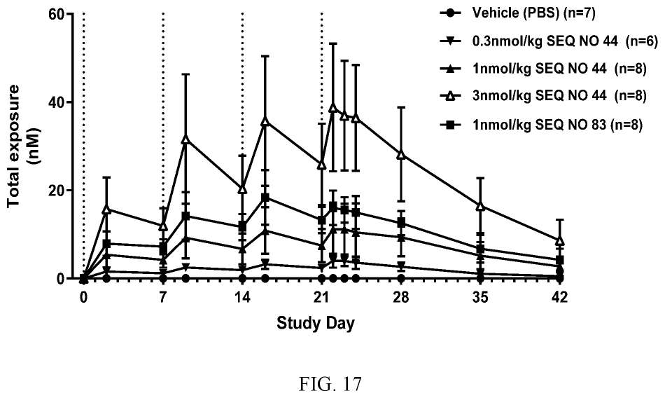

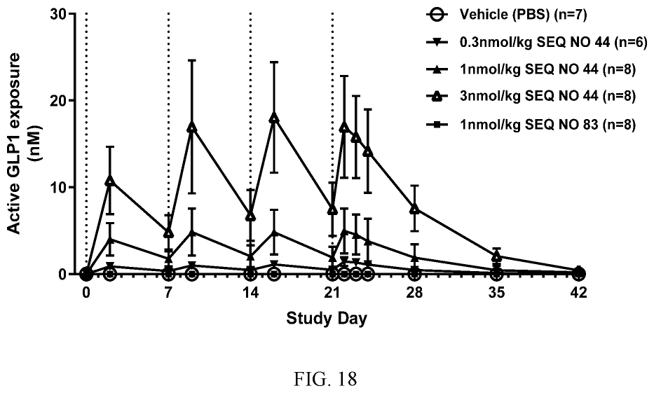

The ability of GLP1-GDF15 fusion proteins to decrease food intake was tested in spontaneously obese cynomolgus monkeys. Subjects were subcutaneously administered varying concentrations of test articles with and without an active GLP1 moiety (SEQ ID NOS: 44 and 83, respectively; SEQ ID NO:83, which comprises a HSA peptide fused to an AP10 linker fused to a GDF15 peptide, was previously disclosed in U.S. Pat. No. 10,336,812) or vehicle QW for a period of 4 weeks. Food intake was monitored prior to dosing and throughout the study. The 3-day percent change in food intake at baseline and after each administration of test article is shown in FIG. 16. The concentration of SEQ ID NOs: 44 and 83 in cynomolgus monkey plasma during the study was determined using two separate immunoassay methods to detect the “total” molecule (HSA detection) and the “active” GLP1 moiety (N-terminal GLP1 peptide detection). The plasma drug concentrations are shown in FIGS. 17 and 18. The results indicated that administration of SEQ ID NOs: 44 and 83 reduces food intake in spontaneously obese nonhuman primates.

### Assessment of Food Intake and Body Weight Effects of GLP1-GDF15 Fusion Proteins After a 21-Day Dose Escalation in Nonhuman Primates

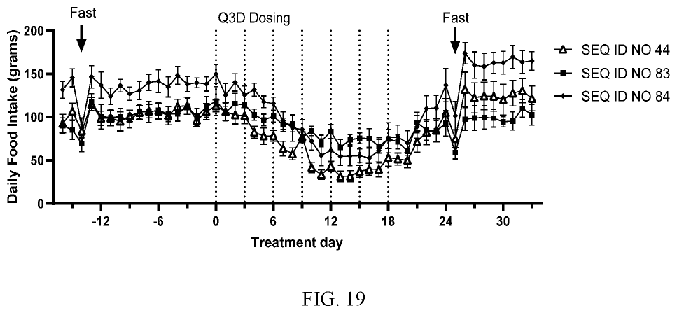

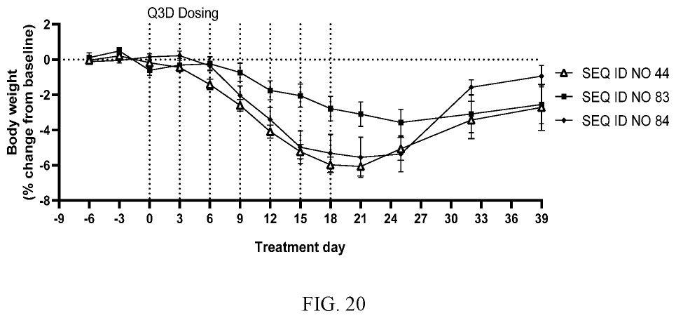

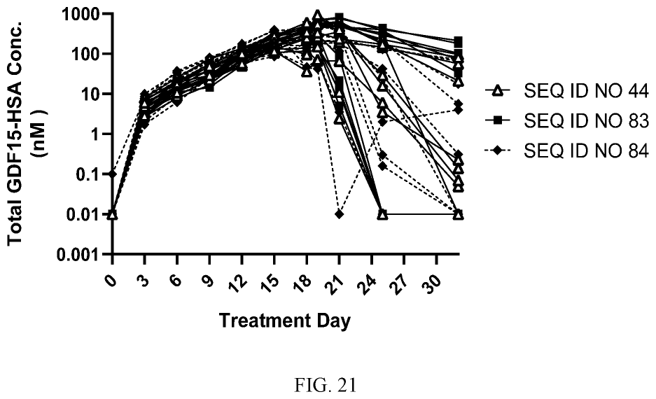

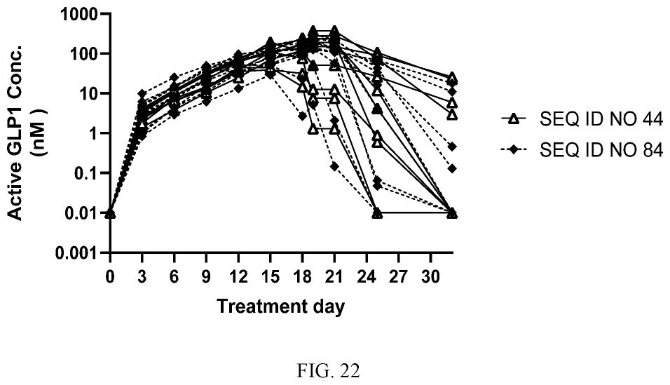

The ability of GLP1-GDF15 fusion proteins to decrease food intake and cause weight loss was tested in spontaneously obese cynomolgus monkeys. Subjects were subcutaneously administered test articles containing both active GDF15 and GLP1, only active GDF15, or only active GLP1 moieties (SEQ ID NOS: 44, 83, 84, respectively; SEQ ID NO:84 comprises a GLP1 peptide fused to an AP5 linker peptide fused to a HSA peptide fused to an AP10 linker peptide fused to a GDF15 peptide with an I89R mutation; the GDF15 peptide with an I89R mutation was previously disclosed in U.S. Pat. No. 10,336,812) every three days for a period of 21 days. The initial dose given at Day 0 was 0.7 nmol/kg and was escalated upon each administration up to 20 nmol/kg. Daily food intake prior to dosing, during the 21-day treatment period and during test article washout is shown in FIG. 19. Body weight was monitored throughout the study and FIG. 20 shows the percent change in body weight over time relative to baseline. The concentration of test articles in cynomolgus monkey plasma during the study was determined using two separate immunoassay methods to detect the “total” molecule (HSA detection; SEQ ID NOS: 44, 83, 84) and the “active” GLP1 moiety (N-terminal GLP1 peptide detection; for SEQ ID NOS: 44 and 84 only). The plasma drug concentrations are shown in FIGS. 21 and 22. The results indicated that repeat administration of SEQ ID NOs: 44, 83, 84 leads to a reduction in food intake and subsequent weight loss in spontaneously obese nonhuman primates.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

It is claimed:

1. A glucagon-like peptide-1 (GLP1)/growth differentiation factor 15 (GDF15) fusion protein, wherein the GLP1-GDF15 fusion protein comprises a GLP1 peptide, a first linker peptide, a serum albumin protein, a second linker peptide, and a GDF15 protein.

2. The GLP1-GDF15 fusion protein of claim 1, wherein GLP1 peptide comprises an amino acid sequence selected from the group consisting of SEQ ID NOs:1-4.

3. The GLP1-GDF15 fusion protein of claim 1, wherein the first linker peptide comprises an amino acid sequence selected from the group consisting of SEQ ID NOs:5-25.

4. The GLP1-GDF15 fusion protein of claim 1, wherein the serum albumin protein comprises an amino acid sequence selected from SEQ ID NO:26 or SEQ ID NO:27.

5. The GLP1-GDF15 fusion protein of claim 1, wherein the second linker peptide comprises an amino acid sequence selected from the group consisting of SEQ ID NOs:28-30.

6. The GLP1-GDF15 fusion protein of claim 1, wherein the GDF15 protein comprises an amino acid sequence selected from SEQ ID NO:31 or SEQ ID NO:32.

7. The GLP1-GDF15 fusion protein of claim 1, wherein the GLP1-GDF15 fusion protein comprises an amino acid sequence selected from the group consisting of SEQ ID NOs:73, 74 and 84.

8. An isolated nucleic acid encoding the GLP1-GDF15 fusion protein of claim 1.

9. A vector comprising the isolated nucleic acid of claim 8.

10. A host cell comprising the isolated nucleic acid of claim 8.

11. A pharmaceutical composition comprising the GLP1-GDF15 fusion protein of claim 1 and a pharmaceutically acceptable carrier.

12. A method for treating or preventing a disease or disorder in a subject in need thereof, wherein said disease or disorder is selected from the group consisting of obesity, type I or type II diabetes, metabolic syndrome, insulin resistance, impaired glucose tolerance, hyperglycemia, hyperinsulinemia, hypertriglyceridemia, hypoglycemia due to congenital hyperinsulinism (CHI), dyslipidemia, atherosclerosis, diabetic nephropathy, and other cardiovascular risk factors such as hypertension and cardiovascular risk factors related to unmanaged cholesterol and/or lipid levels, osteoporosis, inflammation, non-alcoholic fatty liver disease (NAFLD), non-alcoholic steatohepatitis (NASH), renal disease, and eczema, the method comprising administering to the subject in need thereof an effective amount of the pharmaceutical composition of claim 11.

13. A method of reducing food intake in a subject in need thereof, the method comprising administering to the subject in need thereof an effective amount of the pharmaceutical composition of claim 11.

14. A method of modulating GLP1 receptor activity in a subject in need thereof, the method comprising administering to the subject in need thereof an effective amount of the pharmaceutical composition of claim 11.

15. A method of modulating GDF15 receptor (GFRAL) activity in a subject in need thereof, the method comprising administering to the subject in need thereof an effective amount of the pharmaceutical composition of claim 11.

16. The method of claim 12, wherein the pharmaceutical composition is administered via an injection.

17. A kit comprising the GLP1-GDF15 fusion protein of claim 1.

18. The kit of claim 17, wherein the kit further comprises a device for injection.

19. A method of producing a pharmaceutical composition comprising the GLP1-GDF15 fusion protein of claim 1, comprising combining the GLP1-GDF15 fusion protein with a pharmaceutically acceptable carrier to obtain the pharmaceutical composition.

20. A method of producing the GLP1-GDF15 fusion protein of claim 1, comprising culturing a cell comprising a nucleic acid encoding the GLP1-GDF15 fusion protein under conditions to produce the GLP1-GDF15 fusion protein, and recovering the GLP1-GDF15 fusion protein from the cell or culture.
